(19) United States
(12) Patent Application Publication
(10) Pub. No.: US 2024/0062912 A1
(43) Pub. Date: Feb. 22, 2024

(54) METHODS FOR DETERMINING LIVER FUNCTION COMPRISING A MULTI-COMPARTMENTAL MODEL

(71) Applicant: HepQuant, LLC, Denver, CO (US)

(72) Inventors: Michael P. McRae, Houston, TX (US); Steve Mark Helmke, Denver, CO (US); Gregory Thomas Everson, Englewood, CO (US)

(21) Appl. No.: 18/446,164

(22) Filed: Aug. 8, 2023

Related U.S. Application Data
(60) Provisional application No. 63/421,876, filed on Nov. 2, 2022.
(60) Provisional application No. 63/396,131, filed on Aug. 8, 2022.

(51) Int. Cl. G16H 50/30; G16C 20/30; G01N 33/58; G16H 50/50

(52) U.S. Cl. CPC G16H 50/30; G16C 20/30; G01N 33/58; G16H 50/50

(57) ABSTRACT

Methods are provided for determining liver function based on a Compartmental Model (CM) method for analysis of cholate SHUNT test data which closely approximates and elucidates the underlying physiology of hepatic uptake of cholate from systemic and portal circulations simultaneously.

### CROSS-REFERENCES TO RELATED APPLICATIONS

This application claims the benefit of priority to U.S. Provisional Application Ser. No. 63/396,131, filed Aug. 8, 2022, and U.S. Provisional Application Ser. No. 63/421,876, filed Nov. 2, 2022, each of which is incorporated herein by reference in its entirety.

### BACKGROUND OF THE INVENTION

Chronic liver disease (CLD) affects millions worldwide. Estimates suggest that as many as 50 million Americans have CLD, with over 15 million with fibrosis, and over 4 million with cirrhosis. The common etiologies of CLD include alcoholic liver disease, chronic viral hepatitis B or C, autoimmune hepatitis, primary biliary cholangitis, primary sclerosing cholangitis, and genetic conditions. But, as a result of the obesity pandemic, the most common emerging and expanding etiology for CLD is nonalcoholic fatty liver disease (NAFLD) or nonalcoholic steatohepatitis (NASH). Costs of CLD could exceed $30 billion by 2025. The HepQuant cholate SHUNT test quantifies liver health (DSI, disease severity index) to aid providers, patients, and payers in the management of CLD patients.

The HepQuant cholate SHUNT test quantifies liver function from portal and systemic clearances simultaneously using stable nonradioactive isotopes of carbon-13-labeled cholate (13C-CA) intravenously and deuterium-labeled cholate (d4-CA) orally. The test is minimally invasive, well tolerated, and only requires the sampling of peripheral venous blood. Systemic concentrations of cholate are measured in serum by advanced liquid chromatography/mass spectrometry (LC/MS) or LC/MS-MS methods using validated single quadrupole and triple quadrupole MS. HepQuant provides an innovative disease severity index (DSI) score, a single score of liver health ranging from 0 (healthy) to 50 (severely impaired). This DSI has demonstrated reliability in reproducibility studies [Burton J R, Helmke S, Lauriski S, Kittelson J, Everson G T. The within-individual reproducibility of the disease severity index from the HepQuant SHUNT test of liver function and physiology. Transl Res. 2021; 233:5-15] and has detected early stages of disease, quantified disease severity, monitored disease progression, and measured treatment effects [Helmke S, Colmenero J, Everson G T. Noninvasive assessment of liver function. Curr Opin Gastroenterol. 2015; 31]. The test uniquely enables pharmaceutical companies to measure efficacy of their therapeutics by measuring improvements in liver function rather than waiting to clinically observe progressive liver failure. A pivotal study has been recently completed linking HepQuant test results to the likelihood of large esophageal varices, a precursor to variceal hemorrhage and the most lethal complication of CLD [ClinicalTrials.gov. The SHUNT-V Study for Varices NCT03583996].

The HepQuant cholate SHUNT test of liver function and physiology can be useful for monitoring treatment effects and predicting risk for clinical outcome. The HepQuant cholate SHUNT test quantifies hepatic functional impairment from the simultaneous clearance of cholate from the systemic and portal circulations for the purpose of monitoring treatment effects or for predicting risk for clinical outcome. Test parameters, derived from a noncompartmental minimal model (MM), are reproducible and reliable (Translational Research 2021).

Currently, the shunt fraction (SHUNT) quantified by the HepQuant SHUNT test calculates the combined contributions of both hepatic extraction efficiency and anatomic shunts in diseased subjects; however, the test incompletely differentiates these two contributions. Defining the relative contributions of these two mechanisms could add novel clinical utility to the HepQuant cholate SHUNT test by identifying which elements of liver function and physiology may be affected by treatments or interventions. In addition, incorporating physiological information into the pharmacokinetic modeling could reduce intra-individual/biological variability associated with the test's response variables.

A new method comprising compartmental analysis of the HepQuant SHUNT test is desirable because it could uncover underlying mechanisms of hepatic functional impairment associated with disease progression or resolution.

### SUMMARY OF THE INVENTION

The present disclosure provides a Compartmental Model (CM) method for analysis of cholate SHUNT test data which more closely approximates and elucidates the underlying physiology of hepatic uptake of cholate from systemic and portal circulations simultaneously. The CM results are compared to the previously validated MM in terms of test reproducibility.

The compartmental model correlates well to a previously validated noncompartmental MM model with R² of 0.96. Acceptable reproducibility (intraclass correlation coefficient>0.7) was observed for 6/6 disease indices for the compartmental model versus ⅚ for the noncompartmental model. The compartmental model reduced individual/biological variability and quantified both anatomic shunt and hepatic extraction, potentially improving the diagnostic performance of the HepQuant cholate SHUNT test.

A method is provided for assessing liver function in a subject having or suspected of having or contracting a liver disease, comprising obtaining blood or serum sample concentration data of an orally administered first distinguishable cholate compound in samples collected from a subject at a multiplicity of time points after oral administration; obtaining blood or serum sample concentration data of an intravenously administered second distinguishable cholate compound in the samples collected from the subject at the multiplicity of time points after simultaneous intravenous co-administration; fitting the first and second distinguishable cholate concentration data to a physiological-based compartmental model of dual cholate clearance to obtain fitted data, the compartmental model comprising body mass index (BMI), body weight (BW), and hematocrit (Hct) input values in the subject; and calculating one or more indices of hepatic disease in the subject using the fitted data, wherein the one or more indices is associated with liver function in the subject. The method may further comprise providing the one or more indices of hepatic disease to a medical professional for the purpose of developing a treatment plan in the subject.

The one or more indices of hepatic disease may be employed for a purpose selected from the group consisting of determining a need for treatment, predicting response to treatment, monitoring the effectiveness of a treatment, and predicting risk of clinical outcome in the subject.

The physiological-based compartmental model may comprise algorithms for (i) estimating compartment volumes of a plurality of compartments in the subject; (ii) estimating flow parameters between the plurality of compartments in the subject; and (iii) estimating hepatic extraction of the intravenously administered distinguishable cholate in the subject. The physiological-based compartmental model may comprise an algorithm for estimating cholate binding, and dose administration in the subject.

The physiological-based compartmental model may comprise a plurality of compartments in the subject. The plurality of compartments may include two or more, three or more, one to ten, two to eight, three to seven, or three physiological compartments in the subject. The plurality of compartments may comprise systemic, portal, and liver compartments in the subject.

The estimating compartment volumes of a plurality of compartments in the subject may comprise estimating the systemic compartment volume (V_{S}), the portal compartment volume (V_{P}), and the liver compartment volume (V_{L}) in the subject.

The estimating of the systemic compartment volume (V_{S}) may comprise

V_{S}=TBV·fₚₗₐₛₘₐ, wherein
TBV is total blood volume in the subject:

$$TBV = \frac{0.07 \cdot BW}{\sqrt{BMI / 22}} ,$$

wherein
BMI is body mass index (kg/m²) in the subject; BW is body weight (kg) in the subject; and fₚₗₐₛₘₐ is the plasma fraction in the subject: fₚₗₐₛₘₐ=(1−Hct), wherein Hct is the hematocrit in the subject.

The estimating of the portal compartment volume (V_{P}) may comprise

V_{P}=0.25·V_{S}.

The estimating of the liver compartment volume (V_{L}) may comprise

V_{L}=(0.275·22.46·BW·d_{L}·fₚₗₐₛₘₐ)/1000,
wherein d_{L} is Liver tissue density of 1.06 g·mL⁻¹.

The estimating the flow parameters between the plurality of compartments in the subject may comprise describing the intravenously and orally administered distinguishable cholate concentrations in each of the systemic (C_{S}), portal (C_{P}), and liver (C_{L}) compartments.

The describing the intravenously and orally administered distinguishable cholate concentrations in each of the systemic (C_{S}), portal (C_{P}), and liver (C_{L}) compartments may comprise calculating: initial estimate of total hepatic inflow (Q_{L, init}), total hepatic inflow (Q_{L}), splanchnic arterial circulation (q_{SP}), hepatic portal venous inflow to the liver (q_{PL}), total hepatic venous return flow to systemic circulation (q_{LS}), hepatic arterial inflow to the liver (q_{SL}), and anatomic shunt flow (q_{PS}) rates in the subject.

The calculating of the initial estimate of total hepatic inflow to the liver (Q_{L, init}) in the subject may comprise

Q_{L,init}=1·w_{L}·fₚₗₐₛₘₐ, (L·min⁻¹·kg⁻¹), wherein
w_{L} is total liver weight (blood weight plus parenchymal weight): w_{L}=22.46·BW·d_{L}.

The calculating total hepatic inflow to the liver (Q_{L}) in the subject may comprise

Q_{L}=q_{SL}+q_{PL}, (L·min^{31 1})
wherein
q_{SL} is rate of hepatic arterial inflow to the liver,

q_{SL}=0.25·Q_{L, init} (L·min⁻¹); and
q_{PL} is rate of portal venous inflow to the liver,

q_{PL}=q_{SP}−q_{PS} (L·min⁻¹), wherein
q_{SP} is splanchnic arterial blood flow rate to abdominal intestinal organs,

q_{SP}=0.75·Q_{L, init} (L·min⁻¹), and
q_{PS} is anatomic shunt flow rate, (L·min⁻¹) which bypasses the liver.

The calculating total hepatic venous return flow rate to systemic circulation (q_{LS}) in the subject may comprise: q_{LS}=q_{PL}+q_{SL}.

The estimating the hepatic extraction in the subject may comprise calculating a fast phase extraction ratio (ER_{fast}), fast phase clearance of intravenously administered distinguishable cholate (Cl_{IV},), and slow phase of hepatic clearance (Cl_{H}) in the subject.

The fast phase extraction ratio (ER_{fast}) may be calculated as the ratio of total IV clearance (Cl_{IV}) (0 to 180 min) to first phase clearance (Cl_{FP}) (0-20 min)

$${ER}_{fast} = \frac{{Cl}_{IV}}{{Cl}_{FP}} = \frac{{C}_{0}}{{k}_{fast} \cdot {AUC}_{IV}} ,$$

wherein
AUC_{IV} is the area under the IV curve; k_{fast} is estimated from the slope of log concentration-time IV curves between 5- and 20-minute timepoints,

$${k}_{fast} = - \frac{ln ( C [ 20 ] ) - ln ( C [ 5 ] )}{t ( 20 ) - t ( 5 )} ( {min}^{- 1} ) ;$$

and
and C₀ is the extrapolated initial intravenously administered distinguishable cholate concentration

$${C}_{0} = - \frac{C ( 5 )}{{e}^{- {k}_{fast} \cdot t ( 5 )}} ( μ mol \cdot {L}^{- 1} ) ,$$

wherein
C(5) is the concentration of intravenously administered distinguishable cholate in the sample collected at 5 minutes after administration.

C[20] may be the concentration of intravenously administered distinguishable cholate in the sample collected at 20 minutes after administration. C[5] may be the concentration of intravenously administered distinguishable cholate in the sample collected at 5 minutes after administration.

The fast phase clearance of intravenously administered distinguishable cholate (Cl_{IV}) may be calculated as

Cl_{IV}=Q_{L}·ER_{fast} (L·min^{31 1}).

The slow phase of hepatic clearance (Cl_{H}) in the subject is the hepatic clearance of orally administered distinguishable cholate and slow phase of intravenously administered distinguishable cholate clearance in the subject, optionally calculated as

Cl_{H}=Q_{L}·ER_{slow} (L·min^{31 1}).

The estimating cholate binding in the subject may comprise (1) modeling albumin-bound noncellular intravascular distinguishable cholate, (2) modeling extravascular distinguishable cholate distributed to cells/tissues, and (3) modeling free/unbound distinguishable cholate in the subject.

The modeling of albumin-bound noncellular intravascular distinguishable cholate in the subject may comprise

estimating an initial albumin-bound noncellular intravascular fraction of total dose estimate (f_{A, init}) comprising calculating the ratio of systemic compartment volume (Vs) to the volume of distribution (Vd)

f_{A, init}=Vs/Vd,

wherein V_{d}=D_{IV}/C₀ (L), D_{IV} is intravenous dose of distinguishable cholate, and C₀ and Vs are as defined above.

The actual intravascular fraction (f_{A}) may be determined comprising parameter estimation comprising weighted nonlinear least-squares regression at each distinguishable cholate concentration.

The weighted nonlinear least-squares regression at each distinguishable cholate concentration may be reduced to reflect higher uncertainty during rapid distribution phase of IV clearance, and at low concentrations of oral clearance. The 5 min IV distinguishable cholate weight may be weighted as is 0.33, and the 5 min and 90 min oral distinguishable cholate may be weighted as 0.67.

The one or more indices of hepatic disease may be selected from the group consisting of portal hepatic filtration rate (HFRp), systemic hepatic filtration rate (HFRs), cholate SHUNT, STAT, liver disease severity index (DSI), and hepatic reserve (HR) in the subject.

The portal hepatic filtration rate (HFRp) is a clearance adjusted for body weight (BW) of the subject, which may be calculated as

$${HFR}_{P} = \frac{{D}_{PO}}{{AUC}_{PO} \cdot BW} ,$$

wherein
D_{PO} is oral dose of the distinguishable cholate; and
AUC_{PO} is area under the oral distinguishable cholate blood or serum sample concentration curve over the multiplicity of time points after administration.

The systemic hepatic filtration rate (HFRs) is a clearance adjusted for body weight (BW) of the subject, which may be calculated as

$${HFR}_{S} = \frac{{D}_{IV}}{{AUC}_{IV} \cdot BW} ,$$

wherein
D_{IV} is intravenous dose of the distinguishable cholate;
AUC_{IV} is area under the intravenous distinguishable cholate blood or serum sample concentration curve over the multiplicity of time points after administration.

The cholate SHUNT (F) is a comparison of bioavailability of orally administered distinguishable cholate with bioavailability of IV-administered distinguishable calculated by the ratio of areas under the oral and IV distinguishable cholate concentration curves (AUC_{PO} and AUC_{IV}, respectively) and normalized by the IV dose (D_{IV}) and oral dose (D_{PO}), optionally wherein the SHUNT is calculated comprising

$$F = \frac{{AUC}_{PO} \cdot {D}_{IV}}{{AUC}_{IV} \cdot {D}_{PO}} .$$

The STAT is a single point blood or serum oral distinguishable cholate concentration, which may be used to estimate a liver disease severity index (DSI) score.

The DSI is a score indicative of overall liver function comprising both portal and systemic HFR values, optionally wherein

$$DSI = A \cdot \sqrt{{( ln [ \frac{{HFR}_{P , max}}{{HFR}_{P}} ] )}^{2} + {( ln [ \frac{{HFR}_{S , max}}{{HFR}_{S}} ] )}^{2}} ,$$

wherein HFR_{P,max} and HFR_{S,max} are the upper limits of clearance of healthy controls, and
A is a factor to scale DSI score from 0 to 50.

The hepatic reserve (HR) is a numerical index representing overall hepatic health, and HR values may be a value between 0 and 100. The HR may be calculated comprising

$$HR = 100 - A \cdot \sqrt{{( ln [ \frac{{HFR}_{P , lean}}{{HFR}_{P}} ] )}^{2} + {( ln [ \frac{{HFR}_{S , lean}}{{HFR}_{S}} ] )}^{2}} ,$$

wherein
HFR_{P} and HFR_{S} are indexed to lean controls minus one standard deviation (HFR_{P,lean} and HFR_{S,lean}); and A is a constant integer from 100 to 0.

The first distinguishable cholate compound and the second distinguishable cholate compound may be different. In some examples, the first distinguishable cholate compound may be a first stable isotope labeled cholate and the second distinguishable cholate compound may be a second stable isotope labeled cholate. In some examples, the first and second stable isotope labeled cholates may be selected from 2,2,4,4-d4 cholate and 24-¹³C-cholate.

The physiological-based compartmental model of dual cholate clearance methods according to the disclosure may further comprise receiving a plurality of blood or serum samples collected from the subject having or suspected of having or contracting a chronic liver disease, following oral administration of a dose of the first distinguishable cholate compound (DPO) to the subject and simultaneous intravenous co-administration of a dose of a second distinguishable cholate compound (DIV) to the subject, wherein the samples have been collected over intervals spanning a period of time after administration; and quantifying the concentration of the first and the second distinguishable cholate compounds in each sample.

The fitting of the first and second distinguishable cholate concentration data to a physiological-based compartmental model may comprise generating an individualized oral clearance curve from the concentration of the first distinguishable cholate compound in each sample comprising using a computer algorithm curve fitting to the compartmental model and computing the area under the individualized oral clearance curve (AUC_{PO}).

The fitting of the first and second distinguishable cholate concentration data to a physiological-based compartmental model may comprise generating an individualized intravenous clearance curve from the concentration of the second distinguishable cholate compound in each sample by use of a computer algorithm curve fitting to the compartmental model and computing the area under the individualized intravenous clearance curve (AUC_{IV}).

In some examples, the blood or serum samples have been collected from the subject over intervals comprising from two to seven time points, three to six time points, four to five time points, or one, two, three, four, five, six, or seven, or more time points after administration. The samples have been collected from the patient at about 0, about 5, about 20, about 45, about 60, and about 90 minutes after administration. In some examples, the blood or serum samples have been collected from the subject over intervals for no more than about 180 minutes, about 120 minutes, or about 90 minutes after administration of the first and second distinguishable cholates.

In some examples, the subject is a human subject.

The liver disease may be a chronic liver disease. The chronic liver disease may be selected from the group consisting of chronic hepatitis C (CHC), chronic hepatitis B, alcoholic liver disease, Alcoholic SteatoHepatitis (ASH), and Non-Alcoholic Fatty Liver Disease (NAFLD), steatosis, Non-Alcoholic SteatoHepatitis (NASH), autoimmune liver disease, cryptogenic cirrhosis, hemochromatosis, Wilson's disease, alpha-1-antitrypsin deficiency, liver cancer, liver failure, cirrhosis, primary sclerosing cholangitis (PSC), and other cholestatic liver diseases.

In some examples, the clinical outcome is selected from the group consisting of Child-Turcotte-Pugh (CTP) progression, Model for End-stage Liver Disease (MELD) progression, variceal hemorrhage, ascites, splenomegaly, varices, portal hypertension (PHTN), hepatic encephalopathy, hepatocellular carcinoma (HCC), decompensation, or liver-related death.

The treatment may be selected from any appropriate treatment known in the art. The treatment may be selected from the group consisting of antiviral treatments, antifibrotic treatments, antibiotics, immunosuppressive treatments, anti-cancer treatments, ursodeoxycholic acid, farnesoid X receptor ligands, insulin sensitizing agents, interventional treatment, liver transplant, lifestyle changes, dietary restrictions, low glycemic index diet, antioxidants, vitamin supplements, transjugular intrahepatic portosystemic shunt (TIPS), catheter-directed thrombolysis, balloon dilation and stent placement, balloon-dilation and drainage, weight loss, exercise, and avoidance of alcohol.

In some examples, the determining a need for treatment, predicting response to treatment, monitoring the effectiveness of a treatment, or predicting risk of clinical outcome in the subject may comprise determining the one or more indices of hepatic disease in the subject; and comparing the one or more indices of hepatic disease to one or more cutoff value(s), wherein a change in the one or more indices of hepatic disease compared to cutoff value(s) is indicative of the need for treatment, response to treatment, or effectiveness of the treatment, respectively, in the subject. In some examples, the cutoff value(s) may be derived from one or more normal healthy controls, a group of known patients, or within the subject over time.

The group of known patients may be suffering from a disease or condition, optionally selected from patients having a fibrosis stage; portal hypertension; Childs-Turcotte-Pugh (CTP) score A; CTP score B, CTP score C; Model for End-stage Liver Disease (MELD) progression score, primary sclerosing cholangitis (PSC) not listed for transplant; PSC listed for liver transplant; PSC listed for liver transplant without varices; PSC listed for liver transplant with varices; ascites; stomal bleeding; splemomegaly; varices; large varices, variceal hemorrhage; hepatic encephalopathy, decompensation; or liver disease-related death.

The fibrosis stage may be determined by a method comprising liver biopsy or elastography. The liver biopsy may determine an Ishak fibrosis score selected from the group consisting of F2 (mild portal fibrosis), F3, F4 (moderate bridging fibrosis), F5 (nodular formation and incomplete cirrhosis), and F6 (cirrhosis).

The anatomic shunt flow rate (q_{PS}) may be used to differentiate healthy controls from subjects with large varices.

The rate of portal venous inflow to the liver (q_{PL}) may be used to differentiates healthy controls, subjects with no varices, subjects with small varices, and subjects with large varices.

### BRIEF DESCRIPTION OF THE DRAWINGS

FIG. 1 shows a diagram illustrating the compartmental model of dual cholate clearance according to the present disclosure describing the transfer of cholate between systemic, portal, and liver compartments. D_{IV}=intravenous dose of 13C cholate; D_{PO}=oral dose of d4-CA; q_{SP}=splanchnic arterial flow rate; qₚₛ=anatomic shunt flow rate; q_{PL}=portal venous flow rate; q_{LS}=hepatic return to systemic circulation flow rate; q_{SL}=hepatic arterial flow rate; Cl_{H}=hepatic clearance.

FIG. 2A shows a graph of distinguishable cholate concentration in a control subject over 180 minutes after simultaneous administration of IV and PO distinguishable cholates as compared in a Compartmental Model vs. prior art Minimal Model analyses. CM (solid lines) and MM (dotted lines) fits to 13C-CA (red) and d4-CA (green) concentration measurements from a control subject

FIG. 2B shows a graph of distinguishable cholate concentration in a NASH subject over 180 minutes after simultaneous administration of IV and PO distinguishable cholates as compared in a Compartmental Model vs. prior art Minimal Model analyses. CM (solid lines) and MM (dotted lines) fits to 13C-CA (red) and d4-CA (green) concentration measurements from the NASH subject

FIG. 3 shows four correlation plots comparing CM and MM. Correlation plots compare DSI (panel A) and HR (panel C) values from each model with Deming regression (solid black line). Bland-Altman plots show agreement between the two methods for calculating DSI (panel B) and HR (panel D) with upper and lower limits of agreement (dotted lines).

FIG. 4 shows parameter scans of anatomic shunt (left panel) and extraction ratio (right panel). Simulated concentrations of 13C-CA (dotted lines) and d4-CA (solid lines) were generated while varying the anatomic shunt flow rate (qps) at five levels between no shunting and full shunting (0, 25, 50, 75, and 100% of splanchnic flow rate) and normal ER values. Similarly, the effect of varying ER while all other parameters remained constant was investigated at five levels (0.8, 0.6, 0.4, 0.2, and 0.1). Here, both ER_{fast} and ER_{slow} were considered equal.

FIG. 5 shows a cartoon of compartmental modeling of the cholate SHUNT test in health (left panel) and in liver disease (right panel) showing the relationship between systemic, portal, and liver compartments. In health, there are no anatomic shunts and measured spillover of d4-CA to systemic compartment (SHUNT fraction) defines the liver's normal extraction efficiency. In liver disease, anatomic shunts between portal and systemic circulation bypass the liver and extraction efficiency decreases. One objective of the compartmental model is to define the relative contributions of anatomic shunt and hepatic extraction.

FIG. 6A shows a graph of Compartmental model fit of the data with median adjusted R² of 0.99 for 13C-CA IV curves in four groups including patients with large varices, small varices, no varices, or control subjects. Significant differences were observed in averaged oral and IV curves between controls, none, small, and large varices. Results suggest the HepQuant cholate SHUNT test is detecting the presence and the size/magnitude of anatomic shunts in patients with varices.

FIG. 6B shows a graph of Compartmental model fit of the data with median adjusted R² of 0.95 for d4-CA oral curves in patients with large varices, small varices, no varices, or control subjects. Significant differences were observed in averaged oral and IV curves between controls, none, small, and large varices. Results suggest the HepQuant cholate SHUNT test is detecting the presence and the size/magnitude of anatomic shunts in patients with varices.

FIG. 6C shows a graph of the median anatomic shunt flow rate (q_{PS}) in patients with large varices, small varices, no varices, and control subjects. Significant differences were demonstrated between all groups (Wilcoxon rank-sum p<0.05).

FIG. 6D shows a graph of portal inflow to the liver (q_{PL}) in patients with large varices, small varices, no varices, and control subjects. Reduced portal inflow to the liver (q_{PL}) was observed among varices groups, all groups p<0.001.

FIG. 7 shows a graph of DSI correlation between DSI values from the Compartmental Model data fit compared to Minimal Model data fit of cholate SHUNT test data in patients with large varices, small varices, no varices, and control subjects. The Compartmental Model DSI correlated well to Minimal Model DSI-a previously validated measure of global liver function.

FIG. 8 shows a graph showing a Parameter scan of extraction ratio (ER). Simulated concentrations of 13C-CA (dotted lines) and d4-CA (solid lines) were generated with ER equal to zero. The ratio of oral d4-CA to IV 13C-CA systemic concentrations reflects the same ratio as the administered doses (2:1 oral to IV dose), demonstrating that the compartmental model is generating the expected response. The delay in reaching peak concentration is due to the cholate binding kinetics (i.e., extravascular cholate dissociating from cells/tissues and reentering plasma).

FIG. 9 shows 16 graphs of individual compartmental model fits of the HepQuant SHUNT test data (dots) for 13C-CA (dotted lines) and d4-CA (solid lines) from 3 repeated measurements (run 1=red, run 2=green, run 3=blue) for 16 control subjects. Adjusted R² values are provided for 13C-CA and d4-CA curve fits separately. Mean squared error (MSE) and Akaike Information Criterion (AIC) represent the composite of oral and IV curve fits.

FIG. 10 shows 16 graphs of individual compartmental model fits of the HepQuant SHUNT test data (dots) for 13C-CA (dotted lines) and d4-CA (solid lines) from 3 repeated measurements (run 1=red, run 2=green, run 3=blue) for 16 HCV subjects. Adjusted R² values are provided for 13C-CA and d4-CA curve fits separately. Mean squared error (MSE) and Akaike Information Criterion (AIC) represent the composite of oral and IV curve fits.

FIG. 11 shows 16 graphs of individual compartmental model fits of the HepQuant SHUNT test data (dots) for 13C-CA (dotted lines) and d4-CA (solid lines) from 3 repeated measurements (run 1=red, run 2=green, run 3=blue) for 16 NASH subjects. Adjusted R² values are provided for 13C-CA and d4-CA curve fits separately. Mean squared error (MSE) and Akaike Information Criterion (AIC) represent the composite of oral and IV curve fits.

FIG. 12 shows a comparison of Compartmental Model (CM) and Minimal Model (MM). Correlation plot (left) comparing SHUNT values from each model. Bland-Altman plot (right) shows agreement between the two methods for calculating SHUNT and 95% confidence interval for upper and lower limits of agreement (dotted lines).

FIG. 13 shows a comparison of Compartmental Model (CM) and Minimal Model (MM). Correlation plot (left) comparing HFR_{S} values from each model. Bland-Altman plot (right) show agreement between the two methods for calculating HFR_{S} and 95% confidence interval for upper and lower limits of agreement (dotted lines).

FIG. 14 shows a comparison of Compartmental Model (CM) and Minimal Model (MM). Correlation plot (left) comparing HFR_{P} values from each model. Bland-Altman plot (right) show agreement between the two methods for calculating HFR_{P} and 95% confidence interval for upper and lower limits of agreement (dotted lines).

FIG. 15 shows a comparison of Compartmental Model (CM) and Minimal Model (MM). Correlation plot (left) comparing AUC_{IV} values from each model. Bland-Altman plot (right) show agreement between the two methods for calculating AUC_{IV} and 95% confidence interval for upper and lower limits of agreement (dotted lines).

FIG. 16 shows a comparison of Compartmental Model (CM) and Minimal Model (MM). Correlation plot (left) comparing AUC_{POV} values from each model. Bland-Altman plot (right) show agreement between the two methods for calculating AUC_{PO} and 95% confidence interval for upper and lower limits of agreement (dotted lines).

FIG. 17 shows a graph of the effect of anatomic shunting on bioavailability of oral cholate dose (%) and HFR. A simulation was performed varying the portal-systemic shunt flow (q_{PS}) while holding the extraction ratio (ER) at 0.78. As chronic liver disease progresses, and the percent of shunted splanchnic flow (%) increases, portal HFR decreases dramatically in a concave fashion, systemic HFR decreases, and bioavailability increases.

FIG. 18 shows a graph of the effect of hepatic extraction on bioavailability of oral cholate dose (%) and HFR. A simulation was performed varying the hepatic extraction ratio (ER) while holding q_{PS} at 0 L/min. As chronic liver disease progresses, and the extraction ratio decreases, the portal HFR decreases dramatically in a convex fashion, systemic HFR decreases, and bioavailability increases somewhat. As shown in FIGS. 17 and 18, the parameter scan of the Compartmental Model demonstrated the ability to differentiate effects of anatomic shunting and hepatocyte function.

### DETAILED DESCRIPTION OF THE INVENTION

The cholate SHUNT test is a liver function test designed to measure clearances of orally and intravenously administered distinguishable cholates from portal and systemic circulations simultaneously.

U.S. Pat. No. 8,613,904, Everson et al., discloses cholate SHUNT test methods for evaluating liver function in a patient comprising obtaining patient serum samples following administration of two distinguishable stable isotope labeled cholate compounds, laborious sample processing and analysis of patient serum samples utilizing GC-MS.

U.S. Pat. No. 8,778,299, Everson, discloses cholate SHUNT test methods for evaluating liver function comprising obtaining patient serum samples following administration of two distinguishable stable isotope labeled cholate compounds, processing and analysis of patient serum samples utilizing HPLC-MS. Methods for determining portal hepatic filtration rate (portal HFR, FLOW) from oral administration of a distinguishable agent are also provided.

U.S. Pat. No. 9,091,701, Everson, discloses methods for determining liver function and obtaining a Disease Severity Index (DSI) value in a patient comprising obtaining patient serum samples following administration of two distinguishable stable isotope labeled cholate compounds, processing and analysis of patient serum samples utilizing HPLC-MS.

U.S. Pat. No. 8,961,925, Everson, discloses methods for performing the STAT test comprising oral administration of a distinguishable agent and measuring the distinguishable compound in a single blood or serum sample following using HLPC-MS. Methods of estimating portal hepatic filtration rate (portal HFR) from STAT values are also provided.

US Pat. Appl. Pub. No. US 2021/0318274 A1, Everson et al., discloses improved methods for blood or serum sample preparation, analyte detection and quantification which may be applied to one or more of the SHUNT, FLOW, STAT, and DSI liver function tests.

A comparison of typical embodiments of SHUNT, FLOW, STAT, and DSI tests is shown in Table 1.

TABLE 1
Liver Function Tests.

| | Exemplary | Route of | | What is |
|---|---|---|---|---|
| | Distinguishable | Admin- | | Measured or |
| Test Name | Compound | istration | Samples | Defined |
| SHUNT | ¹³C-cholate | Intra- | n = 5 | Clearances and |
| | 4D-²H-cholate | venous | over 90 | Shunt- |
| | | Oral | min | comprehensive |
| | | | | assessment of |
| | | | | hepatic blood |
| | | | | flow and hepatic |
| | | | | function using a |
| | | | | Minimal Model |
| | | | | (MM) method |
| FLOW | 4D-²H-cholate | Oral | n = 5 | Portal |
| | | | over 90 | circulation |
| | | | min | (portal hepatic |
| | | | | filtration rate; |
| | | | | Portal HFR); |
| | | | | portal HFR may |
| | | | | be estimated |
| | | | | from STAT |
| STAT | 4D-²H-cholate | Oral | n = 1 at | Estimates |
| | | | 45 min or | FLOW and |
| | | | 60 min | correlates with |
| | | | | SHUNT |
| | | | | Estimates DSI |
| DSI | ¹³C-cholate | Intra- | n = 5 | Clearances and |
| | 4D-²H-cholate | venous | over 90 | Shunt- |
| | | Oral | min | comprehensive |
| | | | | assessment of |
| | | | | hepatic blood |
| | | | | flow and hepatic |
| | | | | function; |
| | | | | DSI may also be |
| | | | | estimated from |
| | | | | STAT. |

Values for normal liver function were previously established in healthy controls in previous studies: the average SHUNT is ˜20%, average HFR (FLOW) is ˜30, and average STAT value is ˜0.4.

In the diseased liver, as more blood escapes extraction by intra- and extra-hepatic shunting to the systemic circulation, the SHUNT increases (˜30-90%), portal HFR (FLOW) or portal flow decreases (˜20 to 2 mL/min/kg), and STAT increases (0.6 to 5 uM).

A dual cholate clearance method relies on the natural clearance by the liver of the endogenous bile acid cholate (cholic acid, CA). In the dual cholate clearance test two distinguishable cholates are given to the patient, for example, each labeled with stable isotopes to distinguish them from the endogenous cholic acid naturally present. For example, the patient receives a 20 mg dose of cholic acid-24-¹³C (13C-CA) in an intravenous bolus. Simultaneously, the patient drinks a dose of 40 mg cholic-acid-2,2,4,4-d4 (4D-CA) dissolved in NaHCO₃ and mixed with juice. The cholate SHUNT test uses stable, nonradioactive 13C-cholate administered intravenously and d4 cholate administered orally, followed by serum sampling at 5, 20, 45, 60, and 90 minutes and analysis by LC/MS.

The HepQuant cholate SHUNT test has several unique attributes. It simultaneously measures hepatocyte function from the uptake of cholate and the portal circulation from the spillover of orally administered cholate, relative to intravenously administered, cholate. It is minimally invasive, blood-based, plausibly linked to the pathophysiology of all CLD, comprehensive in the assessment of hepatic function and physiology, tolerable, and is applicable to all stages of liver disease; across all etiologies, relevant populations, and clinical centers. The test is not operator-dependent and avoids need for specialized equipment or training. The results of the analyses reported in this paper support the conclusion that HepQuant SHUNT is measuring key hepatocyte and hepatic circulatory processes that determine liver health; and, therefore the health and physiologic status of the patient with CLD. Improvements due to the CM of HepQuant SHUNT could further enhance its diagnostic performance.

The DSI quantified by the HepQuant cholate SHUNT test using a noncompartmental MM calculates the combined effects of hepatic extraction and portal-systemic shunting in diseased subjects. However, the noncompartmental MM method of analysis incompletely differentiates these two contributions.

Improved methods for analysis of cholate SHUNT test data are provided herein that employ a physiological-based CM and can (1) fit clearance data from individual subjects, (2) correlate with previously validated MM, (3) improve test reproducibility, and (4) enhance the interpretation of the HepQuant cholate SHUNT test results by estimating parameters related to hepatic extraction and shunting.

In some embodiments, the disclosure provides a method for determination of compartmental model (CM) cholate SHUNT in a subject. The CM cholate SHUNT method can be used for assessment of liver function, the progression of at least one hepatic condition, treatment efficacy, need for treatment, and/or likelihood of a clinical outcome in a subject.

A method is provided for determining a compartmental model cholate SHUNT value in a subject in need thereof, comprising
obtaining blood or serum concentration data of an orally administered first distinguishable cholate compound in samples collected from the subject at a multiplicity of time points after administration;
obtaining blood or serum concentration data of an intravenously administered second distinguishable cholate compound in the samples collected from the subject at the multiplicity of time points after administration;
fitting the first and second distinguishable cholate concentration data to a physiological-based compartmental model of dual cholate clearance to obtain fitted data, the compartmental model comprising body mass index (BMI), body weight (BW), and hematocrit (Hct) input values in the subject; and
calculating one or more indices of hepatic disease in the subject using the fitted data, wherein the one or more indices is associated with liver function in the subject.

The CM cholate SHUNT value can be used for assessment of liver function, progression of at least one hepatic condition, treatment efficacy, need for treatment, and/or likelihood of a clinical outcome in a subject. In some embodiments, the CM cholate SHUNT value may be used for assessment of liver function in the subject.

A method is provided for determining liver function in a subject, the method comprising:
receiving a plurality of blood or serum samples collected form the subject over time intervals for a period of less than 3 hours after administering orally a first distinguishable cholate; and co-administering intravenously a second distinguishable cholate to the subject;
quantifying the concentration of the first and the second distinguishable cholates in the samples to obtain first and second distinguishable cholate concentration data;
fitting the first and second distinguishable cholate concentration data to a physiological-based compartmental model of dual cholate clearance to obtain fitted concentration data, the compartmental model comprising body mass index (BMI), body weight (BW), and hematocrit (Hct) input values in the subject; and
calculating one or more indices of hepatic disease in the subject using the fitted data, wherein the one or more indices is associated with liver function in the subject.

A method is provided for determining liver function in a subject, the method comprising:
administering orally a first distinguishable cholate to a subject having, or suspected of having or developing, a hepatic disorder; and co-administering intravenously a second distinguishable cholate to the subject;
collecting a multiplicity of blood or serum samples over time intervals for a period of less than 3 hours after administration of the agents to the subject;
quantifying the concentration of the first and the second distinguishable cholates in the samples to obtain first and second distinguishable cholate concentration data;
fitting the first and second distinguishable cholate concentration data to a physiological-based compartmental model of dual cholate clearance to obtain fitted data; and
calculating one or more indices of hepatic disease in the subject using the fitted data, wherein the one or more indices is associated with liver function in the subject.

The one or more indices of hepatic disease may be selected from cholate SHUNT, DSI, HR, HFRs, HFRp, and STAT.

The cholate SHUNT may be calculated using the formula: AUCoral/AUCiv x Doseiv/Doseoral×100% using the fitted first and second distinguishable cholate concentration concentration data; wherein AUCoral is the area under the curve of the fitted serum concentrations of the first cholate and AUCiv is the area under the curve of the of the fitted serum concentrations of the second cholate. The cholate SHUNT value may be an indicator of liver function, the presence or progression of at least one hepatic condition, treatment efficacy, need for treatment, and/or likelihood of a clinical outcome in the subject.

The orally administering of the first labeled cholate and the intravenously co-administering of the second labeled cholate may be performed simultaneously. In some aspects, the collecting step comprises collecting a multiplicity of samples over a period of about 90 minutes or less. In some aspects, the samples comprise blood or serum samples collected from the subject at 5, 20, 45, 60, and 90 minutes post-administration. In some aspects, the quantifying of the concentration of the first and the second distinguishable cholates in the blood or serum samples may be performed by any method known in the art. For example, the quantifying of the concentration of the first and the second distinguishable cholates in the samples may comprise GC-MS, HPLC-MS, or LC-MS/MS analytical techniques.

In some embodiments, the physiological-based compartmental model comprises mathematical terms describing compartment volumes of a multiplicity of compartments in the subject; flow parameters between the multiplicity of compartments in the subject; and hepatic extraction ratios of the orally and intravenously administered distinguishable cholates in the subject. The physiological-based compartmental model may further comprise mathematical terms describing cholate binding in the subject.

### Definitions

As used herein, “a” or “an” may mean one or more than one of an item.

The term “about” when referring to any numerical parameter means +/−10% of the numerical value. For example, the phrase “about 60 minutes” refers to 60 minutes +/−6 minutes.

All patents, patent applications and publications referred to herein are incorporated by reference herein in their entirety.

The term “accuracy” (measurement) when used herein refers to closeness of agreement between a measured quantity value and a true quantity value of a measurand.

The term “acceptability” as used herein is based on individual criteria that set minimal operational characteristics for a measurement procedure.

The term “precision” as used herein refers to closeness of agreement between independent test/measurement results obtained under stipulated conditions.

The term “trueness” as used herein refers to the closeness of agreement between the expectation of a test result or a measurement result and a true value.

The term “measureand” is used when referring to the quantity intended to be measured instead of analyte (component represented in the name of a measurable quantity).

The term “verification” as used herein focuses on whether specifications of a measurement procedure can be achieved, whereas the term “validation” verifies that the procedure is fit for an intended purpose.

The term “measurement procedure” refers to a detailed description of a measurement according to one or more measurement principles and to a given measurement method, based on a measurement model and including any calculation to obtain a measurement result.

As used herein “clearance” may mean the removing of a substance from one place to another.

As used herein, the term “simultaneously” when referring to 2 or more events refers to occurring within 20 minutes or less, within 15 minutes, 10 minutes, 5 minutes, or within about 3 minutes of each other.

As used herein the terms, “patient”, “subject” or “subjects” include but are not limited to humans, the term may also encompass other mammals, or domestic or exotic animals, for example, dogs, cats, ferrets, rabbits, pigs, horses, cattle, birds, or reptiles.

The acronym “HALT-C” refers to the Hepatitis C Antiviral Long-term Treatment against Cirrhosis trial. The HALT-C trial was a large, prospective, randomized, controlled trial of long-term low dose peg interferon therapy in patients with advanced hepatitis C who had not had a sustained virologic response to a previous course of interferon-based therapy. An NIH-sponsored Hepatitis C Antiviral Long-Term Treatment against Cirrhosis (HALT-C) Trial examined whether long-term use of antiviral therapy (maintenance treatment) would slow the progression of liver disease. In noncirrhotic patients who exhibited significant fibrosis, effective maintenance therapy was expected to slow or stop histological progression to cirrhosis as assessed by serial liver biopsies. However, tracking disease progression with biopsy carries risk of complication, possibly death. In addition, sampling error and variation of pathologic interpretation of liver biopsy limits the accuracy of histologic assessment and endpoints. The histologic endpoint is less reliable because advanced fibrosis already exists and changes in fibrosis related to treatment or disease progression cannot be detected. Thus, standard endpoints for effective response to maintenance therapy in cirrhotic patients are prevention of clinical decompensation (ascites, variceal hemorrhage, and encephalopathy) and stabilization of liver function as measured clinically by Childs-Turcotte-Pugh (CTP) score. However, clinical endpoints and CTP score were known to be insensitive parameters of disease progression. Dual isotope techniques employing distinguishable cholates were used in development of the SHUNT test and used in conjunction with the HALT-C trial. The term “SHUNT test” refers to a previously disclosed QLFT (quantitative liver function test) used as a comprehensive assessment of hepatic blood flow and liver function. The SHUNT test is used to determine clearance of orally and intravenously administered distinguishable cholic acids in subjects with and without chronic liver disease. SHUNT fraction or percent quantifies the spillover of the PO d4-cholate into the systemic circulation from the ratio of the clearance of the intravenously administered 13C-cholate to the clearance of the orally administered d4-cholate. In the SHUNT test, at least 5 blood samples are analyzed which have been drawn from a patient at intervals over a period of at least about 90 minutes after oral and intravenous administration of differentiable cholates. The SHUNT test is disclosed in Everson et al., U.S. Pat. No. 8,613,904, which is incorporated herein by reference. These studies demonstrated reduced clearance of cholate in patients who had either hepatocellular damage or portosystemic shunting. The “SHUNT test value” refers to a number (in %). The term “SHUNT %” represents a quantitative measurement of portal-systemic shunting. SHUNT % is a measurement of the percentage of spillover of the orally administered d4-cholate. The first-pass hepatic elimination of cholate in percent of orally administered cholate is defined as (100%−SHUNT). SHUNT test methods are disclosed in U.S. Pat. Nos. 8,613,904, 9,639,665, 8,778,299, 9,417,230, and 10,215,746, each of which is incorporated herein by reference in its entirety. Analysis of samples for stable isotopically labeled cholates is performed by, e.g., GC-MS, following sample derivitization, or LC-MS, without sample derivitization, or LC-MS/MS, or MS/MS as disclosed herein. The ratio of the AUCs of orally to intravenously administered cholic acid, corrected for administered doses, defines cholate shunt. The cholate shunt can be calculated using the formula: AUCₒᵣₐₗ/AUCᵢᵥ×Doseᵢᵥ/Doseₒᵣₐₗ×100%, wherein AUCₒᵣₐₗ is the area under the curve of the serum concentrations of the orally administered cholic acid and AUCᵢᵥ is the area under the curve of the intravenously administered cholic acid.

The SHUNT test allows measurement of first-pass hepatic elimination of bile acids from the portal circulation. Flow-dependent, first pass elimination of bile acids by the liver ranges from about 60% for unconjugated dihydroxy, bile acids to about 95% for glycine-conjugated cholate. Free cholate, used herein has a reported first-pass elimination of approximately 80% which agrees closely with previously observed first pass elimination in healthy controls of about 83%. After uptake by the liver, cholic acid is efficiently conjugated to either glycine or taurine and secreted into bile. Physicochemically cholic acid may be easily separated from other bile acids and bile acid or cholic acid conjugates, using chromatographic methods.

The term “Cholate Elimination Rate”, kₑₗᵢₘ min⁻¹ represents the first phase of elimination of the intravenously administered 13C-cholate, calculation from Ln/linear regression of [13C-cholate] versus time (using only the 5- and 20-minute time points). Intravenously administered 13C-cholate is rapidly delivered to the liver via the hepatic artery. In contrast, the same 13C-cholate slowly transits to the liver via the portal vein due to the capacitance of the splanchnic vascular bed. Thus, the first phase of cholate elimination is more dependent upon clearance from the hepatic artery than from portal vein.

The term “Volume of distribution”, V_{d}, L kg⁻¹ represents the body's volume into which cholate is distributed. This is calculated from the intercept on the Y axis of the Ln/linear regression of [13C-cholate] versus time (using only the 5- and 20-min time points).

The acronym “IV” or “iv” refers to intravenous route of administration.

The acronym “PO” refers to per oral route of administration.

The acronym “PHM” refers to perfused hepatic mass.

The acronym “SF” refers to shunt fraction, for example, as in liver SF, or cholate SF.

The acronym “ROC” refers to receiver operating characteristic. The ROC curve is a graphical plot which illustrates performance of a binary classifier system as its discrimination threshold is varied. It is created by plotting the fraction of true positives out of the positives (TPR=true positive rate) vs. the fraction of false positives out of the negatives (FPR=false positive rate), at various threshold settings. Sensitivity is the probability of a positive test result, or of a value above a threshold, among those with disease. Sensitivity is defined as the true positive rate (TPR): TPR=TP/P=TP/(TP+FN). False positive rate (FPR) is FPR=FP/N=FP/(FP+FN). Accuracy (ACC) is defined as ACC=(TP+TN)/(P+N). Specificity is the probability of a negative test result, or a value below a threshold, among those without disease. Specificity (SPC), or true negative rate (TN) is defined as SPC=TN/N=TN/(FP+TN)=1−FPR. Positive prediction value (PPV) is defined as: PPV=TP/(TP+FP). Negative predictive value (NPV) is defined as NPV=TN/(TN+FN).

The c-statistic is the area under the ROC curve, or “AUROC” (area under receiver operating characteristic curve) and ranges from 0.5 (no discrimination) to a theoretical maximum of 1 (perfect discrimination).

The terms “treating” or “treatment” of a disease state or condition includes: (i) preventing the disease state or condition, i.e., causing the clinical symptoms of the disease state or condition not to develop in a subject that may be exposed to or predisposed to the disease state or condition, but does not yet experience or display symptoms of the disease state or condition, (ii) inhibiting the disease state or condition, i.e., arresting the development of the disease state or condition or its clinical symptoms, or (iii) relieving the disease state or condition, i.e., causing temporary or permanent regression of the disease state or condition or its clinical symptoms.

The term “sustained virologic response” (SVR) is used to describe a desired response in a patient when, e.g., hepatitis C virus is undetectable in the blood six months after finishing treatment. Conventional treatment using interferon and ribavirin doesn't necessarily eliminate, or clear, the hepatitis C virus. A sustained virologic response is associated with a very low incidence of relapse. SVR is used to evaluate new medicines and compare them with proven therapies.

The term “distinguishable cholate” may be selected from any of the following labeled compounds: cholic acid, any glycine conjugate of cholic acid, any taurine conjugate of cholic acid; chenodeoxycholic acid, any glycine conjugate of chenodeoxycholic acid, any taurine conjugate of chenodeoxycholic acid; deoxycholic acid, any glycine conjugate of deoxycholic acid, any taurine conjugate of deoxycholic acid; or lithocholic acid, or any glycine conjugate or taurine conjugate thereof. The distinguishable cholate compounds may be isotope labeled cholate compounds. Distinguishable cholate compounds may be labeled with either stable (e.g., ¹³C, ²H, ¹⁸O) or radioactive (e.g., ¹⁴C, ³H) isotopes. Distinguishable cholate compounds can be purchased (for example CDN Isotopes Inc., Quebec, CA).

The distinguishable cholate compounds may be stable isotope labeled cholate compounds. The distinguishable cholate may be selected from any known safe, non-radioactive stable isotope of cholic acid. In one specific aspect, the distinguishable cholate compound is 2,2,4,4-²H cholic acid, also known as cholic-acid-2,2,4,4-d₄ (D₄-CA). In another specific aspect, the distinguishable cholate compound is 24-¹³C cholic acid, also known as cholic acid-24-¹³C (¹³C-CA). In another specific aspect, the distinguishable compound is 2,2,3,4,4-²H cholic acid, also known as cholic acid-2,2,3,4,4-d₅ (D₅-CA).

Cholates occur naturally and are not known to have any deleterious or adverse effects when given intravenously or orally in the doses used in HQ tests. The serum cholate concentrations that are achieved by either the intravenous or oral doses are similar to the serum concentrations of bile acids that occur after the ingestion of a fatty meal. Because cholates are naturally occurring with a pool size in humans of 1 to 5 g, the 20 and 40 mg doses of labeled cholates used herein are unlikely to be harmful.

The term “oral cholate clearance” (Clₒᵣₐₗ) refers to clearance from the body of a subject of an orally administered cholate compound as measured by a blood or serum sample from the subject. Oral cholate clearance is used as a measure of portal blood flow. Orally administered cholic acid is absorbed across the epithelial lining cells of the small intestine, bound to albumin in the portal blood, and transported to the liver via the portal vein. Approximately 80% of cholic acid is extracted from the portal blood in its first pass through the liver. Cholic acid that escapes hepatic extraction exits the liver via hepatic veins that drain into the vena cava back to the heart, and is delivered to the systemic circulation. The area under the curve (AUC) of peripheral venous concentration versus time after oral administration of cholic acid quantifies the fraction of cholic acid escaping hepatic extraction and defines “oral cholate clearance”.

The term “portal hepatic filtration rate”, “portal HFR”, “FLOW test” (HFRp) refers to oral cholate clearance (portal hepatic filtration rate; portal HFR) used as a measure of portal blood flow, or portal circulation, obtained from analysis of concentration of distinguishable cholate compound in at least 5 blood samples drawn from a subject over a period of, for example, about 90 minutes after oral administration of a distinguishable cholate compound, for example, a distinguishable cholate. The units of portal HFR value are typically expressed as mL/min/kg, where kg refers to kg body weight of the subject. “Portal HFR”, mL min⁻¹ kg⁻¹ may be used to Model independent apparent clearance of orally administered d4-cholate, adjusted for body weight, and calculated from dose/AUC. FLOW test methods are disclosed in U.S. Pat. Nos. 8,778,299, 9,417,230, and 10,215,746, each of which is incorporated herein by reference in its entirety.

The term “Systemic HFR”, (HFRs) mL min⁻¹ kg⁻¹ may be used to Model independent clearance of intravenously injected 13C-cholate, adjusted for body weight, and calculated from dose/AUC. The “Systemic HFR”, mL min⁻¹ kg⁻¹, may be used to Model independent clearance of intravenously injected 13C-cholate, adjusted for body weight, and calculated from dose/AUC.

The term “STAT test” (STAT) refers to an estimate of portal blood flow by analysis from one patient blood sample drawn at a defined period of time following oral administration of a differentiable cholate. In one aspect, the STAT test refers to analysis of a single blood sample drawn at a specific time point after oral administration of a differentiable cholate. In one specific aspect, the STAT test is a simplified convenient test intended for screening purposes that can reasonably estimate the portal blood flow (estimated flow rate) from a single blood sample taken 60 minutes after orally administered deuterated-cholate. In some embodiments, STAT, is the d4-cholate concentration in the 60 minute blood sample. STAT correlates well with DSI and can be used to estimate DSI. The STAT test value is typically expressed as a concentration, for example, micromolar (uM) concentration. STAT test methods are disclosed in U.S. Pat. Nos. 8,961,925, 10,222,366, each of which is incorporated herein by reference in its entirety. STAT test value may be used to estimate portal HFR, as provided in U.S. Pat. Nos. 8,961,925, 10,222,366. A STAT test value in a patient may be used to estimate a DSI value in a patient, as provided herein.

The term “DSI test” (DSI) refers to Disease Severity Index test which is derived from one or more liver function test results based on hepatic blood flow. The DSI score is a function of the sum of cholate clearances from systemic and portal circulations adjusted to disease severity ranging from healthy subjects to end stage liver disease. DSI is a score without units representing a quantitative measurement of liver function. A disease severity index (DSI) value may be obtained in a patient by a method comprising (a) obtaining one or more liver function test values in a patient having or at risk of a chronic liver disease, wherein the one or more liver function test values are obtained from one or more liver function tests selected from the group consisting of SHUNT, portal hepatic filtration rate (portal HFR), and systemic hepatic filtration rate (systemic HFR); and (b) employing a disease severity index equation (DSI equation) to obtain a DSI value in the patient, wherein the DSI equation comprises one or more terms and a constant to obtain the DSI value, wherein at least one term of the DSI equation independently represents a liver function test value in the patient, or a mathematically transformed liver function test value in the patient from step; and the at least one term of the DSI equation is multiplied by a coefficient specific to the liver function test. DSI is an index, or score, that encompasses the cholate clearances from both systemic and portal circulations. DSI has a range from 0 (healthy) to 50 (severe end-stage disease) and is calculated from both HFRs. Based on the reproducibility of DSI, the minimum detectable difference indicating a change in liver function in a subject may be about 1.5 points, about 2 points, or about 3 points. DSI test methods and equations are disclosed in U.S. Pat. Nos. 9,091,701, 9,759,731, 10,520,517, each of which is incorporated herein by reference in its entirety. Additional DSI equations have been developed and are provided herein. A method of estimating a DSI value in a patient from a STAT test value is also provided herein.

The term “Hepatic Reserve” refers to percentage of maximum hepatic functional capacity measured by DSI, indexed hepatic reserve may be normalized to the DSI range in subjects of lean body mass. HR (algebraic) is simply an algebraic conversion of the DSI value in the subject: HR=[100−(2×DSI)]. Indexed HR, is normalized against the results within a cohort of normal lean controls.

The term “RCA20” represents the amount of the intravenously administered distinguishable compound, for example, a distinguishable cholate compound such as 13C-CA, that remains in the circulation 20 minutes after the intravenous injection.

The term “Quantitative Liver Function Test” (QLFT), refers to assays that measure the liver's ability to metabolize or extract test compounds, can identify patients with impaired hepatic function at earlier stages of disease, and possibly define risk for cirrhosis, splenomegaly, and varices. One of these assays is the cholate shunt assay where the clearance of cholate is assessed by analyzing bodily fluid samples after exogenous cholate has been taken up by the body.

The term “Ishak Fibrosis Score” is used in reference to a scoring system that measures the degree of fibrosis (scarring) of the liver, which is caused by chronic necroinflammation. A score of 0 represents no fibrosis, and 6 is established fibrosis. Scores of 1 and 2 indicate mild degrees of portal fibrosis; stages 3 and 4 indicate moderate (bridging) fibrosis. A score of 5 indicates nodular formation and incomplete cirrhosis, and 6 is definite cirrhosis.

The term “Childs-Turcotte-Pugh (CTP) score” or “Child-Pugh score” refers to a classification system used to assess the prognosis of chronic liver disease as provided in Pugh et al., Transection of the oesophagus for bleeding oesophageal varices. Br J Surg 1973; 60:646-649, which is incorporated herein by reference. The CTP score includes five clinical measures of liver disease; each measure is scored 1-3, with 3 being the most severe derangement. The five scores are added to determine the CTP score. The five clinical measures include total bilirubin, serum albumin, prothrombin time international normalized ratio (PT INR), ascites, and hepatic encephalopathy. The CTP score is one scoring system used in stratifying the seriousness of end-stage liver disease. Chronic liver disease is classified into Child-Pugh class A to C, employing the added score. Child-Pugh class A refers to CTP score of 5-6. Child-Pugh class B refers to CTP score of 7-9. Child-Pugh class C refers to CTP score of 10-15. A website calculates post-operative mortality risk in patients with cirrhosis. http://mayoclinic.org/meld/mayomodel9.html

The term “Model for End-Stage Liver Disease (MELD) refers to a scoring system used to assess the severity of chronic liver disease. MELD was developed to predict death within three months of surgery in patients who had undergone a transjugular intrahepatic portosystemic shunt (TIPS) procedure patients for liver transplantation. MELD is also used to determine prognosis and prioritizing for receipt of a liver transplant. The MELD uses a patient's values for serum bilirubin, serum creatinine, and international normalized ratio for prothrombin time (INR) to predict survival. The scoring system is used by the United Network for Organ Sharing (UNOS) and Eurotransplant for prioritizing allocation of liver transplants instead of the older Child-Pugh score. See UNOS (2009 Jan. 28) “MELD/PELD calculator documentation”, which is incorporated herein by reference. For example, in interpreting the MELD score in hospitalized patients, the 3 month mortality is: 71.3% mortality for a MELD score of 40 or more.

The term “standard sample” refers to a sample with a known concentration of an analyte used for comparative purposes when analyzing a sample containing an unknown concentration of analyte.

The term “Chronic Hepatitis C” (CHC) refers to a chronic liver disease caused by viral infection and resulting in liver inflammation, damage to the liver and cirrhosis. Hepatitis C is an infection caused by a blood-borne virus that attacks the liver and leads to inflammation. Many people infected with hepatitis C virus (HCV) do not exhibit symptoms until liver damage appears, sometimes years later, during routine medical tests.

The term “Alcoholic SteatoHepatitis” (ASH) refers to a chronic condition of inflammation of the liver which is caused by excessive drinking. Progressive inflammatory liver injury is associated with long-term heavy intake of ethanol and may progress to cirrhosis.

The term “Non-Alcoholic SteatoHepatitis” (NASH) refers to a serious chronic condition of liver inflammation, progressive from the less serious simple fatty liver condition called steatosis. Simple steatosis (alcoholic fatty liver) is an early and reversible consequence of excessive alcohol consumption. In people that don't drink much alcohol, the cause of fatty liver disease is less clear, but may be associated with factors such as obesity, high blood sugar, insulin resistance, or high levels of blood triglycerides. In certain cases the fat accumulation can be associated with inflammation and scarring in the liver. This more serious form of the disease is termed non-alcoholic steatohepatitis (NASH). NASH is associated with a much higher risk of liver fibrosis and cirrhosis than NAFLD. Patients with NASH have increased risk for hepatocellular carcinoma. NAFLD may progress to NASH with fibrosis cirrhosis and hepatocellular carcinoma.

The term “Non-Alcoholic Fatty Liver Disease” (NAFLD) refers to a common chronic liver disease characterized in part by a fatty liver condition with associated risk factors of obesity, metabolic syndrome, and insulin resistance. Both NAFLD and NASH are often associated with obesity, diabetes mellitus and asymptomatic elevations of serum ALT and gamma-GT. Ultrasound monitoring can suggest the presence of a fatty infiltration of the liver; differentiation between NAFLD and NASH, typically requires a liver biopsy.

The term “Primary Sclerosing Cholangitis” (PSC) refers to a chronic liver disease caused by progressive inflammation and scarring of the bile ducts of the liver. Scarring of the bile ducts can block the flow of bile, causing cholestasis. The inflammation can lead to liver cirrhosis, liver failure and liver cancer. Chronic biliary obstruction causes portal tract fibrosis and ultimately biliary cirrhosis and liver failure. The definitive treatment is liver transplantation. Indications for transplantation include recurrent bacterial cholangitis, jaundice refractory to medical and endoscopic treatment, decompensated cirrhosis and complications of portal hypertension (PHTN). PSC progresses through chronic inflammation, fibrosis/cirrhosis, altered portal circulaton, portal hypertension and portal-systemic shunting to varices-ascites and encephalopathy. Altered portal flow is an indication of clinical complications.

The following abbreviations are employed in the present disclosure. 13C-CA=carbon-13-labeled cholate; AIC=Akaike Information Criterion; BMI=body mass index; CI=confidence interval; CLD=chronic liver disease; CM=compartmental model; d4-CA=deuterium-labeled cholate; DSI=disease severity index; ER=extraction ratio; HCV=hepatitis C virus; HFR=hepatic filtration rate; HR=hepatic reserve; ICC=intraclass correlation coefficient; IV=intravenous; LC/MS=liquid chromatography/mass spectrometry; MM=minimal model; MSE=mean squared error; NAFLD=nonalcoholic fatty liver disease; NASH=non-alcoholic steatohepatitis; TBV=total blood volume.

### Compartmental Model Development

A “compartmental model” (CM) is a pharmacokinetic technique that models the human body into one or more compartments of different volumes in which drug concentrations are described by a series of kinetic equations simulating the transfer of flow from one compartment to another. In contrast, noncompartmental analyses are model-independent and use algebraic equations to estimate pharmacokinetic parameters. Previously, a noncompartmental analysis, or minimal model (MM), was used to characterize intravenous (IV) clearance by a tri-exponential fit and oral clearance by a cubic spline fit [Everson G T, Martucci M A, Shiffman M L, Sterling R K, Morgan T R, Hoefs J C, et al. Portal-systemic shunting in patients with fibrosis or cirrhosis due to chronic hepatitis C: the minimal model for measuring cholate clearances and shunt. Aliment Pharmacol Ther. 2007; 26:401-10]. The present disclosure provides a physiological-based compartmental model (CM) that could improve diagnostic performance and enhance the interpretation of the HepQuant SHUNT test.

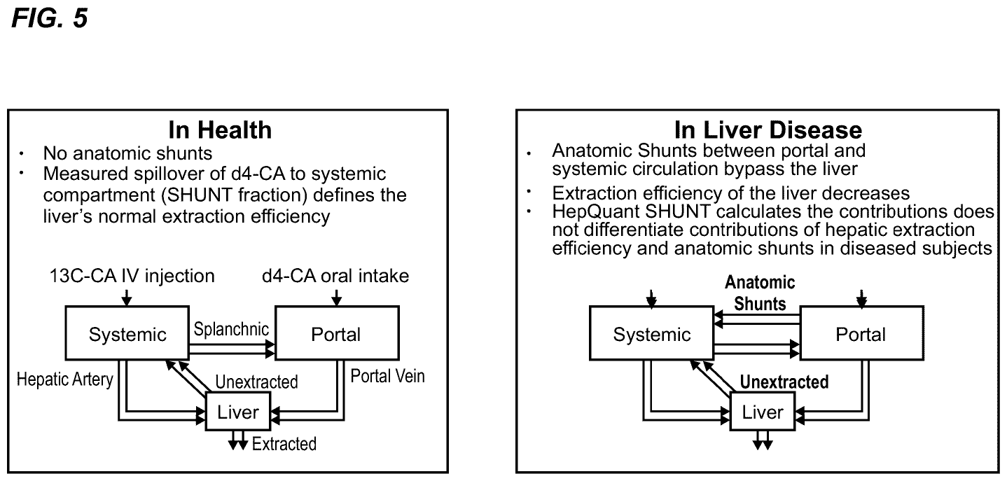

In health, the measured spillover of d4-CA to the systemic compartment defines the livers normal extraction efficiency. In health, there are no anatomic shunts, and measured spillover of the 4d-CA to the systemic compartment (SHUNT fraction) defines the liver's normal extraction efficiency as illustrated in FIG. 5, left panel.

In liver disease, impaired hepatocyte function and portal-systemic shunting causes excess cholate to flow back into systemic circulation. Anatomic shunts between portal and systemic circulation bypass the liver, and the extraction efficiency of the liver decreases, as illustrated in FIG. 5, right panel. The cholate SHUNT test using a minimal model (MM) approach calculates the contributions but does not differentiate the contributions of hepatic extraction efficiency and anatomic shunts in diseased subject.

One objective of the present disclosure is to provide a compartmental model (CM) method of analyzing cholate SHUNT test data in order to estimate the relative contributions of anatomic shunting and hepatic extraction.

In the present disclosure, a new physiologically based compartmental model (CM) is provided for evaluation of portal and systemic clearance of distinguishable cholates. The CM describes transfer of cholates between systemic, portal, and liver compartments with assumptions from measured or literature-derived values and unknown parameters estimated by nonlinear least-squares regression.

In CM method development, data from a previous study of HepQuant cholate SHUNT test reproducibility were analyzed retrospectively [Burton et al., Transl Res. 2021; 233:5-15]. The study consisted of 48 subjects from three groups: controls (N=16), NASH (N=16), and hepatitis C virus (HCV) (N=16). Controls were healthy persons with no history of liver disease and normal standard blood tests. NASH was diagnosed based on risk factors (obesity, diabetes, metabolic syndrome), negative tests for other liver disease, and fibrosis stage by liver biopsy or transient elastography. HCV was diagnosed based on a history of positive HCV by nucleic acid testing and METAVIR fibrosis stage by liver biopsy. Three replicate HepQuant SHUNT tests were conducted on three separate days within 30 days.

### Compartmental Model

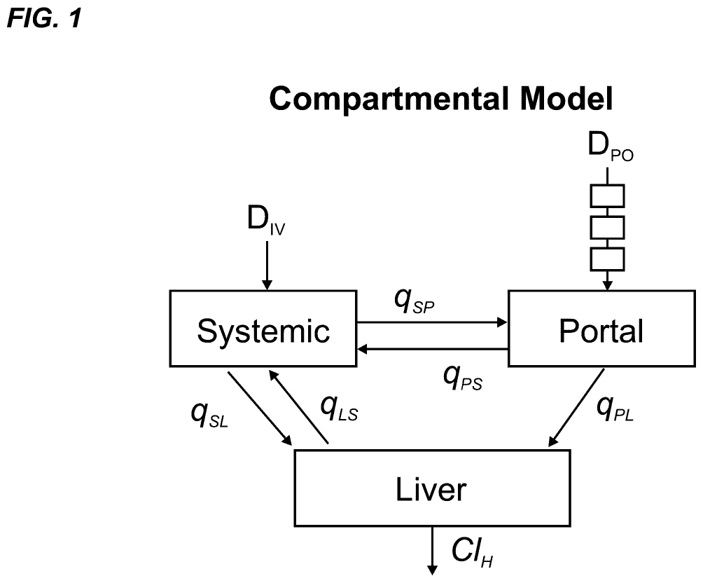

A CM was developed to describe dual cholate clearance in the HepQuant SHUNT test (FIG. 1) and is divided into three main compartments: the systemic compartment (S), the portal compartment (P), and the liver (L). At time 0, a 20mg dose of 13C-CA is administered intravenously and a 40 mg dose of d4-CA is administered orally. The systemic compartment has two output flows: one to the portal compartment via splanchnic arterial circulation (q_{SP}) and another to the liver via the hepatic artery (q_{SL}).

In healthy individuals, all cholate from the portal compartment flows into the liver compartment, and the hepatic extraction is approximately 75%. In liver disease, two conditions may reduce the total hepatic clearance: (1) collateral circulation and shunting between portal and systemic compartments reduce inflow to the liver and (2) hepatocyte extraction decreases cholate uptake. The following sections detail the CM and provide rationale for various aspects of the model.

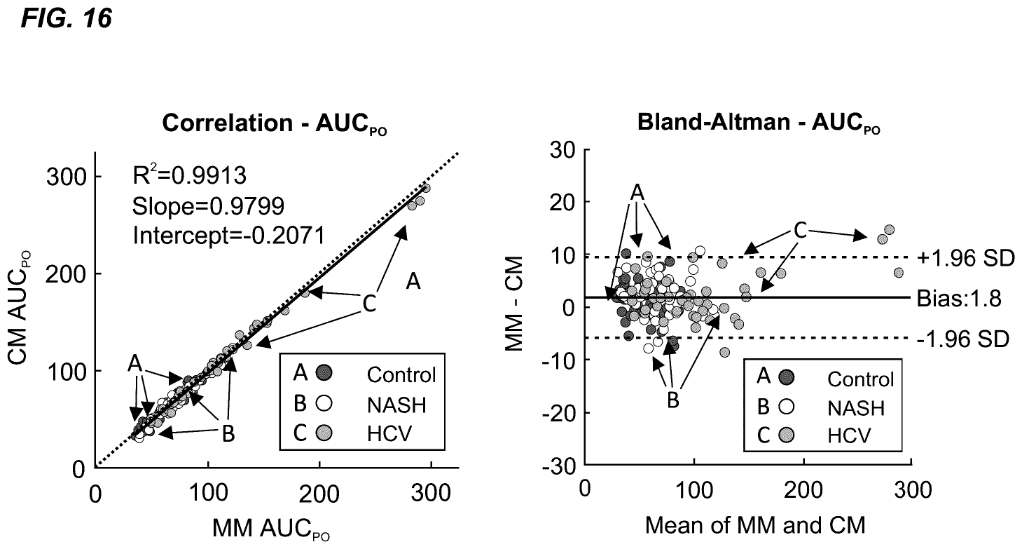

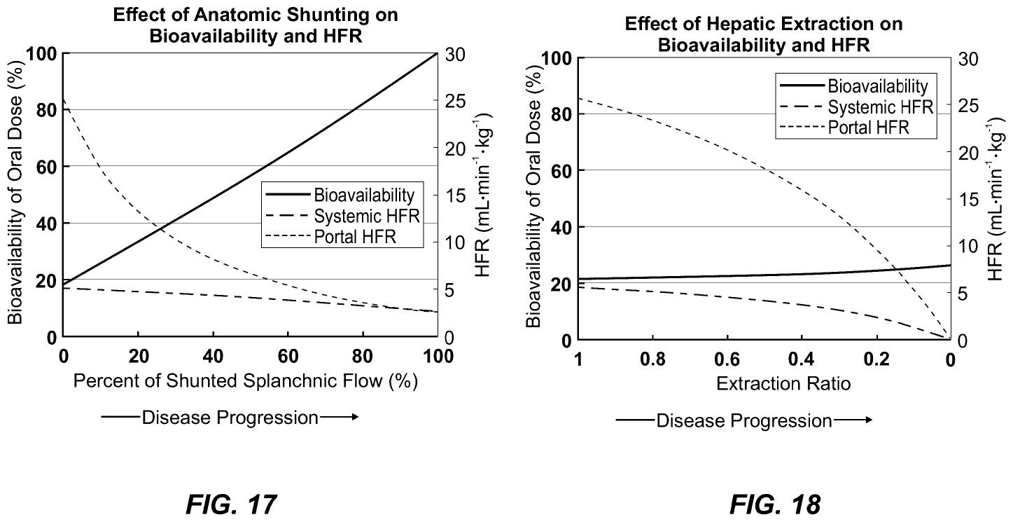

One advantage of the Compartmental Model is the ability to differentiate effects of anatomic shunting and hepatocyte function. A simulation was performed varying the portal-systemic shunt flow (q_{PS}) while holding the extraction ratio (ER) at 0.78. FIG. 17 shows a graph of the effect of anatomic shunting on bioavailability of oral cholate dose (%) and HFR. As chronic liver disease progresses, and the percent of shunted splanchnic flow (%) increases, portal HFR decreases dramatically in a concave fashion, systemic HFR decreases, and bioavailability increases. Another simulation was performed varying the hepatic extraction ratio (ER) while holding qps at 0 L/min. FIG. 18 shows a graph of the effect of hepatic extraction on bioavailability of oral cholate dose (%) and HFR. As chronic liver disease progresses, and the extraction ratio decreases, the portal HFR decreases dramatically in a convex fashion, systemic HFR decreases, and bioavailability increases somewhat. As shown in FIGS. 17 and 18, the parameter scan of the Compartmental Model demonstrated the ability to differentiate effects of anatomic shunting and hepatocyte function.

### Compartment Volumes

Total blood volume (TBV) was calculated by Equation 1 which accounts for the nonlinear relationship between blood volume and body mass index (BMI, kg/m²) across the entire range of body weights (BW) including obese (BMI 30-40) and morbidly obese (BMI>40) subjects [15].

$$TBV = \frac{0.07 \cdot BW}{\sqrt{BMI / 22}} \qquad ( 1 )$$

In this equation, 22 is the BMI value corresponding to ideal body weight, and 0.07 is the indexed blood volume (in L·kg⁻¹) for a subject with a BMI of 22. Whole blood is comprised of approximately 55% plasma and 45% hematocrit [16]. A plasma fraction (fₚₗₐₛₘₐ) was used for adjusting volumes according to the fraction of whole blood without red blood cells (i.e., 1 minus hematocrit) to approximate the serum sampling used in the HepQuant SHUNT test. The systemic compartment volume (V_{S}) was estimated by multiplying TBV by fₚₗₐₛₘₐ.

It is estimated that the liver receives 25% of the total cardiac output [17, 18]. Thus, the volume of the portal compartment (V_{P}) was approximated to be 25% of the systemic volume. To calculate the volume of the liver compartment (V_{L}), liver volumes corrected for body weight from 13 studies of ultrasound and computerized tomography scans were averaged (22.46±1.98 mL·kg⁻¹) and multiplied by BW and liver tissue density (d_{L}) to obtain liver weight (w_{L}). Liver tissue density of 1.06 g·mL⁻¹ was derived from the average of two studies estimating liver densities of 1.04 g·mL⁻¹ [20] and 1.08 g·mL⁻¹ [21]. Hepatic blood volume ranges from 25 to 30 mL per 100 g liver weight [17, 22]. Using the average of this range (0.275 mL·g⁻¹), V_{L} (in liters) was estimated by Equation 2.

V_{L}=(0.275·22.46·BW·d_{L}·fₚₗₐₛₘₐ)/1000 (2)

### Flow Parameters

The cholate concentration measured by the HepQuant cholate SHUNT test is only the albumin-bound and free cholate found in serum; thus, the concentration of extravascular cholate bound/distributed to cells and tissues (“BIV” and “BPO”) does not contribute to the measured concentration when fitting the data.

Total hepatic flow rate (Q_{L}) was comprised of both hepatic arterial (q_{SL}) and portal venous (q_{PL}) inflows to the liver (Equation 3).

Q_{L}=q_{SL}+q_{PL} (3)

Using the previous estimate for liver weight, w_{L}, the initial estimate for total hepatic flow rate (Q_{L, init}) was approximately 1 L·min⁻¹·kg⁻¹ liver wet weight [17, 23].

Splanchnic circulation (q_{SP}) is defined as the blood flow to the abdominal gastrointestinal organs, including the stomach, liver, spleen, pancreas, and intestines. A variety of mechanisms for splanchnic blood flow control have been proposed in which the splanchnic vascular bed functions with an autoregulatory capacity to maintain a constant blood flow across a

range of perfusion pressures [24]. For the purpose of this model, q_{SP} was assumed to be proportional to the total cardiac output. Since splanchnic circulation accounts for approximately 75% of the total liver inflow (with the remaining 25% supplied by the hepatic artery) [17], q_{SP} was defined as a constant flow rate at 75% of the total liver inflow (Q_{L}).

The liver's unique dual blood supply draws from both the hepatic artery and the portal vein. Hepatic arterial inflow (q_{SL}) accounts for about 25% of the total inflow to the liver [17], is highly adaptable [25], and is capable of compensating for changes in portal venous flow with studies suggesting that 25%-60% of decreased portal flow can be buffered by the hepatic artery [26, 27]. Thus, q_{SL} was modeled as an adaptive flow rate with an initial estimate q_{SL, init} of 25% of the total inflow to the liver (Q_{L}).

In health, the portal venous inflow to the liver (q_{PL}) equals the splanchnic arterial flow rate (q_{SP}). However, in the presence of collateral circulation (e.g., portosystemic shunts or esophageal and gastric varices), portal venous inflow is reduced by the shunt flow (q_{PS}) which bypasses the liver (Equation 4).

q_{PL}=q_{SP}−q_{PS} (4)

Lastly, the total hepatic venous return flow to systemic circulation (q_{LS}) is equal to the total inflow to the liver, Q_{L}.

### Flow Parameters

A system of differential equations describes the 13C-CA and d4-CA concentrations in the systemic (C_{S}), portal (C_{P}), and liver (C_{L}) compartments (see Supplemental Methods, Equations 16-33). Total hepatic flow rate (Q_{L}) was comprised of both hepatic arterial (q_{SL}) and portal venous (q_{PL}) inflows to the liver (Equation 3).

Q_{L}=q_{SL}+q_{PL} (3)

Using the previous estimate for liver weight, w_{L}, the initial estimate for total hepatic flow rate (Q_{L, init}) was approximately 1 L·min⁻¹·kg⁻¹ liver wet weight [17, 23].

Splanchnic circulation (q_{SP}) is defined as the blood flow to the abdominal gastrointestinal organs, including the stomach, liver, spleen, pancreas, and intestines. A variety of mechanisms for splanchnic blood flow control have been proposed in which the splanchnic vascular bed functions with an autoregulatory capacity to maintain a constant blood flow across a range of perfusion pressures [24]. For the purpose of this model, q_{SP} was assumed to be proportional to the total cardiac output. Since splanchnic circulation accounts for approximately 75% of the total liver inflow (with the remaining 25% supplied by the hepatic artery) [17], q_{SP} was defined as a constant flow rate at 75% of the total liver inflow (Q_{L}).

The liver's unique dual blood supply draws from both the hepatic artery and the portal vein. Hepatic arterial inflow (q_{SL}) accounts for about 25% of the total inflow to the liver [17], is highly adaptable [25], and is capable of compensating for changes in portal venous flow with studies suggesting that 25%-60% of decreased portal flow can be buffered by the hepatic artery [26, 27]. Thus, q_{SL} was modeled as an adaptive flow rate with an initial estimate q_{SL, init} of 25% of the total inflow to the liver (Q_{L}).

In health, the portal venous inflow to the liver (q_{PL}) equals the splanchnic arterial flow rate (q_{SP}). However, in the presence of collateral circulation (e.g., portosystemic shunts or esophageal and gastric varices), portal venous inflow is reduced by the shunt flow (q_{PS}) which bypasses the liver (Equation 4).

q_{PL}=q_{SP}−q_{PS} (4)

Lastly, the total hepatic venous return flow to systemic circulation (q_{LS}) is equal to the total inflow to the liver, Q_{L}.

### Hepatic Extraction

Hepatic extraction ratio (ER) is defined as the fraction of drug entering the liver which is irreversibly removed during a single pass through the liver [28, 29]. Drugs with high ER are rapidly cleared by the liver, whereby the clearance depends primarily on hepatic blood flow. The hepatic uptake of bile acids is exceptionally efficient with extraction ratios of 50-90% depending on the bile acid structure [30]. Gilmore and Thompson studied the clearance of cholate in 14 human controls and found mean (standard deviation) extraction ratios of 77.0% (7.5%) [31]. Similarly, O'Maille, Richards, and Short measured an extraction ratio of 79.0% (8.0%) in dogs [32]. For cholate, an extraction ratio greater than 0.7 is considered high [33]; thus, clearance is primarily driven by flow to the liver. Since the concentration of cholate in systemic venous plasma is relatively low, and because it's extensively bound to albumin, the amount of cholate eliminated in urine is negligible [30] and, therefore, it was assumed that the administered cholate was eliminated entirely by the liver.

In the CM, two phases of IV clearance were modelled to account for the initial rapid elimination of 13C-CA followed by a slower clearance phase. The fast phase extraction ratio (ER_{fast}) was defined as the ratio of the total IV clearance (0 to 180 minutes), Cl_{IV}, to the first phase of clearance (0 to 20 minutes), Cl_{FP}, and was approximately 75% for all subjects regardless of liver disease status (Equation 5).

$${ER}_{fast} = \frac{{Cl}_{IV}}{{Cl}_{FP}} = \frac{{C}_{0}}{{k}_{fast} \cdot {AUC}_{IV}} \qquad ( 5 )$$

Here, AUC_{IV} is the area under the IV curve, the parameter k_{fast} is estimated from the slope of log concentration-time IV curves between 5- and 20-minute timepoints [14, 34], and C₀ is the extrapolated initial 13C-CA concentration. Free cholate is eliminated from the liver with total hepatic clearance defined as the hepatic inflow times the extraction ratio [28, 29]. Due to the high extraction ratio of cholate (ER>0.7) and relatively constant intrinsic hepatocyte clearance across the spectrum of liver disease, differences in cholate clearance in CLD are assumed to be primarily attributed to altered flow to the liver. During the first phase of elimination (approximately 0-20 minutes), the measured 13C-CA concentration drops rapidly with clearance Cl_{IV}, primarily due to hepatic arterial flow to the liver (Equation 6).

Cl_{IV}=Q_{L}·ER_{fast} (6)

Following this fast phase of IV clearance (20-180 minutes) and for the entire oral clearance is a slow phase of hepatic clearance (Cl_{H}), in which the effects of anatomic shunt flow and collateral circulation become apparent and differences in liver function are readily distinguished (Equation 7).

Cl_{H}=Q_{L}·ER_{slow} (7)

For this slow phase of clearance, ER_{slow} was determined through parameter estimation with an initial estimate equal to ER_{fast}.

### Cholate Binding

The hepatic uptake of organic anions has been previously described according to the conventional “free-drug” hypothesis in which the concentration of free ligand controls the hepatic uptake rate [35, 36]. However, despite their highly efficient hepatic uptake, organic anions have a strong affinity for binding to serum albumin. Alternative “albumin-mediated” uptake models have been proposed to explain efficient uptake [37, 38]. Here, cholate was modeled according to three states: (1) albumin-bound noncellular intravascular cholate, (2) extravascular cholate distributed to cells/tissues, and (3) free/unbound cholate. All cholate was assumed to be completely bound to albumin at the time of dose administration, and a fraction of the administered dose was assumed to leave the plasma and enter extravascular compartments of the body (i.e., distributed to cells and tissues). An initial estimate for the albumin-bound noncellular intravascular fraction (f_{A, init}) was calculated by the ratio of systemic compartment volume to the volume of distribution (V_{S}/V_{d}), and the final intravascular fraction (f_{A}) was determined through parameter estimation. Albumin-bound cholate dissociated to free/unbound cholate in the liver with rate constant k_{AU}. Similarly, cholate distributed to cells and tissues dissociated to albumin-bound noncellular intravascular cholate in the liver with rate constant k_{BA}. The dissociation rate constants k_{AU} and k_{BA} were estimated using internal HepQuant data involving lean controls, and the average values of k_{AU} (0.16 min⁻¹) and kBA (5.17 ⁻¹) were used for all subjects. Systemic concentrations of cholate were comprised of the albumin-bound noncellular intravascular cholate and free/unbound cholate, and only the free/unbound cholate was eliminated by the liver.

### Dose Administration

Absolute bioavailability (F), or “SHUNT”, compares the bioavailability of orally administered d4-CA with the bioavailability of IV-administered 13C-CA and is calculated by the ratio of areas under the oral and IV curves (AUC_{PO} and AUC_{IV}, respectively) and normalized by the IV dose (D_{IV}) and oral dose (D_{PO}) (Equation 8) [39].

$$F = \frac{{AUC}_{PO} \cdot {D}_{IV}}{{AUC}_{IV} \cdot {D}_{PO}} \qquad ( 8 )$$

IV dose administration was modeled as a single bolus directly into the systemic compartment at time 0. Oral dose administration was modeled via a flexible transit model [40, 41] which has been demonstrated to closely describe absorption delay observed in oral drug administration. A transit model was adapted to describe the passage of D_{PO} through a series of n non-integer hypothetical transit compartments to simulate drug absorption delay and account for first-pass extraction. Equation 9 describes the rate of change of the amount of d4-CA (dA_{d4}/dt) entering systemic circulation.

$$\frac{{dA}_{d 4}}{dt} = {D}_{PO} \cdot {F}_{FP} \cdot {F}_{adj} \cdot {k}_{TR} \cdot \frac{{( {k}_{TR} \cdot t )}^{n} \cdot {e}^{- {k}_{TR} \cdot t}}{\sqrt{2 π} \cdot {n}^{π + 0.5} \cdot {e}^{- n}} \qquad ( 9 )$$

Here, t is time in minutes; km is the transit rate constant between compartments (Equation 10), where MTT is the mean transit time of a d4-CA molecule through intestinal absorption and into systemic circulation; F_{FP} is the estimated first-pass bioavailability (Equation 11) where the fraction of oral dose available equals the shunted fraction plus the unextracted fraction that reached the liver; and F_{adj} is an adjustment factor which scales F_{FP} to equal the absolute bioavailability, F.

$$\begin{matrix}{k}_{TR} = \frac{n + 1}{MTT} & ( 10 ) \\ {F}_{FP} = ( \frac{{q}_{PS}}{{q}_{SP}} + [ 1 - {ER}_{slow} ] \frac{{q}_{LL}}{{q}_{SP}} ) & ( 11 )\end{matrix}$$

### Indices of Hepatic Disease

The following metrics were estimated by the HepQuant SHUNT test and have previously demonstrated associations with liver function.

Portal and systemic hepatic filtration rates (HFR_{P} and HFR_{S}) are clearances adjusted for body weight (Equations 12 and 13).

$$\begin{matrix}{HFR}_{P} = \frac{{D}_{PO}}{{AUC}_{PO} \cdot BW} & ( 12 ) \\ {HFR}_{S} = \frac{{D}_{IV}}{{AUC}_{IV} \cdot BW} & ( 13 )\end{matrix}$$

SHUNT is the absolute bioavailability of orally administered d4-CA (Equation 8).

STAT is a single-point estimate of DSI which uses the d4-CA concentration at 60 minutes normalized to 75 kg body weight.

DSI is a score indicative of overall liver function comprising both portal and systemic HFR. The DSI is intended to convey unique information regarding fibrosis stage and clinical stages of cirrhosis, and modeling these outputs for prediction of clinical outcomes produced the DSI in Equation 14 [14, 42-44]. Here, HFR_{P,max} and HFR_{S,max} are the upper limits of clearance of healthy controls, and A is a factor to scale DSI from 0 to 50.

$$DSI = A \cdot \sqrt{{( ln [ \frac{{HFR}_{P , max}}{{HFR}_{P}} ] )}^{2} + {( ln [ \frac{{HFR}_{S , max}}{{HFR}_{S}} ] )}^{2}} \qquad ( 14 )$$

Hepatic Reserve (HR) is a numerical index representing overall hepatic health with values between 0 and 100 (Equation 15). Here, HR uses HFR_{P} and HFR_{S} indexed to lean controls minus one standard deviation (HFR_{P,lean} and HFR_{S,lean}) with constant A to scale HR from 100 to 0.

$$H R = 100 - A \cdot \sqrt{{( ln [ \frac{{HFR}_{P , lean}}{{HFR}_{P}} ] )}^{2} + {( ln [ \frac{{HFR}_{S , lean}}{{HFR}_{S}} ] )}^{2}} \qquad ( 15 )$$

### Minimal Model

The MM test parameters were calculated according to methods published previously [14]. Briefly, the “minimal model” refers to the noncompartmental mathematical methods which reduced the prior test's timepoints from 14 to five, representing an accurate and lower cost method that minimizes phlebotomy and time commitments by patients and decreases the burden on laboratory analysis. The MINI test parameters calculated in this study were the same indices of hepatic disease as the CM (DSI, HR, HFR_{P}, HFR_{S}, STAT, and SHUNT) but instead used the MINI curve fits to derive these parameters.

### Model Development and Statistical Analysis

The CM parameters were estimated using MATLAB 2020b SimBiology (The MathWorks, Inc., Natick, MA, USA). Individual CMs were fit to subjects' IV and oral profiles. Goodness of fit was assessed with mean square error (MSE), Akaike Information Criterion (AIC), and R². Parameter estimation was performed by nonlinear least-squares regression according to a modified constant error model in which some weights were reduced to reflect higher uncertainty, for example, during the rapid distribution phase of IV clearance (5 min 13C-CA weight of 0.33) and at low concentrations of oral clearance (5 min and 90 min d4-CA weights of 0.67).

The compartmental model ordinary differential equations (ODEs) are listed below and include a total of 18 ODEs as shown in equations (1′) to (18′) for the following: 2 doses of cholate—intravenous 13C-CA (“IV”) and oral d4-CA (“PO”); 3 binding configurations of cholate—albumin bound (“A”), bound/distributed to cells and tissues (“B”), and unbound/free (“U”); and 3 compartments—systemic (“S”), portal (“P”), and liver (“L”). The ODEs are shown as Equations 1′ to 18′.

The cholate concentration measured by the HepQuant cholate SHUNT test is only the albumin-bound and free cholate found in serum; thus, the concentration of extravascular cholate bound/distributed to cells and tissues (“BIV” and “BPO”) does not contribute to the measured concentration when fitting the data.

The disclosure provides a compartmental model (CM) comprising two or more, three or more, four or more, five or more, six or more, seven or more, two to ten, two to eight, three to seven, or three compartments. In some examples, the compartmental model comprises at least three compartments comprising systemic, portal, and liver compartments of different volumes in which drug concentrations are described by the following series of kinetic equations (1′) to (18′) simulating the transfer of flow from one compartment to another. The equations (1′) to (18′) are described using an orally administered first distinguishable cholate (e.g., d4-CA) and an intravenously administered second distinguishable cholate (e.g., 13C-CA), however, any orally administered first and intravenously administered second distinguishable cholates may be employed.

### Systemic Compartment 13C-CA

### Albumin Bound:

$$\frac{{dC}_{S , AIV}}{dt} = \frac{1}{{V}_{S}} ( {f}_{A} \cdot {D}_{IV} - [ {q}_{S P} + {q}_{S L} ] {C}_{S , AIV} + {q}_{P S} \cdot {C}_{P , AIV} + {q}_{LS} \cdot {C}_{L , AIV} ) \qquad ( 1 ’ )$$

### Bound/Distributed to Cells/Tissues:

$$\frac{{dC}_{S , BIV}}{dt} = \frac{1}{{V}_{S}} ( [ 1 - {f}_{A} ] {D}_{IV} - [ {q}_{S P} + {q}_{S L} ] {C}_{S , BIV} + {q}_{P S} \cdot {C}_{P , BIV} + {q}_{LS} \cdot {C}_{L , BIV} ) \qquad ( 2 ’ )$$

### Unbound/Free Cholate:

$$\frac{{dC}_{S , UIV}}{dt} = \frac{1}{{V}_{S}} ( - [ {q}_{S P} + {q}_{S L} ] {C}_{S , UIV} + {q}_{P S} \cdot {C}_{P , UIV} + {q}_{L S} \cdot {C}_{L , UIV} ) \qquad ( 3 ’ )$$

### Systemic Compartment d4-CA

### Albumin Bound:

$$\frac{{dC}_{S , APO}}{dt} = \frac{1}{{V}_{S}} ( - [ {q}_{S P} + {q}_{S L} ] {C}_{S , APO} + {q}_{P S} \cdot {C}_{P , APO} + {q}_{LS} \cdot {C}_{L , APO} ) \qquad ( 4 ’ )$$

### Bound/Distributed to Cells/Tissues:

$$\frac{{dC}_{S , BPO}}{dt} = \frac{1}{{V}_{S}} ( - [ {q}_{S P} + {q}_{S L} ] {C}_{S , BPO} + {q}_{P S} \cdot {C}_{P , BPO} + {q}_{LS} \cdot {C}_{L , BPO} ) \qquad ( 5 ’ )$$

### Unbound/Free Cholate:

$$\frac{{dC}_{S , UPO}}{dt} = \frac{1}{{V}_{S}} ( - [ {q}_{S P} + {q}_{S L} ] {C}_{S , UPO} + {q}_{P S} \cdot {C}_{P , UPO} + {q}_{LS} \cdot {C}_{L , UPO} ) \qquad ( 6 ’ )$$

### Portal Compartment 13C-CA

### Albumin Bound:

$$\frac{{dC}_{P , AIV}}{dt} = \frac{1}{{V}_{P}} ( {q}_{SP} \cdot {C}_{S , AIV} - [ {q}_{P L} + {q}_{PS} ] {C}_{P , AIV} ) \qquad ( 7 ’ )$$

### Bound/Distributed to Cells/Tissues:

$$\frac{{dC}_{P , BIV}}{dt} = \frac{1}{{V}_{P}} ( {q}_{SP} \cdot {C}_{S , BIV} - [ {q}_{P L} + {q}_{PS} ] {C}_{P , BIV} ) \qquad ( 8 ’ )$$

### Unbound/Free Cholate:

$$\frac{{dC}_{P , UIV}}{dt} = \frac{1}{{V}_{P}} ( {q}_{SP} \cdot {C}_{S , UIV} - [ {q}_{P L} + {q}_{PS} ] {C}_{P , UIV} ) \qquad ( 9 ’ )$$

### Portal Compartment d4-CA

### Albumin Bound:

$$\frac{{dC}_{P , APO}}{dt} = \frac{1}{{V}_{P}} ( {f}_{A} \cdot {D}_{P O} + {q}_{S P} \cdot {C}_{S , APO} - [ {q}_{P L} + {q}_{P S} ] {C}_{P , APO} ) \qquad ( 10 ’ )$$

### Bound/Distributed to Cells/Tissues:

$$\frac{{dC}_{P , BPO}}{dt} = \frac{1}{{V}_{P}} ( [ 1 - {f}_{A} ] {D}_{P O} + {q}_{S P} \cdot {C}_{S , BPO} - [ {q}_{P L} + {q}_{P S} ] {C}_{P , BPO} ) \qquad ( 11 ’ )$$

### Unbound/Free Cholate:

$$\frac{{dC}_{P , UPO}}{d t} = \frac{1}{{V}_{P}} ( {q}_{SP} \cdot {C}_{S , UPO} - [ {q}_{P L} + {q}_{P S} ] {C}_{P , UPO} ) \qquad ( 12 ’ )$$

### Liver Compartment 13C-CA

### Albumin Bound:

$$\frac{{dC}_{L , AIV}}{dt} = \frac{1}{{V}_{L}} ( {k}_{B A} \cdot {C}_{L , BIV} \cdot {V}_{L} - [ {k}_{A U} \cdot {V}_{L} + {q}_{L S} ] {C}_{L , AIV} + {q}_{P L} \cdot {C}_{P , AIV} + {q}_{S L} \cdot {C}_{S , AIV} ) \qquad ( 13 ’ )$$

### Bound/Distributed to Cells/Tissues:

$$\frac{{dC}_{L , BIV}}{dt} = \frac{1}{{V}_{L}} ( - [ {k}_{B A} \cdot {V}_{L} + {q}_{L S} ] {C}_{L , BIV} + {q}_{P L} \cdot {C}_{P , BIV} + {q}_{SL} \cdot {C}_{S , BIV} ) \qquad ( 14 ’ )$$

### Unbound/Free Cholate:

$$\frac{{dC}_{L , UIV}}{dt} = \frac{1}{{V}_{L}} ( {k}_{A U} \cdot {C}_{L , AIV} \cdot {V}_{L} - [ C {l}_{IV} + {q}_{L S} ] {C}_{L , UIV} + {q}_{P L} \cdot {C}_{P , UIV} + {q}_{SL} \cdot {C}_{S , UIV} ) \qquad ( 15 ’ )$$

### Liver Compartment d4-CA

### Albumin Bound:

$$\frac{{dC}_{L , APO}}{dt} = \frac{1}{{V}_{L}} ( {k}_{B A} \cdot {C}_{L , B P O} \cdot {V}_{L} - [ {k}_{A U} \cdot {V}_{L} + {q}_{L S} ] {C}_{L , APO} + {q}_{P L} \cdot {C}_{P , APO} + {q}_{S L} \cdot {C}_{S , APO} ) \qquad ( 16 ’ )$$

### Bound/Distributed to Cells/Tissues:

$$\frac{{dC}_{L , BPO}}{dt} = \frac{1}{{V}_{L}} ( - [ {k}_{B A} \cdot {V}_{L} + {q}_{L S} ] {C}_{L , BPO} + {q}_{P L} \cdot {C}_{P , BPO} + {q}_{S L} \cdot {C}_{S , BPO} ) \qquad ( 17 ’ )$$

### Unbound/Free Cholate:

$$\frac{{dC}_{L , UPO}}{dt} = \frac{1}{{V}_{L}} ( {k}_{A U} \cdot {C}_{L , APO} \cdot {V}_{L} - [ C {l}_{H} + {q}_{L S} ] {C}_{L , U P O} + {q}_{P L} \cdot {C}_{P , U P O} + {q}_{S L} \cdot {C}_{S , U P O} ) . \qquad ( 18 ’ )$$

### Compartment Volumes

The systemic compartment volume (V_{S}) was estimated by multiplying total blood volume by fₚₗₐₛₘₐ (Equation 19′).

V_{S}=TBV·fₚₗₐₛₘₐ (19′)

The volume of the portal compartment (V_{P}) was approximated to be 25% of the systemic volume (Equation 20′).

V_{P}=V_{S}·0.25 (20′)

### Flow Parameters

Total hepatic blood flow is approximately 1 L·min⁻¹·kg⁻¹ liver wet weight [1, 2]. Using the previous estimate for liver weight, w_{L}, the initial estimate for total hepatic flow rate (Q_{L, init}) was approximated by Equation 21′.

Q_{L,init}=1·w_{L}·fₚₗₐₛₘₐ (21′)

Splanchnic circulation (q_{SP}) is described as the blood flow to the abdominal gastrointestinal organs, including the stomach, liver, spleen, pancreas, and intestines [3]. Splanchnic circulation accounts for approximately 75% of the total liver inflow while the remaining 25% is supplied by the hepatic artery [2]. A variety of mechanisms for splanchnic blood flow control have been proposed in which the splanchnic vascular bed functions with an autoregulatory capacity to maintain a constant blood flow across a range of perfusion pressures [3]. Therefore, the flow delivered through splanchnic circulation (q_{SP}) was assumed constant and proportional to total cardiac output (Equation 22′).

q_{SP}=0.75·Q_{L,init} (22′)

The liver's unique dual blood supply draws from the both the hepatic artery and the portal vein. Hepatic arterial inflow (q_{SL}) accounts for about 25% of the total inflow to the liver [2] and is capable of compensating for flow changes in response to changes in portal venous flow, with studies suggesting that 25%-60% of decreased portal flow buffered by the hepatic artery [4, 5]. Thus, q_{SL} was modeled as an adaptive flow rate with an initial estimate q_{SL}, unit of 25% of the total inflow to the liver (Q_{L}) (Equation 23′).

q_{SL,init}=0.25·Q_{L} (23′)

### Hepatic Extraction

In the compartmental model, two phases of IV clearance were modelled to account for the initial rapid elimination of 13C-CA followed by a slow phase. For the fast phase, the extraction ratio (ER_{fast}) was defined as the ratio of total IV clearance, Cl_{IV}, (Equation 24′) to first phase clearance, Cl_{FP}, (Equation 25′) and was approximately 75% for all subjects regardless of liver disease status.

$$C {l}_{IV} = \frac{{D}_{13 C}}{A U {C}_{IV}} \qquad ( 24 ’ )$$

$$C {l}_{F P} = {k}_{fast} \cdot {V}_{d} \qquad ( 25 ’ )$$

Here, AUC_{IV} is the area under the IV curve, the parameter k_{fast} is estimated from the slope of log concentration-time IV curves between 5- and 20-minute timepoints (Equation 26′), C₀ is the extrapolated initial 13C-CA concentration (Equation 27′), and V_{d} is the volume of distribution.

$${k}_{fast} = - \frac{ln ( C [ 20 ] ) - ln ( C [ 5 ] )}{t ( 20 ) - t ( 5 )} \qquad ( 26 ’ )$$

$${C}_{0} = - \frac{C ( 5 )}{{e}^{- {k}_{fast} \cdot t ( 5 )}} \qquad ( 27 ’ )$$

### Cholate Binding

An initial estimate for the noncellular intravascular fraction bound to albumin (i.e., the fraction of cholate not distributed to cells/tissues) (f_{A, init}) was calculated by the ratio of systemic compartment volume to volume of distribution (Equation 28′), and the actual intravascular fraction (f_{A}) was determined through parameter estimation.

$${f}_{A , i n i t} = \frac{V s}{{V}_{d}} \qquad ( 28 ’ )$$

### Dose Administration

The FFP was corrected with an adjustment factor F_{adj} (Equation 29′) which scales the first-pass bioavailability to absolute bioavailability, F.

$${F}_{a d j} = \{ \begin{matrix}\frac{F}{{F}_{F P}} & if F < {F}_{FP} \\ 1 & if F \geq {F}_{FP}\end{matrix} \qquad ( 29 ’ )$$

Compartment model results were compared across subject groups (control, NASH, HCV) and across fibrosis stage (F0-F2 and F3-F4) with P values from 2-sided t-test for significance. Reproducibility was assessed through intraclass correlation coefficient (ICC), which was calculated for indices of hepatic disease for CM and MM as well as for key pharmacokinetic parameters for the CM. The ICC was single rater/measurement, two-way mixed effects model for absolute agreement among measurements [45, 46] with one-sided test for the lower acceptable limit (ICC>0.7) previously defined by a reduction in accuracy of <5% [11]. Correlation plots compare CM and MM for key HepQuant SHUNT test parameters with Deming regression fit to assess systematic differences and coefficient of determination (R²) to assess relationship between the two methods. Bland-Altman plots compare both methods for the same parameters for bias and 95% confidence interval (CI) for upper and lower limits of agreement defined as the mean difference±1.96 standard deviations of the differences [48].

### Parameter Scans

Parameter scans were performed by simulating the CM multiple times, each time varying the value of a parameter while holding other parameters constant. Parameter scans were performed using baseline values from a single control subject with no anatomic shunting (q_{PS}=0) and normal extraction ratio (ER_{fast}=0.78). The anatomic shunting parameter (q_{PS}) was varied as a fraction of the total splanchnic flow rate (q_{SP}) at 0, 25, 50, 75, and 100% (i.e., no shunting to fully shunted flow) while ER was held constant. Similarly, ER was varied at 0.8, 0.6, 0.4, 0.2, 0.1, and 0 (i.e., normal ER to complete loss of hepatocyte function) while q_{PS} was held constant at 0 L·min⁻¹.

### Results

### Selected Subject Characteristics

Selected subject characteristics and standard laboratory test results are shown in Tables 2-3 and were published previously [Translational Research, Volume 233, Burton, James R. et al., “The within-individual reproducibility of the disease severity index from the HepQuant SHUNT test of liver function and physiology”, Pages 5-15, (2021)]. Data are displayed as N or mean±SD.

TABLE 2
Selected characteristics of study subjects.

| Characteristics | Controls | NASH | HCV |
|---|---|---|---|
| No. of subjects | 16 | 16 | 16 |
| Age (y) | 32.9 ± 12.0 | 49.8 ± 11.6 | 55.6 ± 6.7 |
| Gender (M:F) | 8:8 | 7:9 | 13:3 |
| BMI (kg/m²) | 23.0 ± 2.2 | 32.5 ± 5.9 | 28.2 ± 4.1 |
| Race/ethnicity (NHW:H:B:U) | 12:2:1:1 | 14:2:0:0 | 14:1:1:0 |
| Fibrosis stage, N subjects | NA | 0:4:4:5:3 | 3:2:3:4:4 |
| (F0:F1:F2:F3:F4)* | | | |
| Fibrosis score* | NA | 2.4 ± 1.1 | 2.3 ± 1.5 |
| MELD score^{#} | NA | 7.0 ± 1.1 | 7.9 ± 2.0 |
| CTP score^{#} | NA | 5.0 ± 0.0 | 5.4 ± 0.7 |

Abbreviations: B, black race; BMI, body mass index; CTP, Child-Turcotte-Pugh; H, Hispanic; HCV, cases with chronic hepatitis C; M:F, male to female ratio; MELD, model for end-stage liver disease; N, number; NA, not applicable—healthy controls did not undergo liver biopsy and none had clinical disease; NASH, cases with nonalcoholic steatohepatitis; NHW, non-Hispanic white; U, undocumented.
*METAVIR in HCV cases; BRUNT/KLEINER in NASH cases; 2 NASH cases had F3 fibrosis by transient elastography.
^{#}MELD and CTP scores were calculated for the F3/F4 subgroup only.
Endoscopic evaluation was not required for enrollment, but history of variceal hemorrhage was an exclusion.

TABLE 3
Standard laboratory tests for controls and cases of NASH and HCV.
Data are displayed as mean ± SD

| Laboratory tests | Controls | NASH | HCV |
|---|---|---|---|
| Bilirubin (mg/dL) | 0.7 ± 0.3 | 0.8 ± 0.4 | 1.1 ± 0.4** |
| Albumin (g/dL) | 4.0 ± 0.3 | 4.0 ± 0.3 | 3.9 ± 0.4 |
| INR | 1.0 ± 0.1 | 1.0 ± 0.1 | 1.1 ± 0.1 |
| Creatinine (mg/dL) | 1.0 ± 0.1 | 0.8 ± 0.3** | 0.9 ± 0.1 |
| AST (IU/mL) | 22.3 ± 4.8 | 34.0 ± 17.7* | 75.7 ± 64.0** |
| ALT (IU/mL) | 20.7 ± 7.3 | 43.7 ± 33.4* | 100.9 ± 91.4** |
| GGT (IU/mL) | 18.0 ± 6.9 | 44.4 ± 28.4** | 75.8 ± 83.7** |
| Alkaline | 52 ± 17 | 65 ± 24 | 69 ± 30* |
| phosphatase (IU/mL) | | | |
| Platelet count (nL⁻¹) | 243 ± 47 | 193 ± 72* | 152 ± 68*** |
| Hemoglobin (g/dL) | 14.3 ± 1.4 | 14.6 ± 1.7 | 15.1 ± 1.9 |
| WBC (nL⁻¹) | 5.3 ± 1.3 | 6.0 ± 1.8 | 5.9 ± 2.7 |
| Glucose (g/dL) | 84.1 ± 7.0 | 106.3 ± 33.1* | 101.2 ± 16.0*** |
| TSH | 1.6 ± 0.5 | 2.1 ± 1.2 | 2.4 ± 2.2 |
| Cholesterol, total | 162 ± 30 | 167 ± 45 | 143 ± 23 |
| Triglyceride | 79 ± 41 | 168 ± 86** | 105 ± 51 |
| Ferritin | 65 ± 62 | 126 ± 117 | 332 ± 279*** |

Abbreviations: ALT, alanine aminotransferase; AST, aspartate aminotransferase; GGT, gamma-glutamyl transferase; HCV, cases with chronic hepatitis C; INR, international normalized ratio for prothrombin time; N, number; NASH, cases with nonalcoholic steatohepatitis; TSH, thyroid stimulating hormone; WBC, white blood cell count.
*P < 0.05.
**P < 0.01.
***P < 0.001 by 2-sided t-tests, compared to controls.

Controls were 32.9±12.0 years of age, had balanced gender ratio of 8:8 (M:F), and had normal BMI (23.0±2.2). NASH subjects were 49.8±11.6 years of age, had gender ratio of 7:9 (M:F), and had high BMI (32.5±5.9). HCV subjects were 55.6±6.7 years of age, were more likely to be male (13:3, M:F), and had intermediate BMI (28.2±4.1). The majority ( 40/48) of subjects were non-Hispanic white. Compared to controls, NASH and HCV subjects had significantly higher AST, ALT, GGT, and glucose as well as lower platelet count. NASH and HCV groups were classified as either F0-F2 (early) or F3-F4 (advanced) fibrosis stage. Both groups had 8 subjects with F0-F2 and 8 subjects with F3-F4.

### Compartmental Analysis

Compartmental analysis was completed for 16 controls, 16 NASH, and 16 HCV subjects. Inputs to the CM were the 13C-CA and d4-CA concentration measurements, sampling timepoints, oral and IV doses, hematocrit (Hct), and BW. In the CM, transfer between compartments was described by a system of first-order ordinary differential equations along with assumptions from either measured or literature-derived values (Table 4).

TABLE 4
Summary of compartmental model (CM) input parameters and assumptions

| Parameter | Description | Units |
|---|---|---|
| TBV | Total blood volume: TBV = (0.07/sqrt [BMI/22]) · BW | L |
| fₚₗₐₛₘₐ | Plasma fraction: fₚₗₐₛₘₐ = (1 − Hct) | — |
| d_{L} | Liver tissue density: d_{L} = 1.06 g · mL⁻¹ | g · mL⁻¹ |
| w_{L} | Total liver weight (blood weight plus parenchymal weight): w_{L} = 22.46 · BW · d_{L} | g |
| V_{L} | Volume of liver compartment: V_{L} = 27.5 mL per 100 g liver weight times fₚₗₐₛₘₐ | L |
| V_{S} | Volume of systemic compartment: V_{S} = TBV · fₚₗₐₛₘₐ | L |
| V_{P} | Volume of portal compartment: Vp = 0.25 · Vs | L |
| K_{fast} | Hepatic elimination of 13C-CA, initial phase—slope of log IV concentration-time | min⁻¹ |
| | (5-20 min): K_{fast} = −(ln[C₂₀] − ln[C₅])/(t₂₀ − t₅) | |
| C₀ | Extrapolated initial 13C-CA concentration: C₀ = C₅/exp(−k_{fast}_{^{−}}t₅) | μmol · L⁻¹ |
| V_{d} | Volume of distribution: Vd = D_{IV}/C₀ | L |
| f_{A,init} | Albumin-bound noncellular intravascular fraction of total dose estimate: f_{A,init} = | — |
| | V_{S}/V_{d} | |
| k_{AU} | Rate of dissociation of albumin-bound cholate to free cholate (derived from | min⁻¹ |
| | HepQuant internal data): k_{AU} = 0.16 min⁻¹ | |
| k_{BA} | Rate of redistribution from cells/tissues to albumin bound cholate (derived from | min⁻¹ |
| | HepQuant internal data): k_{BA} = 5.17 min⁻¹ | |
| ER_{fast} | Extraction ratio of distribution phase (total IV clearance to first phase clearance): | — |
| | ER_{fast} = C₀/(K_{fast} · AUC_{IV}) | |
| Q_{L,init} | Initial estimate of total hepatic inflow: Q_{L,init} = 1 L · min⁻¹ · kg⁻¹ liver wet weight | L · min⁻¹ · kg⁻¹ |
| | times fₚₗₐₛₘₐ | |
| Q_{L} | Total hepatic inflow: Q_{L} = q_{SL} + q_{PL} | L · min⁻¹ |
| Cl_{IV} | Fast phase clearance of 13C-CA: Cl_{IV} = Q_{L} · ER_{fast} | L · min⁻¹ |
| Cl_{H} | Hepatic clearance of d4-CA and slow phase clearance of 13C-CA: Cl_{H} = Q_{L} · | L · min⁻¹ |
| | ER_{slow} | |
| q_{SP} | Splanchnic arterial circulation: q_{SP} = 0.75 · Q_{L,init} | L · min⁻¹ |
| q_{PL} | Portal inflow rate to liver: q_{PL} = q_{SP} − q_{PS} | L · min⁻¹ |
| q_{LS} | Hepatic return flow rate to systemic circulation: q_{Ls} = q_{PL} + q_{SL} | L · min⁻¹ |
| kₜᵣ | Transit rate: kₜᵣ = (n + 1)/MTT | min⁻¹ |

Model parameters estimated by nonlinear least squares regression are listed in Table 5.

TABLE 5
CM parameters estimated by nonlinear least-squares regression

| Parameter | Description | Initial Value | Bounds | Units |
|---|---|---|---|---|
| q_{SL} | Hepatic arterial circulation | 0.25 · Q_{L,init} | 10%-90% of Q_{L,init} | L · min⁻¹ |
| q_{PS} | Anatomic shunt flow | 0 | 0-q_{SP} | L · min⁻¹ |
| MTT | Mean transit time | Time at oral curve | ±50% | min |
| | | maximum | | |
| | | concentration | | |
| n | Number of transit compartments | 3 | 1-5 | — |
| J_{A} | Albumin-bound noncellular | f_{A,init} | ±50% | — |
| | intravascular fraction of total dose | | | |
| ER_{slow} | Extraction ratio—slow phase and | ER_{fast} | 0.1-1 | — |
| | oral curve | | | |

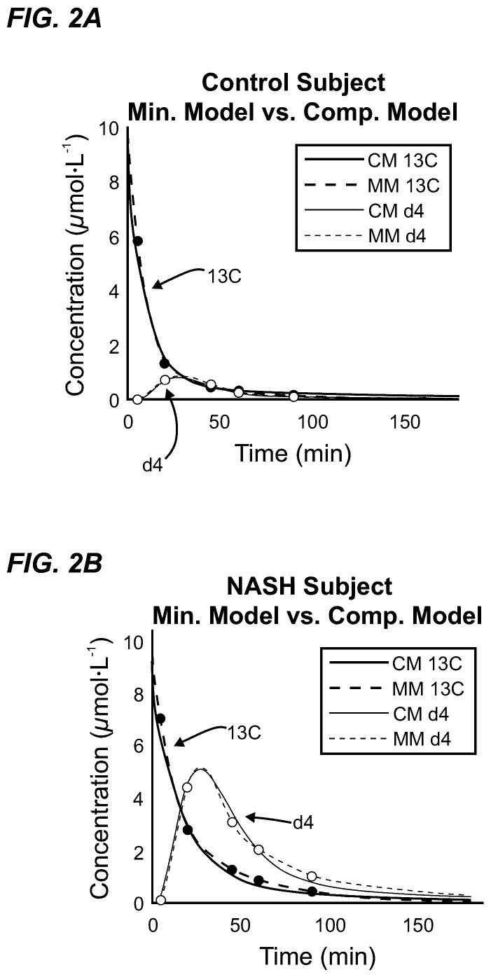

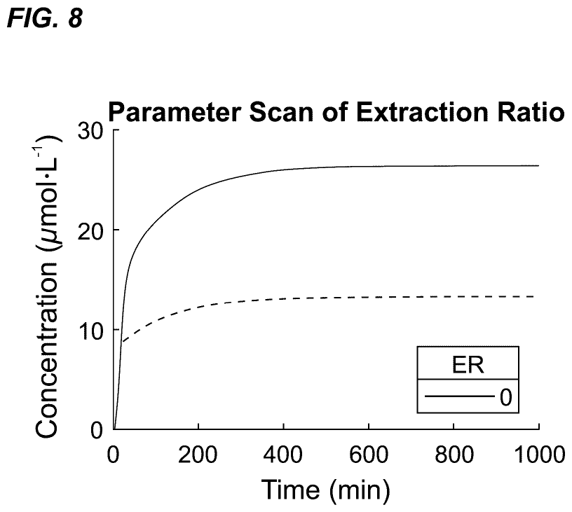

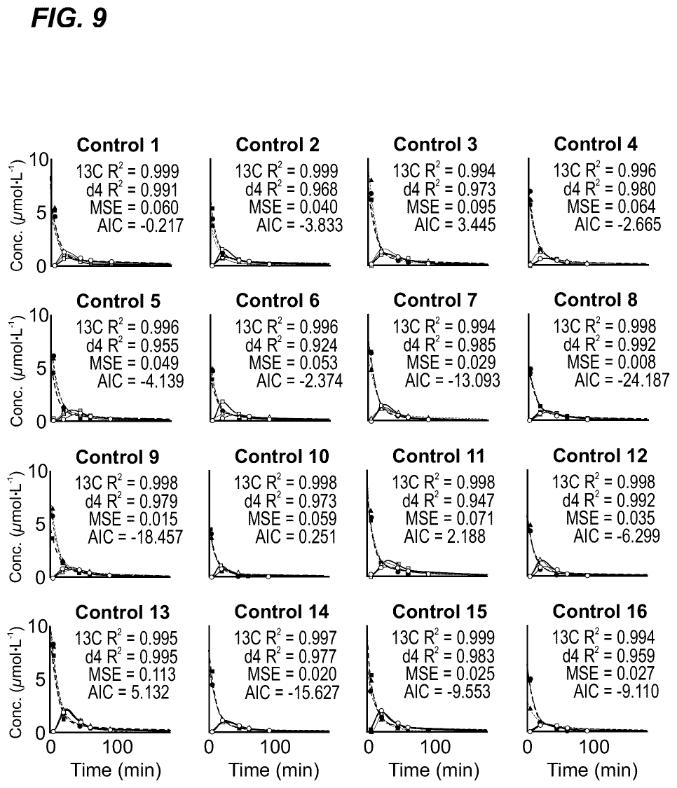

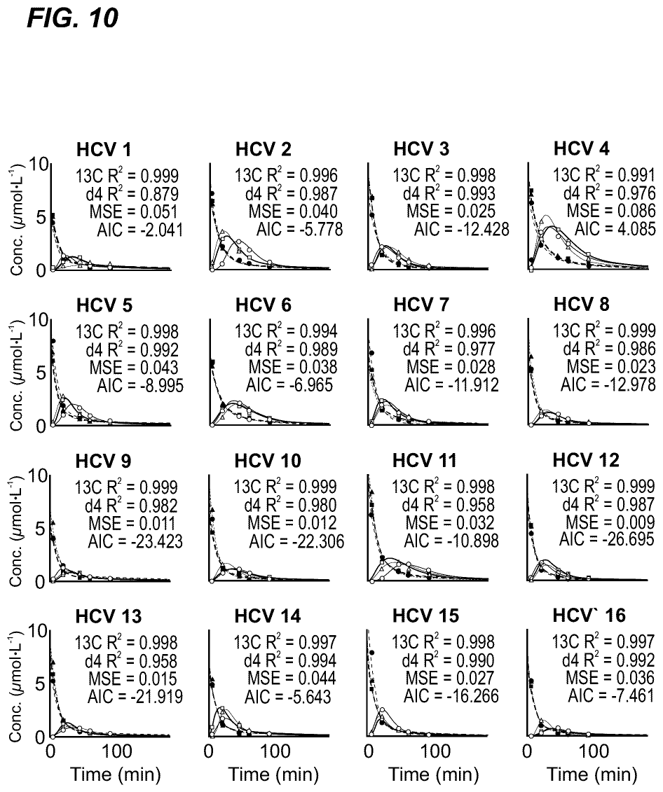

CM simulations and goodness of fit for all 48 subjects (16 controls, 16HCV subjects, 16 NASH subjects) and each of the 3 runs is shown in FIGS. 9, 10, and 11, respectively. The CM fit individual subjects' data with MSE<0.113 for controls, MSE<0.086 for HCV, and <0.077 for NASH subjects. Similarly, adjusted R² values for 13C-CA curves ranged 0.994-0.999 for controls, 0.991-0.999 for HCV, and 0.995-1.000 for NASH. For d4-CA, adjusted R² ranged from 0.924-0.995 for controls, 0.879-0.994 for HCV, and 0.851-0.997 for NASH. FIGS. 2A and 2B shows an example of the CM simulations alongside the MM results for a control subject (FIG. 2A) and a NASH subject (FIG. 2B), showing significant overlap between the CM and MINI methods. The CM parameters were compared across subject groups (controls, NASH, and HCV) (Table 6).

TABLE 6
CM parameters (mean ± SD) by disease category

| | | | | P value | | |
|---|---|---|---|---|---|---|
| | Control | NASH | HCV | Control vs | Control vs | NASH vs |
| | N = 16 | N = 16 | N = 16 | NASH | HCV | HCV |
| DSI | 9.62 ± 3.28 | 16.10 ± 3.61 | 18.24 ± 5.90 | <0.001 | <0.001 | 0.2251 |
| HR | 98.58 ± 2.42 | 88.49 ± 7.31 | 83.74 ± 13.19 | <0.001 | <0.001 | 0.2171 |
| HFR_{S} | 6.09 ± 0.95 | 4.65 ± 0.80 | 4.44 ± 1.08 | <0.001 | <0.001 | 0.5304 |
| (mL · kg⁻¹ · min⁻¹) | | | | | | |
| HFR_{P} | 26.99 ± 8.27 | 15.87 ± 5.41 | 13.76 ± 6.20 | <0.001 | <0.001 | 0.3138 |
| (mL · kg⁻¹ · min⁻¹) | | | | | | |
| SHUNT | 0.24 ± 0.05 | 0.32 ± 0.07 | 0.37 ± 0.13 | <0.01 | <0.001 | 0.1524 |
| STAT (μmol · L⁻¹)* | 0.36 ± 0.10 | 0.64 ± 0.21 | 0.92 ± 0.65 | <0.001 | <0.01 | 0.1106 |
| q_{PS} (L · min⁻¹)* | 0.02 ± 0.04 | 0.11 ± 0.09 | 0.13 ± 0.14 | <0.01 | <0.01 | 0.5277 |
| q_{PL} (L · min⁻¹)* | 0.75 ± 0.07 | 0.66 ± 0.11 | 0.62 ± 0.15 | <0.01 | <0.01 | 0.3972 |
| q_{SP} (L · min⁻¹)* | 0.77 ± 0.05 | 0.76 ± 0.05 | 0.75 ± 0.07 | 0.6118 | 0.2958 | 0.5514 |
| q_{LS} (L · min⁻¹)* | 1.36 ± 0.21 | 1.07 ± 0.20 | 1.03 ± 0.25 | <0.001 | <0.001 | 0.6434 |
| q_{SL} (L · min⁻¹)* | 0.61 ± 0.17 | 0.41 ± 0.11 | 0.41 ± 0.13 | <0.001 | <0.001 | 0.9608 |
| ER_{slow} | 0.66 ± 0.11 | 0.56 ± 0.08 | 0.51 ± 0.08 | <0.01 | <0.001 | 0.1193 |
| ER_{fast} | 0.78 ± 0.02 | 0.78 ± 0.02 | 0.74 ± 0.03 | 0.5473 | <0.001 | <0.001 |
| V_{d} (L · kg⁻¹) | 0.09 ± 0.02 | 0.06 ± 0.01 | 0.07 ± 0.01 | <0.001 | <0.001 | 0.2968 |
| Cl_{H} (L · min⁻¹)* | 0.91 ± 0.24 | 0.60 ± 0.16 | 0.54 ± 0.20 | <0.001 | <0.001 | 0.3350 |
| MTT (min) | 21.11 ± 3.65 | 25.58 ± 9.35 | 24.26 ± 6.64 | 0.0853 | 0.1078 | 0.6475 |

Abbreviations:
DSI, disease severity index;
HR, hepatic reserve;
HFR_{S}, systemic hepatic filtration rate;
HFR_{P}, portal hepatic filtration rate;
q_{SP}, splanchnic arterial flow rate;
q_{PS}, anatomic shunt flow rate;
q_{PL}, portal venous flow rate;
q_{LS}, hepatic return to systemic circulation flow rate;
q_{SL}, hepatic arterial flow rate;
Cl_{H}, hepatic clearance;
V_{d}, volume of distribution;
MTT, mean transit time.
P values from 2-sided t-tests.
The average of triplicate tests for each individual subject was used to generate the values for table 3.
*Normalized for body weight

As shown in Table 6, compared to the controls, NASH and HCV subjects had significantly higher DSI, SHUNT, STAT, and q_{PS}, and lower HR, HFR_{S}, HFR_{P}, q_{PL}, q_{LS}, q_{SL}, ER_{slow}, and Cl_{H}. The extraction ratio (ER_{fast}) was lower in HCV subjects relative to control and NASH subjects. These results suggest that relative to the controls, NASH and HCV subjects had reduced hepatic arterial and portal venous inflows to the liver, as well as the potential presence of portal-systemic shunting/collaterals and hepatocyte impairment.

The CM parameters were also compared across fibrosis stage (F0-F2 vs F3-F4) (Table 7).

TABLE 7
CM parameters (mean ± SD) by fibrosis stage

| | F0-F2 | F3-F4 | |
|---|---|---|---|
| | N = 16 | N = 16 | P Value |
| DSI | 15.31 ± 3.67 | 19.03 ± 5.43 | <0.05 |
| HR | 90.24 ± 7.52 | 81.99 ± 12.11 | <0.05 |
| HFR_{S} (mL · kg⁻¹ · min⁻¹) | 4.82 ± 0.98 | 4.27 ± 0.85 | 0.1027 |
| HFR_{P} (mL · kg⁻¹ · min⁻¹) | 16.88 ± 5.24 | 12.74 ± 5.79 | <0.05 |
| SHUNT | 0.30 ± 0.08 | 0.38 ± 0.12 | <0.05 |
| STAT (μmol · L⁻¹)* | 0.64 ± 0.21 | 0.92 ± 0.65 | 0.1141 |
| q_{PS} (L · min⁻¹)* | 0.09 ± 0.09 | 0.15 ± 0.14 | 0.1841 |
| q_{PL} (L · min⁻¹)* | 0.66 ± 0.10 | 0.62 ± 0.15 | 0.3589 |
| q_{SP} (L · min⁻¹)* | 0.75 ± 0.06 | 0.76 ± 0.06 | 0.5619 |
| q_{LS} (L · min⁻¹)* | 1.09 ± 0.21 | 1.01 ± 0.23 | 0.3553 |
| q_{SL} (L · min⁻¹)* | 0.43 ± 0.14 | 0.39 ± 0.10 | 0.466 |
| ER_{slow} | 0.58 ± 0.09 | 0.50 ± 0.06 | <0.01 |
| ER_{fast} | 0.77 ± 0.03 | 0.75 ± 0.03 | 0.1959 |
| V_{d}(L · kg⁻¹) | 0.07 ± 0.01 | 0.06 ± 0.01 | <0.05 |
| Cl_{H} (L · min⁻¹)* | 0.64 ± 0.19 | 0.51 ± 0.15 | <0.05 |
| MTT (min) | 25.35 ± 6.52 | 24.49 ± 9.46 | 0.7672 |

Abbreviations: DSI, disease severity index; HR, hepatic reserve; HFR_{S}, systemic hepatic filtration rate; HFR_{P}, portal hepatic filtration rate; q_{SP}, splanchnic arterial flow rate; q_{PS}, anatomic shunt flow rate; q_{PL}, portal venous flow rate; q_{LS}, hepatic return to systemic circulation flow rate; q_{SL}, hepatic arterial flow rate; Cl_{H}, hepatic clearance; V_{d}, volume of distribution; MTT, mean transit time.
P values from 2-sided t-tests.
The average of triplicate tests for each individual subject was used to generate the values for this table.
NASH fibrosis was staged using criteria of BRUNT and KLEINER.
HCV fibrosis was staged according to METAVIR.
*Normalized for body weight

As shown in Table 7, compared to subjects with early (F0-F2) stages of fibrosis, subjects with advanced (F3-F4) stages of fibrosis had higher DSI and SHUNT and lower HR, HFR_{P}, ER_{slow}, and Cl_{H}. Although there were no significant differences detected in the CM' s flow rate parameters (q_{PS}, q_{PL}, q_{SP}, q_{LS}, and q_{SL}) across fibrosis stages, the reduction in extraction ratio (ER_{slow}) points to impairment of hepatocyte function in advanced fibrosis stages relative to early fibrosis stages.

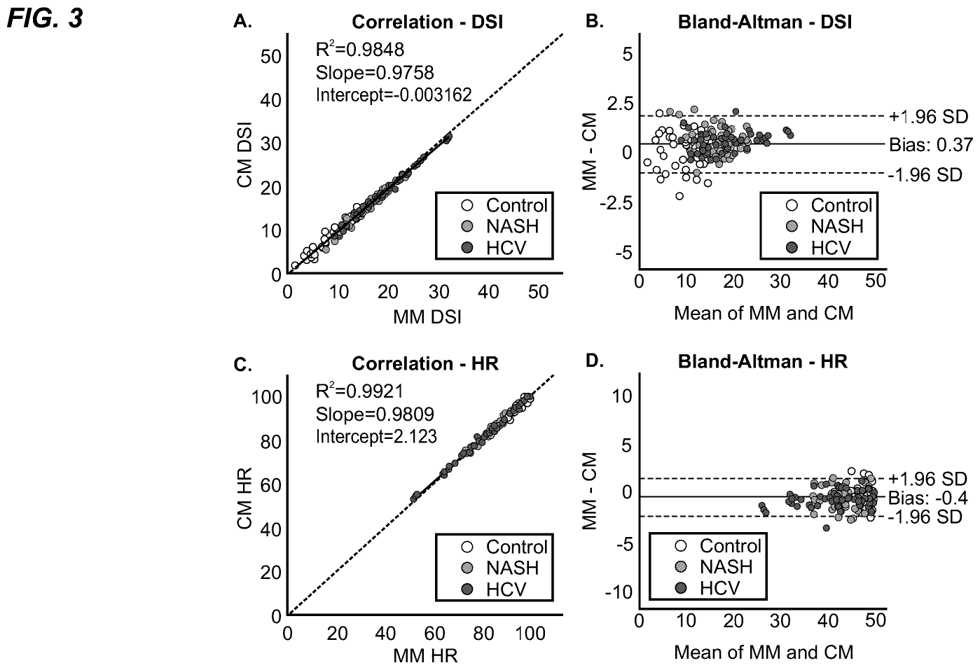

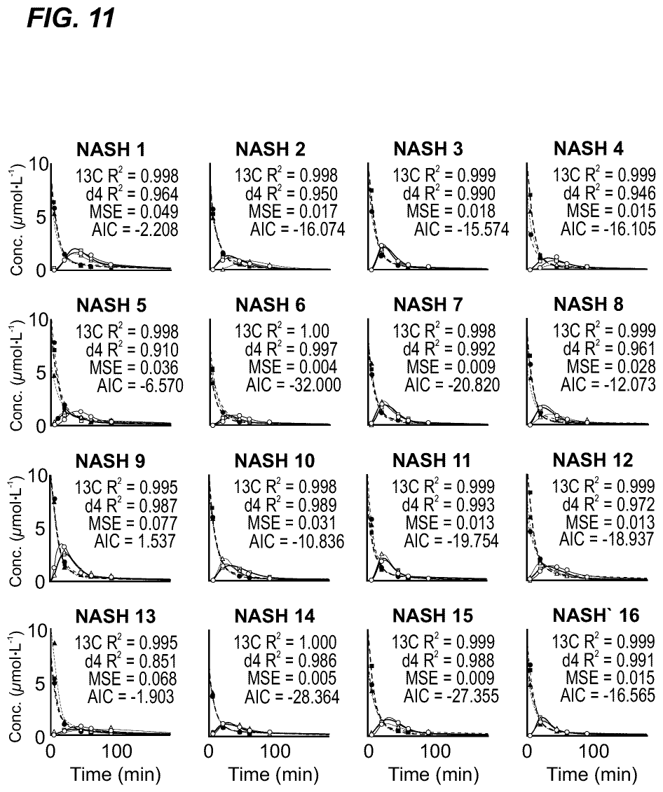

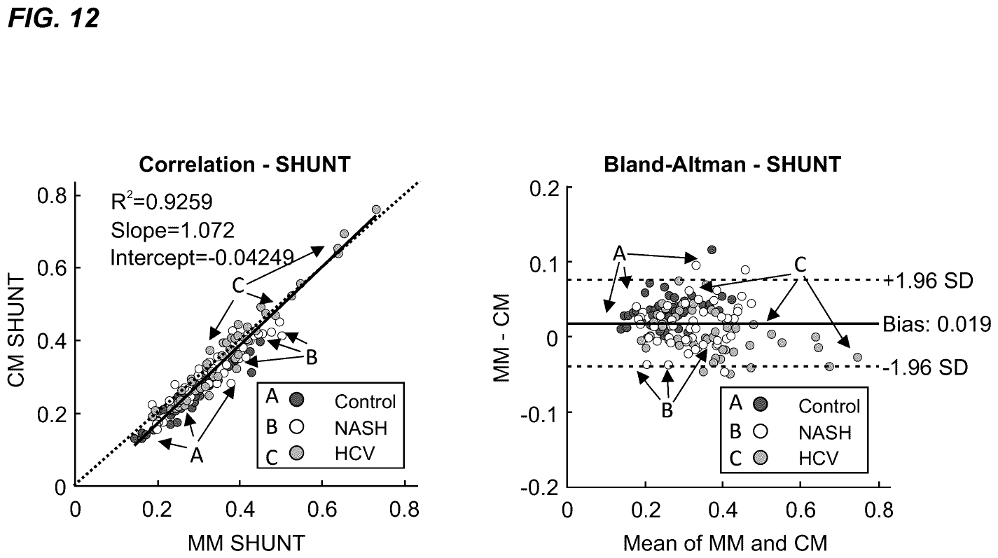

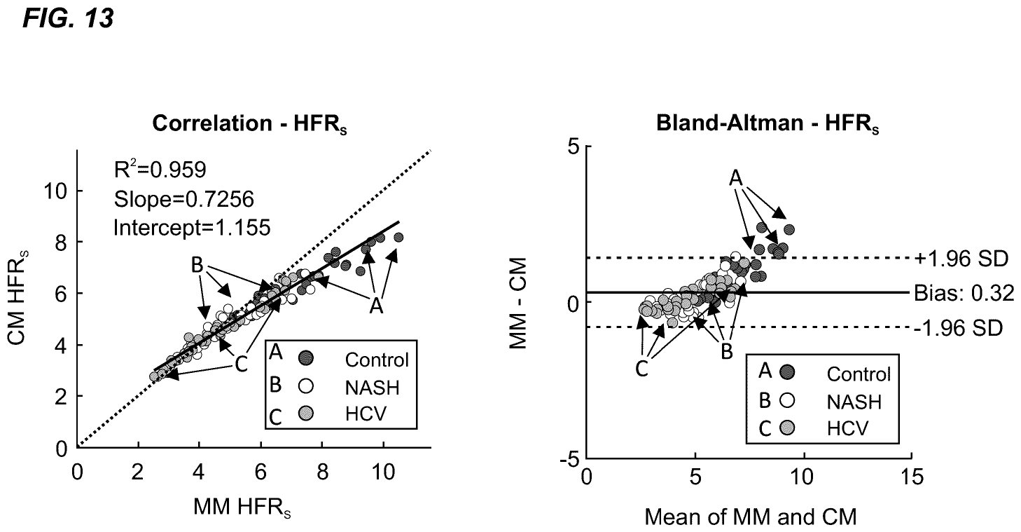

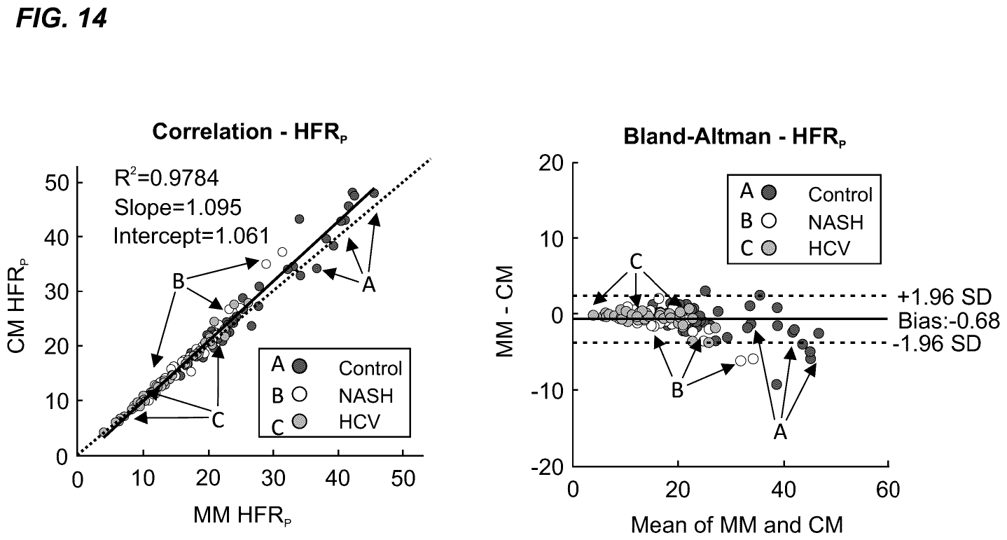

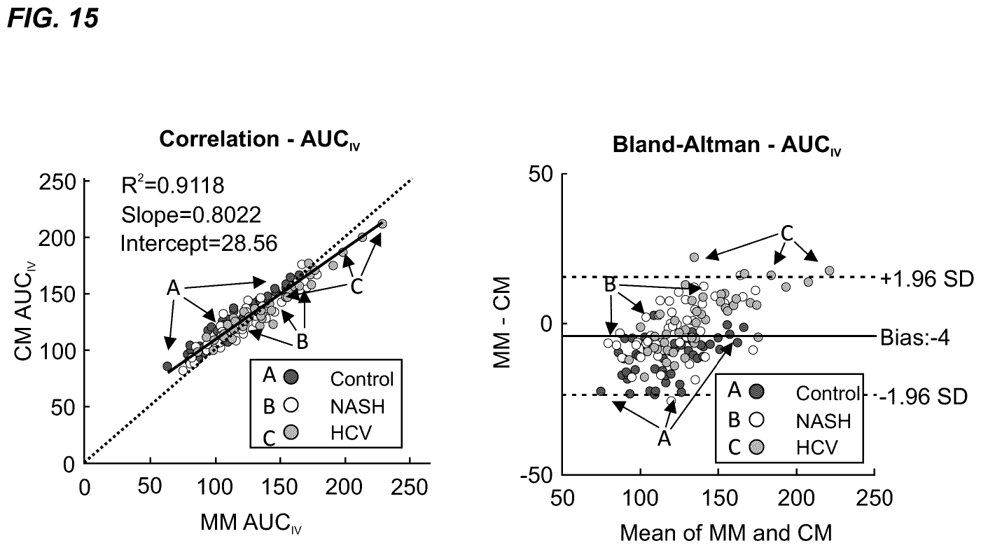

The MM and CM were compared via Deming regression and Bland-Altman analysis (FIG. 3). The DSI derived from the CM correlated with the MINI (R² of 0.9848 [95% CI: 0.9790−0.9891], P<0.001). Bland-Altman analysis showed that the MINI DSI was slightly higher than CM DSI with bias (95% limits of agreement) of 0.3667 (−1.0799−1.8132). Similarly, the CM correlated with the MM in terms of HR, with R² of 0.9921 (95% CI: 0.9890−0.9943), P<0.001. HR calculated by the MINI was lower than the CM, with bias (95% limits of agreement) of −0.4039 (−2.3351−1.5272). FIGS. 12 to 16 show Deming regression and Bland-Altman analysis for SHUNT, HFR_{S}, HFR_{P}, AUC_{IV}, and AUC_{PO}, respectively, with R² values ranging from 0.9118 to 0.9913.

### Reproducibility

The reproducibility was assessed for key indices of hepatic disease from the HepQuant SHUNT test via ICC for MM and CM methods (Table 8).

TABLE 8
Analysis of test reliability for indices of hepatic disease
including all 48 subjects (controls, HCV, NASH).
Intraclass correlation coefficients (ICC) for MM and CM.

| | Minimal Model (MM) | | | Compartmental Model (CM) | | |
|---|---|---|---|---|---|---|
| Index | ICC | 95% CI | P value | ICC | 95% CI | P value |
| DSI | 0.94 | (0.90-0.96) | <0.001 | 0.93 | (0.88-0.95) | <0.001 |
| HR | 0.97 | (0.94-0.98) | <0.001 | 0.96 | (0.94-0.98) | <0.001 |
| HFR_{S} | 0.82 | (0.73-0.89) | 0.008 | 0.88 | (0.82-0.93) | <0.001 |
| HFR_{P} | 0.84 | (0.75-0.90) | 0.0017 | 0.80 | (0.70-0.87) | 0.0288 |
| SHUNT | 0.73 | (0.60-0.83) | 0.3095 | 0.84 | (0.76-0.90) | 0.0012 |
| STAT | 0.90 | (0.84-0.94) | <0.001 | 0.90 | (0.84-0.94) | <0.001 |

Abbreviations:
DSI, disease severity index;
HR, hepatic reserve;
HFR_{S}, systemic hepatic filtration rate;
HFR_{P}, portal hepatic filtration rate;
ICC, intraclass correlation coefficient.

As shown in Table 6, the ICCs for DSI, HR, HFR_{S}, HFR_{P}, SHUNT, and STAT ranged between 0.80 to 0.96 for CM and 0.73 to 0.97 for MM. Acceptable reproducibility (ICC>0.7) was observed for 100% ( 6/6) and 83% (⅚) of hepatic disease indices for CM and MM, respectively. SHUNT, a measure of the absolute bioavailability of oral d4-CA, had ICC of 0.73 (0.60-0.83, P=0.3095) for MM and 0.84 (0.76-0.90, P=0.0012) for CM.

Reproducibility of the CM-specific parameters were also assessed (Table 9), with ICCs ranging between 0.15 to 1.

TABLE 9
Reproducibility of CM parameters assessed by ICC including all
48 subjects (controls, HCV, NASH)

| CM | | | |
|---|---|---|---|
| Parameter | ICC | 95% CI | P value |
| q_{SP} | 1.00 | (0.99-1.00) | <0.001 |
| q_{PS} | 0.88 | (0.81-0.93) | <0.001 |
| q_{PL} | 0.87 | (0.80-0.92) | <0.001 |
| q_{LS} | 0.85 | (0.78-0.91) | <0.001 |
| q_{SL} | 0.72 | (0.59-0.82) | 0.3819 |
| Cl_{H} | 0.66 | (0.52-0.78) | 0.722 |
| ER_{fast} | 0.48 | (0.30-0.64) | 0.9976 |
| ER_{slow} | 0.43 | (0.25-0.60) | 0.9996 |
| MTT | 0.35 | (0.17-0.53) | 1.0000 |
| V_{d} | 0.15 | (−0.02-0.35) | 1.0000 |

Abbreviations: q_{SP}, splanchnic arterial flow rate; q_{PS}, anatomic shunt flow rate; q_{PL}, portal venous flow rate; q_{LS}, hepatic return to systemic circulation flow rate; q_{SL}, hepatic arterial flow rate; Cl_{H}, hepatic clearance (slow phase and oral clearances); ER_{fast}, extraction ratio, fast phase; ER_{slow}, extraction ratio, slow phase; MTT, mean transit time; V_{d}, volume of distribution.

The flow rate parameters q_{PS}, q_{PL}, q_{SP}, and q_{LS} had acceptable reproducibility (ICC>0.7, P<0.001) while parameters ER_{slow}, ER_{fast}, V_{d}, Cl_{H}, q_{SL}, and MTT failed to meet the acceptability criteria for reproducibility.

### Parameter Scans

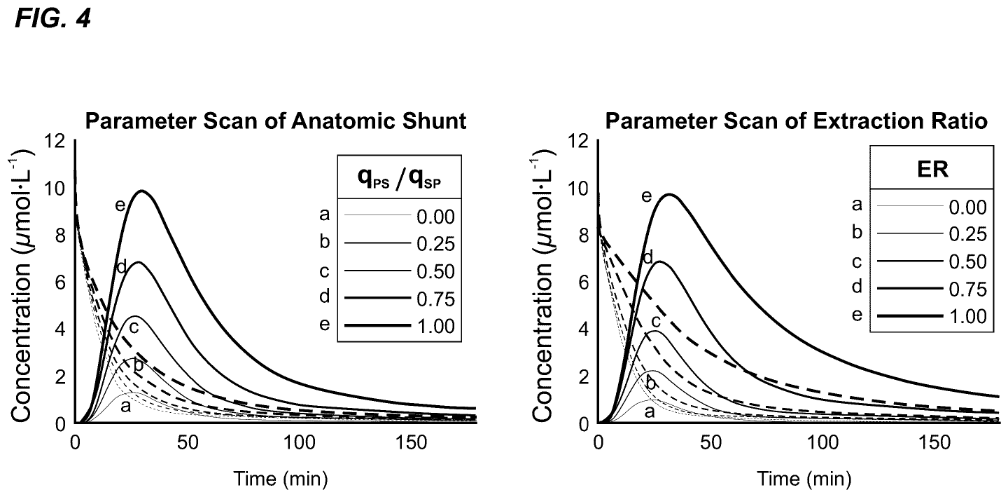

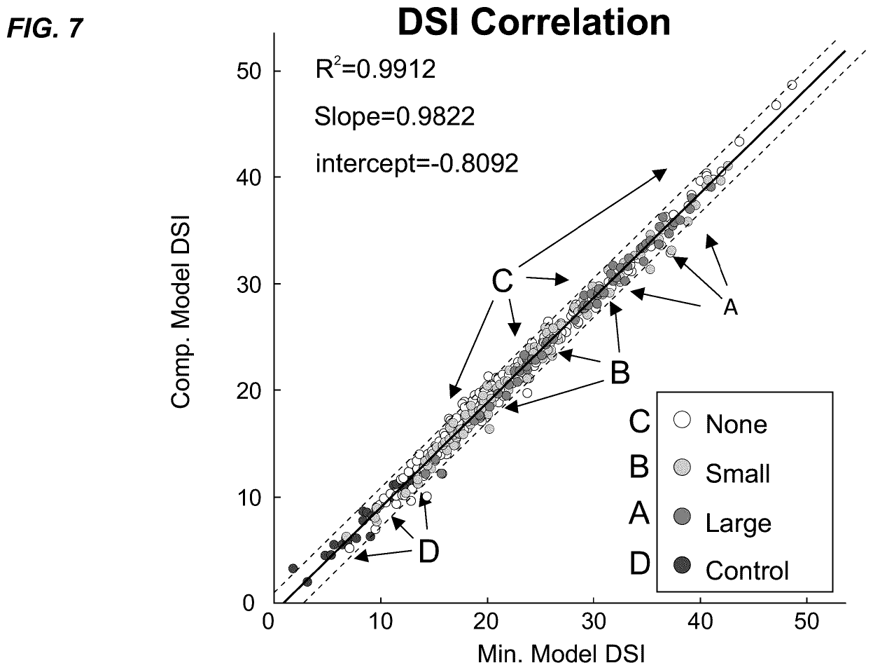

Parameter scans of anatomic shunt (q_{PS}) and extraction ratio (ER) were performed (FIG. 4, left and right panels, respectively) to show the CM response to varying levels of these parameters. When varying the anatomic shunt, the value of q_{PS} was increased from 0 to 100% of the splanchnic arterial flow rate (q_{SP}). As anatomic shunt increased, the apparent spillover of d4-CA rapidly increased, with modest increases in 13C-CA concentration over time. In this example, a normal ER value of 0.78 resulted in relatively efficient extraction of both 13C-CA and d4-CA regardless of the extent of shunting. Likewise, varying the ER between 0.8 and 0.1 resulted in a rapid yet more prolonged increase in d4-CA concentrations with more pronounced and sustained increases in 13C-CA concentrations. As expected, reducing ER to 0 resulted in a d4-CA to 13C-CA concentration ratio of 2:1, mirroring the ratio of the administered oral to IV dose (FIG. 8).

The reproducibility and reliability of the new physiologically based compartmental model (CM) versus the MM was determined. For example, data were analyzed from 16 control, 16 non-alcoholic steatohepatitis (NASH), and 16 hepatitis C virus (HCV) subjects, each with 3 replicate tests conducted on 3 separate days. The CM was compared to the MM for 6 key indices of hepatic disease in terms of intraclass correlation coefficient (ICC) with a lower acceptable limit of 0.7. The CM correlated well with the MM for disease severity index (DSI) with R² (95% confidence interval) of 0.96 (0.94-0.98) (P<0.001). Acceptable reproducibility (ICC>0.7) was observed for 6/6 and ⅚ hepatic disease indices for CM and MM, respectively. SHUNT, a measure of the absolute bioavailability, had ICC of 0.73 (0.60-0.83, P=0.3095) for MM and 0.84 (0.76-0.90, P=0.0012) for CM.

The CM, but not the MM, allowed determination of anatomic shunt and hepatic extraction, and improved reproducibility by reducing the intra-individual/biological variability. The CM may enhance the diagnostic performance of the HepQuant SHUNT test.

The CM demonstrated excellent fits to individual subjects' oral and IV clearance data (all IV curves R²>0.991 and all oral curves R²>0.851) as well as agreement with the MM in terms of correlation and bias for DSI and HR. For both indices, the regression line nearly overlaps identity (slopes of 0.98) with intercepts near the origin. Both the DSI and HR exhibit heteroscedasticity, with higher variability at low DSI/high HR values, due in part to the increased uncertainty of the LC/MS measurements at low d4-CA concentrations. Regardless, the CM provided excellent fits to clearance data from individual subjects and correlated with the previously validated MM.

In terms of reproducibility, the CM had ICC values above the threshold for acceptable reproducibility (ICC>0.7) for all indices of hepatic disease. The MM and CM had ICCs of 0.94 and 0.93, respectively, suggesting that DSI is a highly reproducible measurement and that a single measurement of DSI is reliable regardless of the analysis method. While the MM failed to meet the acceptance criteria for SHUNT with an ICC of 0.73 (95% CI: 0.60-0.83), the CM-calculated SHUNT demonstrated acceptable reproducibility with 0.84 (95% CI: 0.76-0.90). SHUNT is a critical parameter of the HepQuant test which defines the absolute bioavailability of orally administered d4-CA and is associated with portal-systemic shunting. These results suggest that the CM reduced the intra-individual variability in its calculation of SHUNT, due in part to the various physiological parameters included in the CM which influence the response and are consistent across measurements within the same subject. Additionally, SHUNT is a function of the oral and IV AUC, and the CM's simultaneous fit to oral and IV curves, subject to shared pharmacokinetic parameters and physiological constraints, could explain its improved reproducibility.

Some parameters specific to the CM (ER, V_{d}, Cl_{H}, q_{LS}, and MTT) failed to meet the acceptance criteria of ICC<0.7. The ER, V_{d}, and Cl_{H} are all highly dependent on the test administration, and slight variations in the timing of serum samplings has a large effect on these parameters, especially at 5- and 20-minute samples. Hepatic arterial circulation (q_{SL}) has been documented to be highly adaptable, capable of buffering 25%-60% of decreased portal flow [26, 27], and is a source of within-individual variability. Similarly, the MTT—which tracks the average transit time of a d4-CA molecule through oral administration, intestinal absorption, first pass extraction, and uptake into systemic circulation—is another source of intra-individual/biological variability. In contrast, some CM-specific flow rate parameters showed high reproducibility, including the anatomic shunt flow rate (q_{PS} ICC=0.88), portal inflow to the liver (q_{PL} ICC=0.87), splanchnic arterial circulation (q_{SP} ICC=1.00), and hepatic return to systemic compartment (q_{LS} ICC=0.85). These flow-related parameters from the CM are less likely to be affected by test administration, enhance the test interpretation, and have strong potential to be used as biomarkers for liver function.

Lastly, the CM parameters showed strong potential to distinguish between liver disease etiologies as well as between advanced and early fibrosis stages. Parameter scans of q_{PS} and ER simulate the spillover of d4-CA from anatomic shunting and hepatic extraction. In both simulations, varying q_{PS} and ER led to significant changes in clearance curves and demonstrates strong potential for the CM to distinguish these contributions. While these simulations represent extreme scenarios designed to test the limits of the CM, distinguishing these relative contributions in practice warrants additional investigation and validation.

The CM provides an excellent fit to individual subject clearance data, correlates well with previously validated measures of liver function, and provides highly reproducible indices of hepatic disease/health status with the potential to enhance understanding of liver function and physiology through estimation of hepatic extraction and anatomic shunting parameters. Novel information in the form of individualized pharmacokinetic parameters may serve as biomarkers for liver function and may ultimately improve the diagnostic performance and enhance the clinical utility and interpretation of the HepQuant test. Further, the CM developed here may provide new insights into how the mechanisms of liver function vary across a spectrum of chronic liver diseases and their severities, including HCV, NASH, primary sclerosing cholangitis, alcoholic liver disease, hepatocellular cancer, and polycystic liver disease. The individualized PK parameters estimated from this CM, when combined with artificial intelligence or machine learning algorithms, have significant potential to enhance the diagnostic performance and clinical utility of the HepQuant test of global liver function. This CM shows promise as a precision liver diagnostic tool by identifying which elements of liver function and physiology are affected by treatments or interventions.

The embodiments described in one aspect of the present disclosure are not limited to the aspect described. The embodiments may also be applied to a different aspect of the disclosure as long as the embodiments do not prevent these aspects of the disclosure from operating for its intended purpose

### EXAMPLES

### Example 1. A Multi-Compartmental Model of the HepQuant SHUNT Test Quantifies Anatomic Shunting and Stratifies Risk for Varices Using Combined Results from the HALT-C and SHUNT-V Studies

A compartmental model was developed using HepQuant SHUNT test results from the HALT-C Trial [Lee, W. M., et al., Evolution of the HALT-C Trial: pegylated interferon as maintenance therapy for chronic hepatitis C in previous interferon nonresponders. Controlled Clinical Trials, 2004. 25(5): p. 472-492; Everson, G. T., et al., The spectrum of hepatic functional impairment in compensated chronic hepatitis C: results from the Hepatitis C Anti-viral Long term Treatment against Cirrhosis Trial 1. Alimentary Pharmacology & Therapeutics, 2008. 27(9): p. 798-809], the SHUNT-V Study [The SHUNT-V Study for Varices, ClinicalTrials.gov study ID: NCT03583996], and a cohort of controls as follows: N=492 CLD patients with EGD (upper endoscopy, esophagogastroduodenoscopy) findings: 291 no varices, 143 small varices, 58 large varices; and N=26 lean controls.

A diagram of the Compartmental Model is shown in FIG. 1 illustrating the compartmental model of dual cholate clearance according to the present disclosure describing the transfer of cholate between systemic, portal, and liver compartments. D_{IV}=intravenous dose of 13C cholate; D_{PO}=oral dose of d4-CA; q_{SP}=splanchnic arterial flow rate; q_{PS}=anatomic shunt flow rate; q_{PL}=portal venous flow rate; q_{LS}=hepatic return to systemic circulation flow rate; q_{SL}=hepatic arterial flow rate; Cl_{H}=hepatic clearance.

Transfer between compartments was modeled by system of 18 differential equations with assumptions from measured and literature-derived values.

Model parameters were solved for each subject estimated by nonlinear least-squares regression.

Simulations and parameter estimation was solved for IV and oral plasma cholate concentration-time data simultaneously.

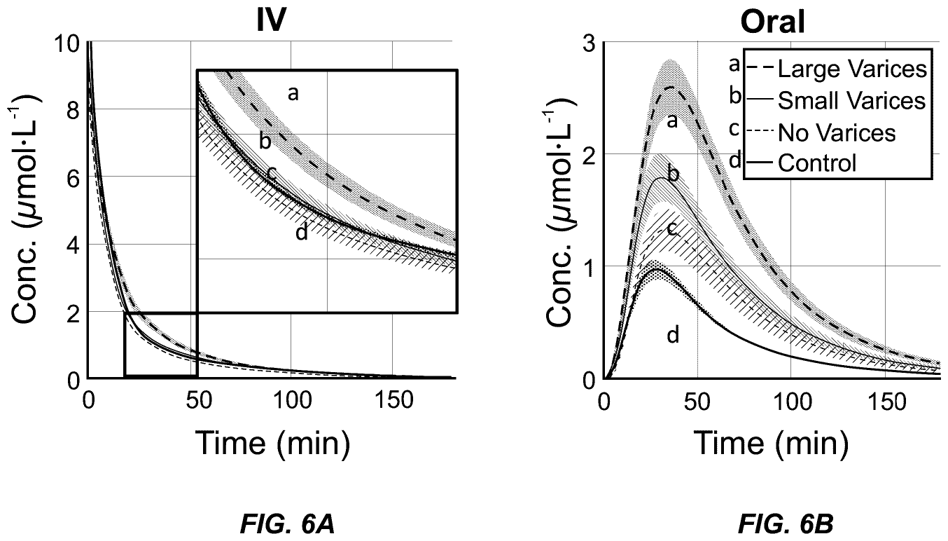

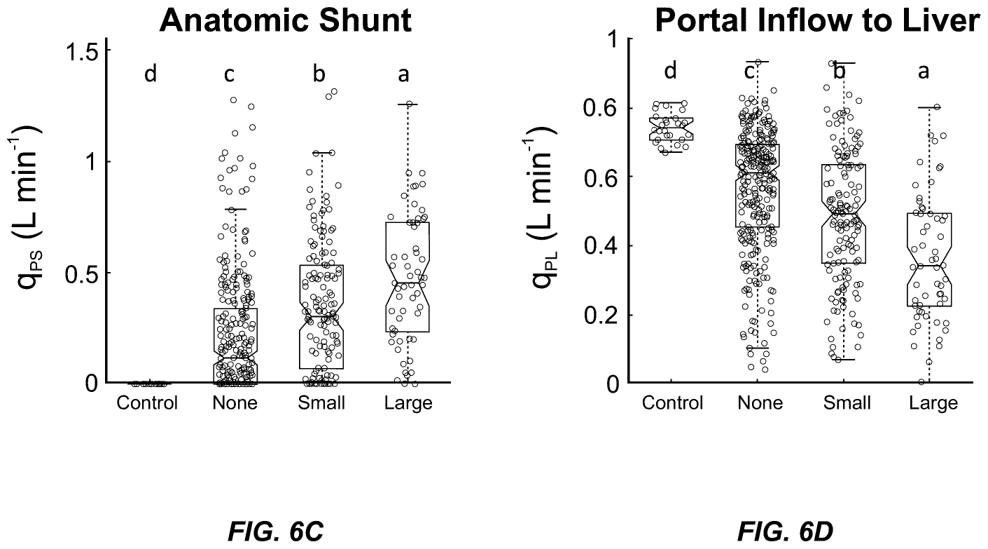

Compartmental Model results are shown in FIGS. 6A-D. A correlation between CM and MM data fits for DSI is shown in FIG. 7.

FIG. 6A shows a graph of Compartmental model fit of the data with median adjusted R² of 0.99 for 13C-CA IV curves in patients with large varices, small varices, no varices, or control subjects. Significant differences were observed in averaged oral and IV curves between controls, none, small, and large varices. Results suggest the HepQuant cholate SHUNT test is detecting the presence and the size/magnitude of anatomic shunts in patients with varices.

FIG. 6B shows a graph of Compartmental model fit of the data with median adjusted R² of 0.95 for d4-CA oral curves in patients with large varices, small varices, no varices, or control subjects. Significant differences were observed in averaged oral and IV curves between controls, none, small, and large varices. Results suggest the HepQuant cholate SHUNT test is detecting the presence and the size/magnitude of anatomic shunts in patients with varices.

FIG. 6C shows a graph of the median anatomic shunt flow rate (q_{PS}) in patients with large varices, small varices, no varices, and control subjects. Significant differences were demonstrated between all groups (Wilcoxon rank-sum p<0.05).

FIG. 6D shows a graph of portal inflow to the liver (q_{PL}) in patients with large varices, small varices, no varices, and control subjects. Reduced portal inflow to the liver (q_{PL}) was observed among varices groups, all groups p<0.001.

FIG. 7 shows a graph of DSI correlation between DSI values from the Compartmental Model data fit compared to Minimal Model data fit of cholate SHUNT test data in patients with large varices, small varices, no varices, and control subjects. The Compartmental Model DSI correlated well to Minimal Model DSI-a previously validated measure of global liver function.

The HepQuant Compartmental Model fit of cholate SHUNT data discriminated patients with small and large varices and attributed cholate clearance to hepatocyte function and anatomic shunting.

The HepQuant Compartmental Model also correlated with validated indices of hepatic disease. The present Compartmental analysis has significant potential to enhance diagnostic performance and clinical utility of HepQuant's cholate SHUNT global liver function tests.

### Clauses

Clause 1. A method for assessing liver function in a subject having or suspected of having or contracting a liver disease, comprising
obtaining blood or serum sample concentration data of an orally administered first distinguishable cholate compound in samples collected from a subject at a multiplicity of time points after oral administration;
obtaining blood or serum sample concentration data of an intravenously administered second distinguishable cholate compound in the samples collected from the subject at the multiplicity of time points after simultaneous intravenous co-administration;
fitting the first and second distinguishable cholate concentration data to a physiological-based compartmental model of dual cholate clearance to obtain fitted data, the compartmental model comprising body mass index (BMI), body weight (BW), and hematocrit (Hct) input values in the subject; and
calculating one or more indices of hepatic disease in the subject using the fitted data, wherein the one or more indices is associated with liver function in the subject.

Clause 2. The method of clause 1, further comprising
providing the one or more indices of hepatic disease to a medical professional for the purpose of developing a treatment plan in the subject.

Clause 3. The method of clause 1 or 2, wherein the one or more indices of hepatic disease is employed for a purpose selected from the group consisting of determining a need for treatment, predicting response to treatment, monitoring the effectiveness of a treatment, and predicting risk of clinical outcome in the subject.

Clause 4. The method of any one of clauses 1 to 3, wherein the physiological-based compartmental model comprises estimating
compartment volumes of a plurality of compartments in the subject;
flow parameters between the plurality of compartments in the subject; and
hepatic extraction of the intravenously administered distinguishable cholate in the subject.

Clause 5. The method of any one of clauses 1 to 4, wherein the physiological-based compartmental model further comprises estimating cholate binding and dose administration in the subject.

Clause 6. The method of any one of clauses 1 to 5, wherein the plurality of compartments in the subject comprises systemic, portal, and liver compartments.

Clause 7. The method of any one of clauses 1 to 6, wherein the estimating compartment volumes of a plurality of compartments in the subject comprises
estimating the systemic compartment volume (V_{S}), the portal compartment volume (V_{P}), and the liver compartment volume (V_{L}) in the subject, each in liters (L).

Clause 8. The method of any one of clauses 1 to 7, wherein the estimating of the systemic compartment volume (V_{S}) comprises

V_{S}=TBV·fₚₗₐₛₘₐ, wherein
TBV is total blood volume in the subject:

$$T B V = \frac{0.07 \cdot BW}{\sqrt{BMI / 22}} ,$$

wherein
BMI is body mass index (kg/m²) in the subject, and
BW is body weight (kg) in the subject; and
fₚₗₐₛₘₐ is the plasma fraction in the subject: fₚₗₐₛₘₐ=(1−Hct), wherein
Hct is the hematocrit in the subject.

Clause 9. The method of any one of clauses 1 to 8, wherein the estimating of the portal compartment volume (V_{P}) comprises

V_{P}=0.25·V_{S}.

Clause 10. The method of any one of clauses 1 to 9, wherein the estimating of the liver compartment volume (V_{L}) comprises

V_{L}=(0.275·22.46·BW·d_{L}·fₚₗₐₛₘₐ)/1000,
wherein d_{L} is Liver tissue density of 1.06 g·mL⁻¹.

Clause 11. The method of any one of clauses 1 to 10, wherein estimating the flow parameters between the plurality of compartments in the subject comprises
describing the intravenous and oral distinguishable cholate concentrations in each of the systemic (C_{S}), portal (C_{P}), and liver (C_{L}) compartments.

Clause 12. The method of any one of clauses 1 to 11, wherein the describing comprises calculating initial estimate of total hepatic inflow (Q_{L, init}), total hepatic inflow (Q_{L}), splanchnic arterial circulation (q_{SP}), hepatic portal venous inflow to the liver (q_{PL}), total hepatic venous return flow to systemic circulation (q_{LS}), hepatic arterial inflow to the liver (q_{SL}), and anatomic shunt flow (q_{PS}) rates in the subject.

Clause 13. The method of any one of clauses 1 to 12, wherein the calculating initial estimate of total hepatic inflow to the liver (Q_{L, init}) comprises

Q_{L,init}=1·w_{L}·fₚₗₐₛₘₐ, (L·min⁻¹·kg⁻¹), wherein
w_{L} is total liver weight (blood weight plus parenchymal weight): w_{L}=22.46·BW·d_{L}.

Clause 14. The method of any one of clauses 1 to 13, wherein the calculating total hepatic inflow to the liver (Q_{L}) comprises

Q_{L}=q_{SL}+q_{PL}, (L·min⁻¹)
wherein
   q_{SL} is rate of hepatic arterial inflow to the liver,

q_{SL}=0.25·Q_{L, init} (L·min⁻¹); and
q_{PL} is rate of portal venous inflow to the liver,

q_{PL}=q_{SP}−q_{PS} (L·min⁻¹), wherein
q_{SP} is splanchnic arterial blood flow rate to abdominal intestinal organs,

q_{SP}=0.75·Q_{L, init} (L·min⁻¹), and
q_{PS} is anatomic shunt flow rate, (L·min⁻¹) which bypasses the liver.

Clause 15. The method of any one of clauses 1 to 14, wherein the calculating total hepatic venous return flow rate to systemic circulation (q_{LS}) comprises: q_{LS}=q_{PL}+q_{SL}.

Clause 16. The method of any one of clauses 1 to 15, wherein estimating the hepatic extraction in the subject comprises calculating a fast phase extraction ratio (ER_{fast}), fast phase clearance of intravenously administered distinguishable cholate (Cl_{IV}), and slow phase of hepatic clearance (Cl_{H}) in the subject.

Clause 17. The method of any one of clauses 1 to 16, wherein the fast phase extraction ratio (ER^{fast}) is calculated as the ratio of total IV clearance (Cl_{IV}) (0 to 180 min) to first phase clearance (Cl_{FP}) (0-20 min)

$$E {R}_{fast} = \frac{{Cl}_{IV}}{{Cl}_{F P}} = \frac{{C}_{0}}{{k}_{fast} \cdot {AUC}_{IV}} ,$$

wherein
AUC_{IV} is the area under the IV curve;
k_{fast} is estimated from the slope of log concentration-time IV curves between 5- and 20-minute timepoints,

$${k}_{fast} = - \frac{ln ( C [ 20 ] ) - ln ( C [ 5 ] )}{t ( 20 ) - t ( 5 )} ( {min}^{- 1} ) ;$$

and
and C₀ is the extrapolated initial intravenously administered distinguishable cholate concentration

$${C}_{0} = - \frac{C ( 5 )}{{e}^{- {k}_{fast} \cdot t ( 5 )}} ( μ mol \cdot {L}^{- 1} ) ,$$

wherein C(5) is the concentration of intravenously administered distinguishable cholate in the sample collected at 5 minutes after administration.

Clause 18. The method of any one of clauses 1 to 17, wherein the fast phase clearance of intravenously administered distinguishable cholate (Cl_{IV}) is calculated as

Cl_{IV}=Q_{L}·ER_{fast} (L·min⁻¹).

Clause 19. The method of any one of clauses 1 to 18, wherein the slow phase of hepatic clearance (Cl_{H}) in the subject is the hepatic clearance of orally administered distinguishable cholate and slow phase of intravenously administered distinguishable cholate clearance in the subject, optionally calculated as

Cl_{H}=Q_{L}·ER_{slow} (L·min⁻¹).

Clause 20. The method of any one of clauses 1 to 19, wherein the estimating cholate binding in the subject comprises modeling (1) albumin-bound noncellular intravascular distinguishable cholate, (2) extravascular distinguishable cholate distributed to cells/tissues, and (3) free/unbound distinguishable cholate.

Clause 21. The method of any one of clauses 1 to 20, wherein the modeling of albumin-bound noncellular intravascular distinguishable cholate comprises estimating an initial albumin-bound noncellular intravascular fraction of total dose estimate (f_{A, init}) comprising calculating the ratio of systemic compartment volume (Vs) to the volume of distribution (Vd)

f_{A, init}=V_{S}/V_{d},
wherein V_{d}=D_{IV}/C₀ (L), D_{IV} is intravenous dose of distinguishable cholate, and C₀ and V_{S} are as defined above.

Clause 22. The method of any one of clauses 1 to 21, wherein the actual intravascular fraction (f_{A}) is determined comprising parameter estimation comprising weighted nonlinear least-squares regression at each distinguishable cholate concentration.

Clause 23. The method of any one of clauses 1 to 22, wherein the weights of the weighted nonlinear least-squares regression are reduced to reflect higher uncertainty during rapid distribution phase of IV clearance, and at low concentrations of oral clearance, optionally wherein the 5 min IV distinguishable cholate weight is 0.33, and the 5 min and 90 min oral distinguishable cholate weights are 0.67.

Clause 24. The method of any one of clauses 1 to 22, wherein the one or more indices of hepatic disease is selected from the group consisting of portal hepatic filtration rate (HFRp), systemic hepatic filtration rate (HFRs), cholate SHUNT, STAT, liver disease severity index (DSI), and hepatic reserve (HR) in the subject.

Clause 25. The method of any one of clauses 1 to 24, wherein the portal hepatic filtration rate (HFRp) is a clearance adjusted for body weight (BW) of the subject

$$H F {R}_{P} = \frac{{D}_{P O}}{A U {C}_{P O} \cdot BW} ,$$

wherein
D_{PO} is oral dose of the distinguishable cholate;
AUC_{PO} is area under the oral distinguishable cholate blood or serum sample concentration curve over the multiplicity of time points after administration.

Clause 26. The method of any one of clauses 1 to 25, wherein the systemic hepatic filtration rate (HFRs) is a clearance adjusted for body weight (BW) of the subject

$${HFR}_{S} = \frac{{D}_{IV}}{{AUC}_{IV} \cdot BW} ,$$

wherein
D_{IV} is intravenous dose of the distinguishable cholate;
AUC_{IV} is area under the intravenous distinguishable cholate blood or serum sample concentration curve over the multiplicity of time points after administration.

Clause 27. The method of any one of clauses 1 to 26, wherein the cholate SHUNT (F) is a comparison of bioavailability of orally administered distinguishable cholate with bioavailability of IV-administered distinguishable calculated by the ratio of areas under the oral and IV distinguishable cholate concentration curves (AUC_{PO} and AUC_{IV}, respectively) and normalized by the IV dose (D_{IV}) and oral dose (D_{PO}), optionally wherein the SHUNT is calculated comprising

$$F = \frac{{AUC}_{P O} \cdot {D}_{IV}}{{AUC}_{IV} \cdot {D}_{P O}} .$$

Clause 28. The method of any one of clauses 1 to 27, wherein the STAT is a single point blood or serum oral distinguishable cholate estimate of liver disease severity index (DST).

Clause 29. The method of any one of clauses 1 to 28, wherein the DSI is a score indicative of overall liver function comprising both portal and systemic HFR, optionally wherein

$$DSI = A \cdot \sqrt{{( ln [ \frac{{HFR}_{P , max}}{{HFR}_{P}} ] )}^{2} + {( ln [ \frac{{HFR}_{S , max}}{{HFR}_{S}} ] )}^{2}} ,$$

wherein HFR_{P,max} and HFR_{S,max} are the upper limits of clearance of healthy controls, and A is a factor to scale DSI score from 0 to 50.

Clause 30. The method of any one of clauses 1 to 29, wherein the hepatic reserve (HR) is a numerical index representing overall hepatic health with HR values between 0 and 100

$$H R = 100 - A \cdot \sqrt{{( ln [ \frac{{HFR}_{P , lean}}{{HFR}_{P}} ] )}^{2} + {( ln [ \frac{{HFR}_{S , lean}}{{HFR}_{S}} ] )}^{2}} ,$$

wherein
HFR_{P} and HFR_{S} are indexed to lean controls minus one standard deviation (HFR_{P,lean} and HFR_{S,lean}); and A is a constant integer from 100 to 0.

Clause 31. The method of any one of clauses 1 to 30, wherein the first distinguishable cholate compound is a first stable isotope labeled cholate and the second distinguishable cholate compound is a second stable isotope labeled cholate.

Clause 32. The method of any one of clauses 1 to 31, wherein the first and second stable isotope labeled cholates are selected from 2,2,4,4-d4 cholate and 24-¹³C-cholate.

Clause 33. The method of any one of clauses 1 to 32, further comprising
receiving a plurality of blood or serum samples collected from the subject having or suspected of having or contracting a chronic liver disease, following oral administration of a dose of the first distinguishable cholate compound (DPO) to the subject and simultaneous intravenous co-administration of a dose of a second distinguishable cholate compound (DIV) to the subject, wherein the samples have been collected over intervals spanning a period of time after administration; and
quantifying the concentration of the first and the second distinguishable cholate compounds in each sample.

Clause 34. The method of any one of clauses 1 to 33, wherein the fitting comprises
generating an individualized oral clearance curve from the concentration of the first distinguishable cholate compound in each sample comprising using a computer algorithm curve fitting to the compartmental model and computing the area under the individualized oral clearance curve (AUC_{PO});
generating an individualized intravenous clearance curve from the concentration of the second distinguishable cholate compound in each sample by use of a computer algorithm curve fitting to the compartmental model and computing the area under the individualized intravenous clearance curve (AUC_{IV}).

Clause 35. The method of any one of clauses 1 to 34, wherein the samples have been collected from the subject over intervals comprising from one to seven time points, two to six time points, or three to five time points after administration.

Clause 36. The method of any one of clauses 1 to 35, wherein the samples have been collected from the patient at about 0, about 5, about 20, about 45, about 60, and about 90 minutes after administration.

Clause 37. The method of any one of clauses 1 to 36, wherein the subject is a human subject.

Clause 38. The method of any one of clauses 1 to 37, wherein the liver disease is a chronic liver disease selected from the group consisting of chronic hepatitis C (CHC), chronic hepatitis B, alcoholic liver disease, Alcoholic SteatoHepatitis (ASH), and Non-Alcoholic Fatty Liver Disease (NAFLD), steatosis, Non-Alcoholic SteatoHepatitis (NASH), autoimmune liver disease, cryptogenic cirrhosis, hemochromatosis, Wilson's disease, alpha-1-antitrypsin deficiency, liver cancer, liver failure, cirrhosis, primary sclerosing cholangitis (PSC), and other cholestatic liver diseases.

Clause 39. The method of any one of clauses 1 to 38, wherein the clinical outcome is selected from the group consisting of Child-Turcotte-Pugh (CTP) progression, Model for End-stage Liver Disease (MELD) progression, variceal hemorrhage, ascites, splenomegaly, varices, portal hypertension (PHTN), hepatic encephalopathy, hepatocellular carcinoma (HCC), decompensation, or liver-related death.

Clause 40. The method of any one of clauses 1 to 39, wherein the treatment is selected from the group consisting of antiviral treatments, antifibrotic treatments, antibiotics, immunosuppressive treatments, anti-cancer treatments, ursodeoxycholic acid, farnesoid X receptor ligands, insulin sensitizing agents, interventional treatment, liver transplant, lifestyle changes, dietary restrictions, low glycemic index diet, antioxidants, vitamin supplements, transjugular intrahepatic portosystemic shunt (TIPS), catheter-directed thrombolysis, balloon dilation and stent placement, balloon-dilation and drainage, weight loss, exercise, and avoidance of alcohol.

Clause 41. The method of any one of any one of clauses 1 to 40, wherein monitoring the need for treatment in the subject comprises
determining the one or more indices of hepatic disease in the subject; and
comparing the one or more indices of hepatic disease to one or more cutoff value(s),
wherein a change in the one or more indices of hepatic disease compared to cutoff value(s) is indicative of the need for treatment in the subject.

Clause 42. The method of any one of clauses 1 to 41, wherein the cutoff value(s) are derived from one or more normal healthy controls, a group of known patients, or within the subject over time.

Clause 43. The method of any one of clauses 1 to 42, wherein the group of known patients is suffering from a disease or condition selected from the group consisting of a chronic liver disease having a fibrosis stage; portal hypertension; Childs-Turcotte-Pugh (CTP) score A; CTP score B, CTP score C; Model for End-stage Liver Disease (MELD) progression score, primary sclerosing cholangitis (PSC) not listed for transplant; PSC listed for liver transplant; PSC listed for liver transplant without varices; PSC listed for liver transplant with varices; ascites; stomal bleeding; splemomegaly; varices; large varices, variceal hemorrhage; hepatic encephalopathy, decompensation; or liver disease-related death.

Clause 44. The method of any one of clauses 1 to 43, wherein the fibrosis stage is determined by a method comprising liver biopsy or elastography.

Clause 45. The method of any one of clauses 1 to 44, wherein the liver biopsy determines Ishak fibrosis score (liver biopsy) of F2 (mild portal fibrosis), F3, F4 (moderate bridging fibrosis), F5 (nodular formation and incomplete cirrhosis), or F6 (cirrhosis).

Clause 46. The method of any one of clauses 1 to 45, wherein the anatomic shunt flow rate (q_{PS}) differentiates healthy controls from subjects with large varices.

Clause 47. The method of any one of clauses 1 to 46, wherein the rate of portal venous inflow to the liver (q_{PL}) differentiates healthy controls, subjects with no varices, subjects with small varices, and subjects with large varices.

Clause 48. The method of any one of clauses 1 to 47, wherein the physiological-based compartmental model comprises an algorithm estimating in the systemic compartment: intravenously administered distinguishable cholate that is albumin bound; intravenously administered distinguishable cholate that is bound/distributed to cells/tissues; and intravenously administered distinguishable cholate that is unbound/free; the algorithm optionally comprising

### Albumin Bound:

$$\frac{d {C}_{S , AIV}}{dt} = \frac{1}{{V}_{S}} ( {f}_{A} \cdot {D}_{IV} - [ {q}_{S P} + {q}_{S L} ] {C}_{S , AIV} + {q}_{P S} \cdot {C}_{P , AIV} + {q}_{L S} \cdot {C}_{L , AIV} ) \qquad ( 1 ’ )$$

### Bound/Distributed to Cells/Tissues:

$$\frac{d {C}_{S , BIV}}{dt} = \frac{1}{{V}_{S}} ( [ 1 - {f}_{A} ] {D}_{IV} - [ {q}_{S P} + {q}_{S L} ] {C}_{S , BIV} + {q}_{P S} \cdot {C}_{P , BIV} + {q}_{LS} \cdot {C}_{L , BIV} ) \qquad ( 2 ’ )$$

### Unbound/Free Cholate:

$$\frac{d {C}_{S , UIV}}{dt} = \frac{1}{{V}_{S}} ( - [ {q}_{S P} + {q}_{S L} ] {C}_{S , UIV} + {q}_{P S} \cdot {C}_{P , UIV} + {q}_{LS} \cdot {C}_{L , UIV} ) . \qquad ( 3 ’ )$$

Clause 49. The method of any one of clauses 1 to 48, wherein the physiological-based compartmental model comprises an algorithm estimating in the systemic compartment: orally administered distinguishable cholate that is albumin bound; orally administered distinguishable cholate that is bound/distributed to cells/tissues; and orally administered distinguishable cholate that is unbound/free; the algorithm optionally comprising

### Albumin Bound:

$$\frac{d {C}_{S , APO}}{dt} = \frac{1}{{V}_{S}} ( - [ {q}_{S P} + {q}_{S L} ] {C}_{S , APO} + {q}_{P S} \cdot {C}_{P , APO} + {q}_{L S} \cdot {C}_{L , APO} ) \qquad ( 4 ’ )$$

### Bound/Distributed to Cells/Tissues:

$$\frac{{dC}_{S , BPO}}{dt} = \frac{1}{{V}_{S}} ( - [ {q}_{S P} + {q}_{S L} ] {C}_{S , BPO} + {q}_{P S} \cdot {C}_{P , BPO} + {q}_{LS} \cdot {C}_{L , BPO} ) \qquad ( 5 ’ )$$

### Unbound/Free Cholate:

$$\frac{d {C}_{S , UPO}}{dt} = \frac{1}{{V}_{S}} ( - [ {q}_{S P} + {q}_{S L} ] {C}_{S , U P O} + {q}_{P S} \cdot {C}_{P , U P O} + {q}_{LS} \cdot {C}_{L , U P O} ) . \qquad ( 6 ’ )$$

Clause 50. The method of any one of clauses 1 to 49, wherein the physiological-based compartmental model comprises an algorithm estimating in the portal compartment: intravenously administered distinguishable cholate that is albumin bound; intravenously administered distinguishable cholate that is bound/distributed to cells/tissues; and intravenously administered distinguishable cholate that is unbound/free; the algorithm optionally comprising

### Albumin Bound:

$$\frac{d {C}_{P , AIV}}{d t} = \frac{1}{{V}_{P}} ( {q}_{S P} \cdot {C}_{S , AIV} - [ {q}_{P L} + {q}_{P S} ] {C}_{P , AIV} ) \qquad ( 7 ’ )$$

Bound/Distributed to Cells/Tissues:

$$\frac{d {C}_{P , BIV}}{d t} = \frac{1}{{V}_{P}} ( {q}_{S P} \cdot {C}_{S , BIV} - [ {q}_{P L} + {q}_{P S} ] {C}_{P , BIV} ) \qquad ( 8 ’ )$$

### Unbound/Free Cholate:

$$\frac{d {C}_{P , UIV}}{d t} = \frac{1}{{V}_{P}} ( {q}_{S P} \cdot {C}_{S , UIV} - [ {q}_{P L} + {q}_{P S} ] {C}_{P , UIV} ) . \qquad ( 9 ’ )$$

Clause 51. The method of any one of clauses 1 to 50, wherein the physiological-based compartmental model comprises an algorithm estimating in the portal compartment orally administered distinguishable cholate that is albumin bound; orally administered distinguishable cholate that is bound/distributed to cells/tissues; and orally administered distinguishable cholate that is unbound/free; the algorithm optionally comprising

### Albumin Bound:

$$\frac{d {C}_{P , APO}}{d t} = \frac{1}{{V}_{P}} ( {f}_{A} \cdot {D}_{P O} + {q}_{S P} \cdot {C}_{S , APO} - [ {q}_{P L} + {q}_{P S} ] {C}_{P , APO} ) \qquad ( 10 ’ )$$

### Bound/Distributed to Cells/Tissues:

$$\frac{d {C}_{P , BPO}}{d t} = \frac{1}{{V}_{P}} ( [ 1 - {f}_{A} ] {D}_{P O} + {q}_{S P} \cdot {C}_{S , BPO} - [ {q}_{P L} + {q}_{P S} ] {C}_{P , BPO} ) \qquad ( 11 ’ )$$

### Unbound/Free Cholate:

$$\frac{d {C}_{P , UPO}}{d t} = \frac{1}{{V}_{P}} ( {q}_{S P} \cdot {C}_{S , U P O} - [ {q}_{P L} + {q}_{P S} ] {C}_{P , U P O} ) . \qquad ( 12 ’ )$$

Clause 52. The method of any one of clauses 1 to 51, wherein the physiological-based compartmental model comprises an algorithm estimating in the liver compartment: intravenously administered distinguishable cholate that is albumin bound; intravenously administered distinguishable cholate that is bound/distributed to cells/tissues; and intravenously administered distinguishable cholate that is unbound/free; the algorithm optionally comprising

### Albumin Bound:

$$\frac{{dC}_{L , AIV}}{dt} = \frac{1}{{V}_{L}} ( {k}_{BA} \cdot {C}_{L , BIV} \cdot {V}_{L} - [ {k}_{AU} \cdot {V}_{L} + {q}_{LS} ] {C}_{L , AIV} + {q}_{PL} \cdot {C}_{P , AIV} + {q}_{SL} \cdot {C}_{S , AIV} ) \qquad ( {13}^{′} )$$

### Bound/Distributed to Cells/Tissues:

$$\frac{{dC}_{L , BIV}}{dt} = \frac{1}{{V}_{L}} ( - [ {k}_{BA} \cdot {V}_{L} + {q}_{LS} ] {C}_{L , BIV} + {q}_{PL} \cdot {C}_{P , BIV} + {q}_{SL} \cdot {C}_{S , BIV} ) \qquad ( {14}^{′} )$$

### Unbound/Free Cholate:

$$\frac{{dC}_{L , UIV}}{dt} = \frac{1}{{V}_{L}} ( {k}_{AU} \cdot {C}_{L , AIV} \cdot {V}_{L} - [ {Cl}_{IV} + {q}_{LS} ] {C}_{L , UIV} + {q}_{PL} \cdot {C}_{P , UIV} + {q}_{SL} \cdot {C}_{S , UIV} ) . \qquad ( {15}^{′} )$$

Clause 53. The method of any one of clauses 1 to 52, wherein the physiological-based compartmental model comprises an algorithm estimating in the liver compartment: orally administered distinguishable cholate that is albumin bound; orally administered distinguishable cholate that is bound/distributed to cells/tissues; and orally administered distinguishable cholate that is unbound/free; the algorithm optionally comprising

### Albumin Bound:

$$\frac{{dC}_{L , APO}}{dt} = \frac{1}{{V}_{L}} ( {k}_{BA} \cdot {C}_{L , BPO} \cdot {V}_{L} - [ {k}_{AU} \cdot {V}_{L} + {q}_{LS} ] {C}_{L , APO} + {q}_{PL} \cdot {C}_{P , APO} + {q}_{SL} \cdot {C}_{S , APO} ) \qquad ( {16}^{′} )$$

### Bound/Distributed to Cells/Tissues:

$$\frac{{dC}_{L , BPO}}{dt} = \frac{1}{{V}_{L}} ( - [ {k}_{BA} \cdot {V}_{L} + {q}_{LS} ] {C}_{L , BPO} + {q}_{PL} \cdot {C}_{P , BPO} + {q}_{SL} \cdot {C}_{S , BPO} ) \qquad ( {17}^{′} )$$

### Unbound/Free Cholate:

$$\frac{{dC}_{L , UPO}}{dt} = \frac{1}{{V}_{L}} ( {k}_{AU} \cdot {C}_{L , APO} \cdot {V}_{L} - [ {Cl}_{H} + {q}_{LS} ] {C}_{L , UPO} + {q}_{PL} \cdot {C}_{P , UPO} + {q}_{SL} \cdot {C}_{S , UPO} ) . \qquad ( {18}^{′} )$$

### REFERENCES

[1] Spengler E K, Loomba R. Recommendations for diagnosis, referral for liver biopsy, and treatment of Nonalcoholic Fatty Liver Disease and Nonalcoholic Steatohepatitis. Mayo Clin Proc. 2015; 90:1233-46.
[2] Estes C, Razavi H, Loomba R, Younossi Z, Sanyal A J. Modeling the epidemic of nonalcoholic fatty liver disease demonstrates an exponential increase in burden of disease. Hepatology. 2018; 67:123-33.
[3] Schweitzer A, Horn J, Mikolajczyk R T, Krause G, Ott J J. Estimations of worldwide prevalence of chronic hepatitis B virus infection: a systematic review of data published between 1965 and 2013. Lancet. 2015; 386:1546-55.
[4] Edlin B R, Eckhardt B J, Shu M A, Holmberg SD, Swan T. Toward a more accurate estimate of the prevalence of hepatitis C in the United States. Hepatology. 2015; 62:1353-63.
[5] Wong T, Dang K, Ladhani S, Singal A K, Wong R J. Prevalence of alcoholic fatty liver disease among adults in the United States, 2001-2016. JAMA. 2019; 321:1723-5.
[6] Lu M, Li J, Haller I V, Romanelli R J, VanWormer J J, Rodriguez C V, et al. Factors associated with prevalence and treatment of primary biliary cholangitis in United States Health Systems. Clin Gastroenterol Hepatol. 2018; 16:1333-41.e6.
[7] Boonstra K, Beuers U, Ponsioen C Y. Epidemiology of primary sclerosing cholangitis and primary biliary cirrhosis: A systematic review. J Hepatol. 2012; 56:1181-8.
[8] Peery A F, Crockett S D, Barritt A S, Dellon E S, Eluri S, Gangarosa L M, et al. Burden of gastrointestinal, liver, and pancreatic diseases in the United States. Gastroenterology. 2015; 149:1731-41.
[9] SAMEISA, Center for Behavioral Health Statistics and Quality, National Survey on drug use and health, 2017 and 2018.
[10] National Center for Health Statistics. Health, United States, 2016: With chartbook on long-term trends in health. Hyattsville, MD. 2017. https://www.cdc.gov/nchs/data/hus/husl6.pdf.
[11] Burton J R, Helmke S, Lauriski S, Kittelson J, Everson G T. The within-individual reproducibility of the disease severity index from the HepQuant SHUNT test of liver function and physiology. Transl Res. 2021; 233:5-15.
[12] Helmke S, Colmenero J, Everson G T. Noninvasive assessment of liver function. Curr Opin Gastroenterol. 2015; 31.
[13] ClinicalTrials.gov. The SHUNT-V Study for Varices.
[14] Everson G T, Martucci M A, Shiffman M L, Sterling R K, Morgan T R, Hoefs J C, et al. Portal-systemic shunting in patients with fibrosis or cirrhosis due to chronic hepatitis C: the minimal model for measuring cholate clearances and shunt. Aliment Pharmacol Ther. 2007; 26:401-10.
[15] Lemmens H J, Bernstein D P, Brodsky J B. Estimating blood volume in obese and morbidly obese patients. Obes Surg. 2006; 16:773-6.
[16] Mathew J, Sankar P, Varacallo M. Physiology, Blood Plasma. StatPearls. Treasure Island (FL): StatPearls Publishing; 2021.
[17] Eipel C, Abshagen K, Vollmar B. Regulation of hepatic blood flow: the hepatic arterial buffer response revisited. World J Gastroenterol. 2010; 16:6046-57.
[18] Lautt W W. Chapter 2, Overview. Hepatic Circulation: Physiology and Pathophysiology. San Rafael (CA): Morgan & Claypool Life Sciences; 2009.
[19] Andersen V, Sonne J, Sletting S, Prip A. The volume of the liver in patients correlates to body weight and alcohol consumption. Alcohol Alcohol. 2000; 35:531-2.
[20] Yu HC, You H, Lee H, Jin Z W, Moon J I, Cho B H. Estimation of standard liver volume for liver transplantation in the Korean population. Liver Transpl. 2004; 10:779-83.
[21] Heinemann A, Wischhusen F, Piischel K, Rogiers X. Standard liver volume in the Caucasian population. Liver Transpl Surg. 1999; 5:366-8.
[22] Lautt W W. Hepatic vasculature: a conceptual review. Gastroenterology. 1977; 73:1163-9.
[23] Rappaport A M. Hepatic blood flow: morphologic aspects and physiologic regulation. Int Rev Physiol. 1980; 21:1-63.
[24] Harper D, Chandler B. Splanchnic circulation. BJA Education. 2016; 16:66-71.
[25] Tygstrup N, Winkler K, Mellemgaard K, Andreassen M. Determination of the hepatic arterial blood flow and oxygen supply in man by clamping the hepatic artery during surgery. J Clin Invest. 1962; 41:447-54.
[26] Lautt W W. Relationship between hepatic blood flow and overall metabolism: the hepatic arterial buffer response. Fed Proc. 1983; 42:1662-6.
[27] Lautt W W, Legare D J, d'Almeida M S. Adenosine as putative regulator of hepatic arterial flow (the buffer response). Am J Physiol Heart Circ Physiol. 1985; 248:H331-H8.
[28] Atkinson A J, Kushner W. Clinical Pharmacokinetics. Annu Rev Pharmacol Toxicol. 1979; 19:105-27.
[29] Rowland M, Tozer T N. Clinical Pharmacokinetics: Concepts and Applications: Williams & Wilkins; 1995.
[30] Hofmann A F. The Continuing Importance of Bile Acids in Liver and Intestinal Disease. Arch Intern Med. 1999; 159:2647-58.
[31] Gilmore I T, Thompson R P. Plasma clearance of oral and intravenous cholic acid in subjects with and without chronic liver disease. Gut. 1980; 21:123.
[32] O'Maille E R, Richards TG, Short A H. The influence of conjugation of cholic acid on its uptake and secretion: hepatic extraction of taurocholate and cholate in the dog. J Physiol. 1967; 189:337-50.
[33] Salem F, Abduljalil K, Kamiyama Y, Rostami-Hodjegan A. Considering Age Variation When Coining Drugs as High versus Low Hepatic Extraction Ratio. Drug Metab Dispos. 2016; 44:1099.
[34] Kwon Y. Chapter 2, Pharmacokinetic Study Design and Data Interpretation. Handbook of Essential Pharmacokinetics, Pharmacodynamics and Drug Metabolism for Industrial Scientists. Boston, MA: Springer US; 2002. p. 3-28.
[35] Wilkinson G R, Shand D G. A physiological approach to hepatic drug clearance. Clin Pharm Therap. 1975; 18:377-90.
[36] Shand D G, Cotham R H, Wilkinson G R. Perfusion-limited effects of plasma drug binding on hepatic drug extraction. Life Sci. 1976; 19:125-30.
[37] Tsao S C, Sugiyama Y, Sawada Y, Iga T, Hanano M. Kinetic analysis of albumin-mediated uptake of warfarin by perfused rat liver. J Pharmacokinet Biopharm. 1988; 16:165-81.
[38] Miyauchi S, Masuda M, Kim S-J, Tanaka Y, Lee K-R, Iwakado S, et al. The Phenomenon of Albumin-Mediated Hepatic Uptake of Organic Anion Transport Polypeptide Substrates: Prediction of the In Vivo Uptake Clearance from the In Vitro Uptake by Isolated Hepatocytes Using a Facilitated-Dissociation Model. Drug Metab Dispos. 2018; 46:259.
[39] Kwon Y. Chapter 4, Absorption. Handbook of Essential Pharmacokinetics, Pharmacodynamics and Drug Metabolism for Industrial Scientists. Boston, MA: Springer US; 2002. p. 35-72.
[40] Savic R M, Jonker D M, Kerbusch T, Karlsson M O. Implementation of a transit compartment model for describing drug absorption in pharmacokinetic studies. J Pharmacokinet Pharmacodyn. 2007; 34:711-26.
[41] Wilkins Justin J, Savic Radojka M, Karlsson Mats O, Langdon G, McIlleron H, Pillai G, et al. Population pharmacokinetics of rifampin in pulmonary tuberculosis patients, including a semimechanistic model to describe variable absorption. Antimicrob Agents Chemother. 2008; 52:2138-48.
[42] Everson G T, Shiffman M L, Morgan T R, Hoefs J C, Sterling R K, Wagner D A, et al. The spectrum of hepatic functional impairment in compensated chronic hepatitis C: results from the Hepatitis C Anti-viral Long-term Treatment against Cirrhosis Trial. Aliment Pharmacol Ther. 2008; 27:798-809.
[43] Helmke S M, DeSanto J, Herman A, Lauriski S, Everson G T. A disease severity index based on dual cholate clearances and shunt outperforms biopsy at predicting clinical outcomes in chronic Hepatitis C. Gastroenterology. 2013; 144:S-951-S-2.
[44] Helmke S, Kulig C, Lauriski S, Herman A, Dudekula A, Everson G. Significant alteration of the portal ciculation in over half of the chronic HCV patients with Ishak fibrosis stage F0-F2. Hepatology. 2011; 54.
[45] Koo T K, Li M Y. A Guideline of Selecting and Reporting Intraclass Correlation Coefficients for Reliability Research. J Chiropr Med. 2016; 15:155-63.
[46] McGraw K O, Wong S P. Forming inferences about some intraclass correlation coefficients. Psychol Methods. 1996; 1:30-46.
[47] Cornbleet P J, Gochman N. Incorrect least-squares regression coefficients in method-comparison analysis. Clin Chem. 1979; 25:432-8.
[48] Bland J M, Altman D G. Measuring agreement in method comparison studies. Stat Methods Med Res. 1999; 8:135-60.

What is claimed is:

1. A method for assessing liver function in a subject having or suspected of having or contracting a liver disease, comprising obtaining blood or serum sample concentration data of an orally administered first distinguishable cholate compound in samples collected from a subject at a multiplicity of time points after oral administration;
obtaining blood or serum sample concentration data of an intravenously administered second distinguishable cholate compound in the samples collected from the subject at the multiplicity of time points after simultaneous intravenous co-administration;
fitting the first and second distinguishable cholate concentration data to a physiological-based compartmental model of dual cholate clearance to obtain fitted data, the compartmental model comprising body mass index (BMI), body weight (BW), and hematocrit (Hct) input values in the subject; and
calculating one or more indices of hepatic disease in the subject using the fitted data, wherein the one or more indices is associated with liver function in the subject.

2. The method of claim 1, further comprising
providing the one or more indices of hepatic disease to a medical professional for the purpose of developing a treatment plan in the subject.

3. The method of claim 1, wherein the one or more indices of hepatic disease is employed for a purpose selected from the group consisting of determining a need for treatment, predicting response to treatment, monitoring the effectiveness of a treatment, and predicting risk of clinical outcome in the subject.

4. The method of claim 1, wherein the physiological-based compartmental model comprises estimating
compartment volumes of a plurality of compartments in the subject;
flow parameters between the plurality of compartments in the subject; and
hepatic extraction of the intravenously administered distinguishable cholate in the subject.

5. The method of claim 4, wherein the physiological-based compartmental model further comprises estimating cholate binding and dose administration in the subject.

6. The method of claim 4, wherein the plurality of compartments in the subject comprises systemic, portal, and liver compartments.

7. The method of claim 6, wherein the estimating compartment volumes of a plurality of compartments in the subject comprises
estimating the systemic compartment volume (V_{S}), the portal compartment volume (V_{P}), and the liver compartment volume (V_{L}) in the subject, each in liters (L).

8. The method of claim 7, wherein the estimating of the systemic compartment volume (V_{S}) comprises
V_{S}=TBV·fₚₗₐₛₘₐ, wherein
TBV is total blood volume in the subject:
$$T B V = \frac{0.07 \cdot B W}{\sqrt{B M I / \text{?}}} ,$$
$$\text{?} \text{indicates text missing or illegible when filed}$$
wherein
BMI is body mass index (kg/m²) in the subject; and
BW is body weight (kg) in the subject; and
fₚₗₐₛₘₐ is the plasma fraction in the subject: fₚₗₐₛₘₐ=(1−Hct), wherein
Hct is the hematocrit in the subject.

9. The method of claim 7 or 8,, wherein the estimating of the portal compartment volume (V_{P}) comprises
V_{P}=0.25·V_{S}.

10. The method of claim 7, wherein the estimating of the liver compartment volume (V_{L}) comprises
V_{L}=(0.275·22.46·BW·d_{L}·fₚₗₐₛₘₐ)/1000,
wherein d_{L} is Liver tissue density of 1.06 g·mL⁻¹.

11. The method of claim 4, wherein estimating the flow parameters between the plurality of compartments in the subject comprises
describing the intravenous and oral distinguishable cholate concentrations in each of the systemic (C_{S}), portal (C_{P}), and liver (C_{L}) compartments.

12. The method of claim 11, wherein the describing comprises calculating initial estimate of total hepatic inflow (Q_{L, init}), total hepatic inflow (Q_{L}), splanchnic arterial circulation (q_{SP}), hepatic portal venous inflow to the liver (q_{PL}), total hepatic venous return flow to systemic circulation (q_{LS}), hepatic arterial inflow to the liver (q_{SL}), and anatomic shunt flow (q_{PS}) rates in the subject.

13. The method of claim 12, wherein the calculating initial estimate of total hepatic inflow to the liver (Q_{L, init}) comprises
Q_{L,init}=1·w_{L}·fₚₗₐₛₘₐ, (L·min⁻¹·kg⁻¹), wherein
w_{L} is total liver weight (blood weight plus parenchymal weight): w_{L}=22.46·BW·d_{L}.

14. The method of claim 12 or 13, wherein the calculating total hepatic inflow to the liver (Q_{L}) comprises
Q_{L}=q_{SL}+q_{PL}, (L·min^{31 1})
wherein
q_{SL} is rate of hepatic arterial inflow to the liver,
q_{SL}=0.25·Q_{L, init} (L·min⁻¹); and
q_{PL} is rate of portal venous inflow to the liver,
q_{PL}=q_{SP}−q_{PS} (L·min⁻¹), wherein
q_{SP} is splanchnic arterial blood flow rate to abdominal intestinal organs,
q_{SP}=0.75·Q_{L, init} (L·min⁻¹), and
q_{PS} is anatomic shunt flow rate, (L·min⁻¹) which bypasses the liver.

15. The method of claim 14, wherein the calculating total hepatic venous return flow rate to systemic circulation (q_{LS}) comprises: q_{LS}=q_{PL}+q_{SL}.

16. The method of claim 4, wherein estimating the hepatic extraction in the subject comprises calculating a fast phase extraction ratio (ER_{fast}), fast phase clearance of intravenously administered distinguishable cholate (Cl_{IV}), and slow phase of hepatic clearance (Cl_{H}) in the subject.

17. The method of claim 16, wherein the fast phase extraction ratio (ER_{fast}) is calculated as the ratio of total IV clearance (Cl_{IV}) (0 to 180 min) to first phase clearance (Cl_{FP}) (0-20 min)
$${ER}_{fast} = \frac{{Cl}_{IV}}{{Cl}_{FP}} = \frac{{C}_{0}}{{k}_{fast} \cdot {AUC}_{IV}} ,$$
wherein
AUC_{IV} is the area under the IV curve;
k_{fast} is estimated from the slope of log concentration-time IV curves between 5- and 20-minute timepoints,
$${k}_{fast} = - \frac{\text{?}}{\text{?}} ( {min}^{- 1} ) ;$$
$$\text{?} \text{indicates text missing or illegible when filed}$$
and
and C₀ is the extrapolated initial intravenously administered distinguishable cholate concentration
$${C}_{0} = - \frac{\text{?}}{\text{?}} ( μmol \cdot {L}^{- 1} ) ,$$
$$\text{?} \text{indicates text missing or illegible when filed}$$
wherein C(5) is the concentration of intravenously administered distinguishable cholate in the sample collected at 5 minutes after administration.

18. The method of claim 16, wherein the fast phase clearance of intravenously administered distinguishable cholate (Cl_{IV}) is calculated as
Cl_{IV}=Q_{L}·ER_{fast} (L·min⁻¹).

19. The method of claim 16, wherein the slow phase of hepatic clearance (Cl_{H}) in the subject is the hepatic clearance of orally administered distinguishable cholate and slow phase of intravenously administered distinguishable cholate clearance in the subject, optionally calculated as
Cl_{H}=Q_{L}·ER_{slow} (L·min⁻¹).

20. The method of claim 5, wherein the estimating cholate binding in the subject comprises modeling (1) albumin-bound noncellular intravascular distinguishable cholate, (2) extravascular distinguishable cholate distributed to cells/tissues, and (3) free/unbound distinguishable cholate.

21. The method of claim 20, wherein the modeling of albumin-bound noncellular intravascular distinguishable cholate comprises
estimating an initial albumin-bound noncellular intravascular fraction of total dose estimate (f_{A, init}) comprising calculating the ratio of systemic compartment volume (Vs) to the volume of distribution (Vd)
f_{A, init}=V_{S}/V_{d},
wherein V_{d}=D_{IV}/C₀ (L), D_{IV} is intravenous dose of distinguishable cholate, and C₀ and V_{S} are as defined above.

22. The method of claim 21, wherein the actual intravascular fraction (f_{A}) is determined comprising parameter estimation comprising weighted nonlinear least-squares regression at each distinguishable cholate concentration.

23. The method of claim 22, wherein the weights are reduced to reflect higher uncertainty during rapid distribution phase of IV clearance, and at low concentrations of oral clearance, optionally wherein the 5 min IV distinguishable cholate weight is 0.33, and the 5 min and 90 min oral distinguishable cholate weights are 0.67.

24. The method of claim 1, wherein the one or more indices of hepatic disease is selected from the group consisting of portal hepatic filtration rate (HFRp), systemic hepatic filtration rate (HFRs), cholate SHUNT, STAT, liver disease severity index (DSI), and hepatic reserve (HR) in the subject.

25. The method of claim 24, wherein the portal hepatic filtration rate (HFRp) is a clearance adjusted for body weight (BW) of the subject
$$H F {R}_{p} = \frac{{D}_{PO}}{{AUC}_{PO} \cdot B W} ,$$
wherein
D_{PO} is oral dose of the distinguishable cholate;
AUC_{PO} is area under the oral distinguishable cholate blood or serum sample concentration curve over the multiplicity of time points after administration.

26. The method of claim 24, wherein the systemic hepatic filtration rate (HFRs) is a clearance adjusted for body weight (BW) of the subject
$$H F {R}_{s} = \frac{{D}_{IV}}{{AUC}_{IV} \cdot B W} ,$$
wherein
D_{IV} is intravenous dose of the distinguishable cholate;
AUC_{IV} is area under the intravenous distinguishable cholate blood or serum sample concentration curve over the multiplicity of time points after administration.

27. The method of claim 24, wherein the cholate SHUNT (F) is a comparison of bioavailability of orally administered distinguishable cholate with bioavailability of IV-administered distinguishable calculated by the ratio of areas under the oral and IV distinguishable cholate concentration curves (AUC_{PO} and AUC_{IV}, respectively) and normalized by the IV dose (D_{IV}) and oral dose (D_{PO}), optionally wherein the SHUNT is calculated comprising
$$F = \frac{{AUC}_{PO} \cdot {D}_{IV}}{{AUC}_{IV} \cdot {D}_{PO}} .$$

28. The method of claim 24, wherein the STAT is a single point blood or serum oral distinguishable cholate estimate of liver disease severity index (DSI).

29. The method of claim 24, wherein the DSI is a score indicative of overall liver function comprising both portal and systemic HFR, optionally wherein
$$DSI = A \cdot \sqrt{{( ln [ \frac{H F {R}_{P , max}}{H F {R}_{P}} ] )}^{2} + {( ln [ \frac{H F {R}_{S , max}}{H F {R}_{S}} ] )}^{2}} ,$$
wherein HFR_{P,max} and HFR_{S,max} are the upper limits of clearance of healthy controls, and
A is a factor to scale DSI score from 0 to 50.

30. The method of claim 24, wherein the hepatic reserve (HR) is a numerical index representing overall hepatic health with HR values between 0 and 100
$$H R = 100 - A \cdot \sqrt{{( ln [ \frac{H F {R}_{P , lean}}{H F {R}_{P}} ] )}^{2} + {( ln [ \frac{H F {R}_{S , lean}}{H F {R}_{S}} ] )}^{2}} ,$$
wherein
HFR_{P} and HFR_{S} are indexed to lean controls minus one standard deviation (HFR_{P,lean} and
HFR_{S,lean}); and A is a constant integer from 100 to 0.

31. The method of claim 1, wherein the first distinguishable cholate compound is a first stable isotope labeled cholate and the second distinguishable cholate compound is a second stable isotope labeled cholate.

32. (canceled)

33. The method of claim 1, further comprising
receiving a plurality of blood or serum samples collected from the subject having or suspected of having or contracting a chronic liver disease, following oral administration of a dose of the first distinguishable cholate compound (DPO) to the subject and simultaneous intravenous co-administration of a dose of a second distinguishable cholate compound (DIV) to the subject, wherein the samples have been collected over intervals spanning a period of time after administration; and
quantifying the concentration of the first and the second distinguishable cholate compounds in each sample.

34. The method of claim 1, wherein the fitting comprises
generating an individualized oral clearance curve from the concentration of the first distinguishable cholate compound in each sample comprising using a computer algorithm curve fitting to the compartmental model and computing the area under the individualized oral clearance curve (AUC_{PO});
generating an individualized intravenous clearance curve from the concentration of the second distinguishable cholate compound in each sample by use of a computer algorithm curve fitting to the compartmental model and computing the area under the individualized intravenous clearance curve (AUC_{IV}).

35. The method of claim 1, wherein the samples have been collected from the subject over intervals comprising from one to seven time points after administration.

36.-37. (canceled)

38. The method of claim 1, wherein the liver disease is a chronic liver disease selected from the group consisting of chronic hepatitis C (CHC), chronic hepatitis B, alcoholic liver disease, Alcoholic SteatoHepatitis (ASH), and Non-Alcoholic Fatty Liver Disease (NAFLD), steatosis, Non-Alcoholic SteatoHepatitis (NASH), autoimmune liver disease, cryptogenic cirrhosis, hemochromatosis, Wilson's disease, alpha-1-antitrypsin deficiency, liver cancer, liver failure, cirrhosis, primary sclerosing cholangitis (PSC), and other cholestatic liver diseases.

39. The method of claim 3, wherein the clinical outcome is selected from the group consisting of Child-Turcotte-Pugh (CTP) progression, Model for End-stage Liver Disease (MELD) progression, variceal hemorrhage, ascites, splenomegaly, varices, portal hypertension (PHTN), hepatic encephalopathy, hepatocellular carcinoma (HCC), decompensation, or liver-related death.

40. The method of claim 3, wherein the treatment is selected from the group consisting of antiviral treatments, antifibrotic treatments, antibiotics, immunosuppressive treatments, anti-cancer treatments, ursodeoxycholic acid, farnesoid X receptor ligands, insulin sensitizing agents, interventional treatment, liver transplant, lifestyle changes, dietary restrictions, low glycemic index diet, antioxidants, vitamin supplements, transjugular intrahepatic portosystemic shunt (TIPS), catheter-directed thrombolysis, balloon dilation and stent placement, balloon-dilation and drainage, weight loss, exercise, and avoidance of alcohol.

41. The method of claim 3, wherein determining the need for treatment in the subject comprises
determining the one or more indices of hepatic disease in the subject; and
comparing the one or more indices of hepatic disease to one or more cutoff value(s),
wherein a change in the one or more indices of hepatic disease compared to cutoff value(s) is indicative of the need for treatment in the subject.

42. The method of claim 41, wherein the cutoff value(s) are derived from one or more normal healthy controls, a group of known patients, or within the subject over time.

43.-45. (canceled)

46. The method of claim 14, wherein the anatomic shunt flow rate (q_{PS}) differentiates healthy controls from subjects with large varices.

47. The method of claim 14, wherein the rate of portal venous inflow to the liver (q_{PL}) differentiates healthy controls, subjects with no varices, subjects with small varices, and subjects with large varices.
